SEQUENCE LISTING (64 sequences)

SEQ ID NO: 1 - DNA, 18 bp, synthetic construct
  misc_feature 1..18: note = target sequence
  source 1..18: mol_type = other DNA
cagcagcagcagcagcag

SEQ ID NO: 2 - protein, 35 aa, synthetic construct
  REGION 1..35: note = PPR motif
VTYNTLIDGLCKSGKIEEALKLFKEMEEKGITPSV

SEQ ID NO: 3 - protein, 35 aa, synthetic construct
  REGION 1..35: note = PPR motif
VTYNTLIDELCKSGKIEEALKLFKEMEEKGITPSV

SEQ ID NO: 4 - protein, 35 aa, synthetic construct
  REGION 1..35: note = PPR motif, 1st_U (v3.2_U)
VTYNTNIDQLCKSGKIEEALKLFKEMEEKGITPSV

SEQ ID NO: 5 - protein, 35 aa, synthetic construct
  REGION 1..35: note = PPR motif
VTYNTNIDELCKSGKIEEALKLFKEMEEKGITPSV

SEQ ID NO: 6 - protein, 35 aa, synthetic construct
  REGION 1..35: note = PPR motif
VTYNTNIDKLCKSGKIEEALKLFKEMEEKGITPSV

SEQ ID NO: 7 - protein, 35 aa, synthetic construct
  REGION 1..35: note = PPR motif
VTYNTDIDGLCKSGKIEEALKLFKEMEEKGITPSV

SEQ ID NO: 8 - protein, 35 aa, synthetic construct
  REGION 1..35: note = PPR motif
VTYTTLIDGLCKAGDVDEALELFKEMRSKGVKPNV

SEQ ID NO: 9 - protein, 35 aa, synthetic construct
  REGION 1..35: note = PPR motif
VTYTTLIDGLCKAGKVDEALELFDEMKERGIKPDV

SEQ ID NO: 10 - protein, 35 aa, synthetic construct
  REGION 1..35: note = PPR motif
VTYNTLIDGLCKAGRLDEAEELLEEMEEKGIKPDV

SEQ ID NO: 11 - DNA, 2303 bp, synthetic construct
  misc_difference 1..2303: note = PPR gene
  misc_difference 1894: note = a, c, g, t or u
  misc_difference 1935: note = a, c, g, t or u
  misc_difference 1963: note = a, c, g, t or u
  misc_difference 2004: note = a, c, g, t or u
  misc_difference 2032: note = a, c, g, t or u
  misc_difference 2073: note = a, c, g, t or u
  misc_difference 2101: note = a, c, g, t or u
  misc_difference 2142: note = a, c, g, t or u
  misc_difference 2170: note = a, c, g, t or u
  misc_difference 2211: note = a, c, g, t or u
  misc_difference 2239: note = a, c, g, t or u
  misc_difference 2280: note = a, c, g, t or u
  source 1..2303: mol_type = other DNA
gtcacatacaacaccctgatcgacggcctgtgcaagagcggcaagatcgaggaggccctgaagctgttcaaggagatggaggagaagggcatcacccccagcgtggtcacatacaccacactgatcgacggactgtgtaaagccggcgacgtggacgaagccctcgagctgttcaaagagatgcggagcaagggcgtgaagcccaacgtggtgacatacaccaccctgatcgacggcctgtgcaaggccggcaaagtggacgaggccctggagctgttcgacgagatgaaggagaggggcatcaagcccgacgtggtcacatacaacaccctgatcgacggcctgtgcaagagcggcaagatcgaggaggccctgaagctgttcaaggagatggaggagaagggcatcacccccagcgtggttacatacaccacactgatcgacggactgtgtaaagccggcgacgtggacgaagccctcgagctgttcaaagagatgcggagcaagggcgtgaagcccaacgtggtcacatacaccaccctgatcgacggcctgtgcaaggccggcaaggtggatgaggccctggagctgttcgacgagatgaaggagaggggcatcaagcccgacgtggttacatacaacaccctgatcgacggcctgtgcaagagcggcaagatcgaggaggccctgaagctgttcaaggagatggaggagaagggcatcacccccagcgtggtcacatacaccacactgatcgacggactgtgtaaagccggcgacgtggacgaagccctcgagctgttcaaagagatgcggagcaagggcgtgaagcccaacgtggtgacatacaccaccctgatcgacggcctgtgcaaggccggcaaagtggacgaggccctggagctgttcgacgagatgaaggagaggggcatcaagcccgacgtggtcacatacaacaccctgatcgacggcctgtgcaagagcggcaagatcgaggaggccctgaagctgttcaaggagatggaggagaagggcatcacccccagcgtggttacatacaccacactgatcgacggactgtgtaaagccggcgacgtggacgaagccctcgagctgttcaaagagatgcggagcaagggcgtgaagcccaacgtggtcacatacaccaccctgatcgacggcctgtgcaaggccggcaaggtggatgaggccctggagctgttcgacgagatgaaggagaggggcatcaagcccgacgtggtcacatacaacaccctgatcgacggcctgtgcaagagcggcaagatcgaggaggccctgaagctgttcaaggagatggaggagaagggcatcacccccagcgtggtcacatacaccacactgatcgacggactgtgtaaagccggcgacgtggacgaagccctcgagctgttcaaagagatgcggagcaagggcgtgaagcccaacgtggttacatacaccaccctgatcgacggcctgtgcaaggccggcaaagtggacgaggccctggagctgttcgacgagatgaaggagaggggcatcaagcccgacgtggtcacatacaacaccctgatcgacggcctgtgcaagagcggcaagatcgaggaggccctgaagctgttcaaggagatggaggagaagggcatcacccccagcgtggtgacatacaccacactgatcgacggactgtgtaaagccggcgacgtggacgaagccctcgagctgttcaaagagatgcggagcaagggcgtgaagcccaacgtggtcacatacaccaccctgatcgacggcctgtgcaaggccggcaaggtggatgaggccctggagctgttcgacgagatgaaggagaggggcatcaagcccgacgagvtyntdgcksgkakkmkgtsvvtyttdgckagdvdakmrskgvknvvtyttdgckagkvdadmkrgkdvvtyntdgcksgkakkmkgtsvvtyttdgckagdvdakmrskgvknvvtyttdgckagkvdadmkrgkdvvtyntdgcksgkakkmkgtsvvtyttdgckagdvdakmrskgvknvvtyttdgckagkvdadmkrgkdvvtyntdgcksgkakkmkgtsvvtyttdgckagdvdakmrskgvknvvtyttdgckagkvdadmkrgkdvvtyntdgcksgkakkmkgtsvvtyttdgckagdvdakmrskgvknvvtyttdgckagkvdadmkrgkdvvtyntdgcksgkakkmkgtsvvtyttdgckagdvdakmrskgvknvvtyttdgckagkvdadmkrgkd

SEQ ID NO: 12 - DNA, 2302 bp, synthetic construct
  misc_difference 1..2302: note = PPR gene
  misc_difference 1894: note = a, c, g, t or u
  misc_difference 1934: note = a, c, g, t or u
  misc_difference 1962: note = a, c, g, t or u
  misc_difference 2003: note = a, c, g, t or u
  misc_difference 2031: note = a, c, g, t or u
  misc_difference 2072: note = a, c, g, t or u
  misc_difference 2100: note = a, c, g, t or u
  misc_difference 2141: note = a, c, g, t or u
  misc_difference 2169: note = a, c, g, t or u
  misc_difference 2210: note = a, c, g, t or u
  misc_difference 2238: note = a, c, g, t or u
  misc_difference 2279: note = a, c, g, t or u
  source 1..2302: mol_type = other DNA
gtcacatacaacaccctgatcgacgaactgtgcaagagcggcaagatcgaggaggccctgaagctgttcaaggagatggaggagaagggcatcacccccagcgtggtcacatacaccacactgatcgacggactgtgtaaagccggcgacgtggacgaagccctcgagctgttcaaagagatgcggagcaagggcgtgaagcccaacgtggtgacatacaccaccctgatcgacggcctgtgcaaggccggcaaagtggacgaggccctggagctgttcgacgagatgaaggagaggggcatcaagcccgacgtggtcacatacaacaccctgatcgacggcctgtgcaagagcggcaagatcgaggaggccctgaagctgttcaaggagatggaggagaagggcatcacccccagcgtggttacatacaccacactgatcgacggactgtgtaaagccggcgacgtggacgaagccctcgagctgttcaaagagatgcggagcaagggcgtgaagcccaacgtggtcacatacaccaccctgatcgacggcctgtgcaaggccggcaaggtggatgaggccctggagctgttcgacgagatgaaggagaggggcatcaagcccgacgtggttacatacaacaccctgatcgacggcctgtgcaagagcggcaagatcgaggaggccctgaagctgttcaaggagatggaggagaagggcatcacccccagcgtggtcacatacaccacactgatcgacggactgtgtaaagccggcgacgtggacgaagccctcgagctgttcaaagagatgcggagcaagggcgtgaagcccaacgtggtgacatacaccaccctgatcgacggcctgtgcaaggccggcaaagtggacgaggccctggagctgttcgacgagatgaaggagaggggcatcaagcccgacgtggtcacatacaacaccctgatcgacggcctgtgcaagagcggcaagatcgaggaggccctgaagctgttcaaggagatggaggagaagggcatcacccccagcgtggttacatacaccacactgatcgacggactgtgtaaagccggcgacgtggacgaagccctcgagctgttcaaagagatgcggagcaagggcgtgaagcccaacgtggtcacatacaccaccctgatcgacggcctgtgcaaggccggcaaggtggatgaggccctggagctgttcgacgagatgaaggagaggggcatcaagcccgacgtggtcacatacaacaccctgatcgacggcctgtgcaagagcggcaagatcgaggaggccctgaagctgttcaaggagatggaggagaagggcatcacccccagcgtggtcacatacaccacactgatcgacggactgtgtaaagccggcgacgtggacgaagccctcgagctgttcaaagagatgcggagcaagggcgtgaagcccaacgtggttacatacaccaccctgatcgacggcctgtgcaaggccggcaaagtggacgaggccctggagctgttcgacgagatgaaggagaggggcatcaagcccgacgtggtcacatacaacaccctgatcgacggcctgtgcaagagcggcaagatcgaggaggccctgaagctgttcaaggagatggaggagaagggcatcacccccagcgtggtgacatacaccacactgatcgacggactgtgtaaagccggcgacgtggacgaagccctcgagctgttcaaagagatgcggagcaagggcgtgaagcccaacgtggtcacatacaccaccctgatcgacggcctgtgcaaggccggcaaggtggatgaggccctggagctgttcgacgagatgaaggagaggggcatcaagcccgacgagvtyntdcksgkakkmkgtsvvtyttdgckagdvdakmrskgvknvvtyttdgckagkvdadmkrgkdvvtyntdgcksgkakkmkgtsvvtyttdgckagdvdakmrskgvknvvtyttdgckagkvdadmkrgkdvvtyntdgcksgkakkmkgtsvvtyttdgckagdvdakmrskgvknvvtyttdgckagkvdadmkrgkdvvtyntdgcksgkakkmkgtsvvtyttdgckagdvdakmrskgvknvvtyttdgckagkvdadmkrgkdvvtyntdgcksgkakkmkgtsvvtyttdgckagdvdakmrskgvknvvtyttdgckagkvdadmkrgkdvvtyntdgcksgkakkmkgtsvvtyttdgckagdvdakmrskgvknvvtyttdgckagkvdadmkrgkd

SEQ ID NO: 13 - DNA, 2303 bp, synthetic construct
  misc_difference 1..2303: note = PPR gene
  misc_difference 1894: note = a, c, g, t or u
  misc_difference 1896: note = a, c, g, t or u
  misc_difference 1935: note = a, c, g, t or u
  misc_difference 1963: note = a, c, g, t or u
  misc_difference 2004: note = a, c, g, t or u
  misc_difference 2032: note = a, c, g, t or u
  misc_difference 2073: note = a, c, g, t or u
  misc_difference 2101: note = a, c, g, t or u
  misc_difference 2142: note = a, c, g, t or u
  misc_difference 2170: note = a, c, g, t or u
  misc_difference 2211: note = a, c, g, t or u
  misc_difference 2239: note = a, c, g, t or u
  misc_difference 2280: note = a, c, g, t or u
  source 1..2303: mol_type = other DNA
gtcacatacaacaccaacatcgaccagctgtgcaagagcggcaagatcgaggaggccctgaagctgttcaaggagatggaggagaagggcatcacccccagcgtggtcacatacaccacactgatcgacggactgtgtaaagccggcgacgtggacgaagccctcgagctgttcaaagagatgcggagcaagggcgtgaagcccaacgtggtgacatacaccaccctgatcgacggcctgtgcaaggccggcaaagtggacgaggccctggagctgttcgacgagatgaaggagaggggcatcaagcccgacgtggtcacatacaacaccctgatcgacggcctgtgcaagagcggcaagatcgaggaggccctgaagctgttcaaggagatggaggagaagggcatcacccccagcgtggttacatacaccacactgatcgacggactgtgtaaagccggcgacgtggacgaagccctcgagctgttcaaagagatgcggagcaagggcgtgaagcccaacgtggtcacatacaccaccctgatcgacggcctgtgcaaggccggcaaggtggatgaggccctggagctgttcgacgagatgaaggagaggggcatcaagcccgacgtggttacatacaacaccctgatcgacggcctgtgcaagagcggcaagatcgaggaggccctgaagctgttcaaggagatggaggagaagggcatcacccccagcgtggtcacatacaccacactgatcgacggactgtgtaaagccggcgacgtggacgaagccctcgagctgttcaaagagatgcggagcaagggcgtgaagcccaacgtggtgacatacaccaccctgatcgacggcctgtgcaaggccggcaaagtggacgaggccctggagctgttcgacgagatgaaggagaggggcatcaagcccgacgtggtcacatacaacaccctgatcgacggcctgtgcaagagcggcaagatcgaggaggccctgaagctgttcaaggagatggaggagaagggcatcacccccagcgtggttacatacaccacactgatcgacggactgtgtaaagccggcgacgtggacgaagccctcgagctgttcaaagagatgcggagcaagggcgtgaagcccaacgtggtcacatacaccaccctgatcgacggcctgtgcaaggccggcaaggtggatgaggccctggagctgttcgacgagatgaaggagaggggcatcaagcccgacgtggtcacatacaacaccctgatcgacggcctgtgcaagagcggcaagatcgaggaggccctgaagctgttcaaggagatggaggagaagggcatcacccccagcgtggtcacatacaccacactgatcgacggactgtgtaaagccggcgacgtggacgaagccctcgagctgttcaaagagatgcggagcaagggcgtgaagcccaacgtggttacatacaccaccctgatcgacggcctgtgcaaggccggcaaagtggacgaggccctggagctgttcgacgagatgaaggagaggggcatcaagcccgacgtggtcacatacaacaccctgatcgacggcctgtgcaagagcggcaagatcgaggaggccctgaagctgttcaaggagatggaggagaagggcatcacccccagcgtggtgacatacaccacactgatcgacggactgtgtaaagccggcgacgtggacgaagccctcgagctgttcaaagagatgcggagcaagggcgtgaagcccaacgtggtcacatacaccaccctgatcgacggcctgtgcaaggccggcaaggtggatgaggccctggagctgttcgacgagatgaaggagaggggcatcaagcccgacgagvtyntndcksgkakkmkgtsvvtyttdgckagdvdakmrskgvknvvtyttdgckagkvdadmkrgkdvvtyntdgcksgkakkmkgtsvvtyttdgckagdvdakmrskgvknvvtyttdgckagkvdadmkrgkdvvtyntdgcksgkakkmkgtsvvtyttdgckagdvdakmrskgvknvvtyttdgckagkvdadmkrgkdvvtyntdgcksgkakkmkgtsvvtyttdgckagdvdakmrskgvknvvtyttdgckagkvdadmkrgkdvvtyntdgcksgkakkmkgtsvvtyttdgckagdvdakmrskgvknvvtyttdgckagkvdadmkrgkdvvtyntdgcksgkakkmkgtsvvtyttdgckagdvdakmrskgvknvvtyttdgckagkvdadmkrgkd

SEQ ID NO: 14 - DNA, 2303 bp, synthetic construct
  misc_difference 1..2303: note = PPR gene
  misc_difference 1894: note = a, c, g, t or u
  misc_difference 1896: note = a, c, g, t or u
  misc_difference 1935: note = a, c, g, t or u
  misc_difference 1963: note = a, c, g, t or u
  misc_difference 2004: note = a, c, g, t or u
  misc_difference 2032: note = a, c, g, t or u
  misc_difference 2073: note = a, c, g, t or u
  misc_difference 2101: note = a, c, g, t or u
  misc_difference 2142: note = a, c, g, t or u
  misc_difference 2170: note = a, c, g, t or u
  misc_difference 2211: note = a, c, g, t or u
  misc_difference 2239: note = a, c, g, t or u
  misc_difference 2280: note = a, c, g, t or u
  source 1..2303: mol_type = other DNA
gtcacatacaacaccaacatcgacgaactgtgcaagagcggcaagatcgaggaggccctgaagctgttcaaggagatggaggagaagggcatcacccccagcgtggtcacatacaccacactgatcgacggactgtgtaaagccggcgacgtggacgaagccctcgagctgttcaaagagatgcggagcaagggcgtgaagcccaacgtggtgacatacaccaccctgatcgacggcctgtgcaaggccggcaaagtggacgaggccctggagctgttcgacgagatgaaggagaggggcatcaagcccgacgtggtcacatacaacaccctgatcgacggcctgtgcaagagcggcaagatcgaggaggccctgaagctgttcaaggagatggaggagaagggcatcacccccagcgtggttacatacaccacactgatcgacggactgtgtaaagccggcgacgtggacgaagccctcgagctgttcaaagagatgcggagcaagggcgtgaagcccaacgtggtcacatacaccaccctgatcgacggcctgtgcaaggccggcaaggtggatgaggccctggagctgttcgacgagatgaaggagaggggcatcaagcccgacgtggttacatacaacaccctgatcgacggcctgtgcaagagcggcaagatcgaggaggccctgaagctgttcaaggagatggaggagaagggcatcacccccagcgtggtcacatacaccacactgatcgacggactgtgtaaagccggcgacgtggacgaagccctcgagctgttcaaagagatgcggagcaagggcgtgaagcccaacgtggtgacatacaccaccctgatcgacggcctgtgcaaggccggcaaagtggacgaggccctggagctgttcgacgagatgaaggagaggggcatcaagcccgacgtggtcacatacaacaccctgatcgacggcctgtgcaagagcggcaagatcgaggaggccctgaagctgttcaaggagatggaggagaagggcatcacccccagcgtggttacatacaccacactgatcgacggactgtgtaaagccggcgacgtggacgaagccctcgagctgttcaaagagatgcggagcaagggcgtgaagcccaacgtggtcacatacaccaccctgatcgacggcctgtgcaaggccggcaaggtggatgaggccctggagctgttcgacgagatgaaggagaggggcatcaagcccgacgtggtcacatacaacaccctgatcgacggcctgtgcaagagcggcaagatcgaggaggccctgaagctgttcaaggagatggaggagaagggcatcacccccagcgtggtcacatacaccacactgatcgacggactgtgtaaagccggcgacgtggacgaagccctcgagctgttcaaagagatgcggagcaagggcgtgaagcccaacgtggttacatacaccaccctgatcgacggcctgtgcaaggccggcaaagtggacgaggccctggagctgttcgacgagatgaaggagaggggcatcaagcccgacgtggtcacatacaacaccctgatcgacggcctgtgcaagagcggcaagatcgaggaggccctgaagctgttcaaggagatggaggagaagggcatcacccccagcgtggtgacatacaccacactgatcgacggactgtgtaaagccggcgacgtggacgaagccctcgagctgttcaaagagatgcggagcaagggcgtgaagcccaacgtggtcacatacaccaccctgatcgacggcctgtgcaaggccggcaaggtggatgaggccctggagctgttcgacgagatgaaggagaggggcatcaagcccgacgagvtyntndcksgkakkmkgtsvvtyttdgckagdvdakmrskgvknvvtyttdgckagkvdadmkrgkdvvtyntdgcksgkakkmkgtsvvtyttdgckagdvdakmrskgvknvvtyttdgckagkvdadmkrgkdvvtyntdgcksgkakkmkgtsvvtyttdgckagdvdakmrskgvknvvtyttdgckagkvdadmkrgkdvvtyntdgcksgkakkmkgtsvvtyttdgckagdvdakmrskgvknvvtyttdgckagkvdadmkrgkdvvtyntdgcksgkakkmkgtsvvtyttdgckagdvdakmrskgvknvvtyttdgckagkvdadmkrgkdvvtyntdgcksgkakkmkgtsvvtyttdgckagdvdakmrskgvknvvtyttdgckagkvdadmkrgkd

SEQ ID NO: 15 - DNA, 2304 bp, synthetic construct
  misc_difference 1..2304: note = PPR gene
  misc_difference 1894: note = a, c, g, t or u
  misc_difference 1896: note = a, c, g, t or u
  misc_difference 1936: note = a, c, g, t or u
  misc_difference 1964: note = a, c, g, t or u
  misc_difference 2005: note = a, c, g, t or u
  misc_difference 2033: note = a, c, g, t or u
  misc_difference 2074: note = a, c, g, t or u
  misc_difference 2102: note = a, c, g, t or u
  misc_difference 2143: note = a, c, g, t or u
  misc_difference 2171: note = a, c, g, t or u
  misc_difference 2212: note = a, c, g, t or u
  misc_difference 2240: note = a, c, g, t or u
  misc_difference 2281: note = a, c, g, t or u
  source 1..2304: mol_type = other DNA
gtcacatacaacaccaacatcgacaaactgtgcaagagcggcaagatcgaggaggccctgaagctgttcaaggagatggaggagaagggcatcacccccagcgtggtcacatacaccacactgatcgacggactgtgtaaagccggcgacgtggacgaagccctcgagctgttcaaagagatgcggagcaagggcgtgaagcccaacgtggtgacatacaccaccctgatcgacggcctgtgcaaggccggcaaagtggacgaggccctggagctgttcgacgagatgaaggagaggggcatcaagcccgacgtggtcacatacaacaccctgatcgacggcctgtgcaagagcggcaagatcgaggaggccctgaagctgttcaaggagatggaggagaagggcatcacccccagcgtggttacatacaccacactgatcgacggactgtgtaaagccggcgacgtggacgaagccctcgagctgttcaaagagatgcggagcaagggcgtgaagcccaacgtggtcacatacaccaccctgatcgacggcctgtgcaaggccggcaaggtggatgaggccctggagctgttcgacgagatgaaggagaggggcatcaagcccgacgtggttacatacaacaccctgatcgacggcctgtgcaagagcggcaagatcgaggaggccctgaagctgttcaaggagatggaggagaagggcatcacccccagcgtggtcacatacaccacactgatcgacggactgtgtaaagccggcgacgtggacgaagccctcgagctgttcaaagagatgcggagcaagggcgtgaagcccaacgtggtgacatacaccaccctgatcgacggcctgtgcaaggccggcaaagtggacgaggccctggagctgttcgacgagatgaaggagaggggcatcaagcccgacgtggtcacatacaacaccctgatcgacggcctgtgcaagagcggcaagatcgaggaggccctgaagctgttcaaggagatggaggagaagggcatcacccccagcgtggttacatacaccacactgatcgacggactgtgtaaagccggcgacgtggacgaagccctcgagctgttcaaagagatgcggagcaagggcgtgaagcccaacgtggtcacatacaccaccctgatcgacggcctgtgcaaggccggcaaggtggatgaggccctggagctgttcgacgagatgaaggagaggggcatcaagcccgacgtggtcacatacaacaccctgatcgacggcctgtgcaagagcggcaagatcgaggaggccctgaagctgttcaaggagatggaggagaagggcatcacccccagcgtggtcacatacaccacactgatcgacggactgtgtaaagccggcgacgtggacgaagccctcgagctgttcaaagagatgcggagcaagggcgtgaagcccaacgtggttacatacaccaccctgatcgacggcctgtgcaaggccggcaaagtggacgaggccctggagctgttcgacgagatgaaggagaggggcatcaagcccgacgtggtcacatacaacaccctgatcgacggcctgtgcaagagcggcaagatcgaggaggccctgaagctgttcaaggagatggaggagaagggcatcacccccagcgtggtgacatacaccacactgatcgacggactgtgtaaagccggcgacgtggacgaagccctcgagctgttcaaagagatgcggagcaagggcgtgaagcccaacgtggtcacatacaccaccctgatcgacggcctgtgcaaggccggcaaggtggatgaggccctggagctgttcgacgagatgaaggagaggggcatcaagcccgacgagvtyntndkcksgkakkmkgtsvvtyttdgckagdvdakmrskgvknvvtyttdgckagkvdadmkrgkdvvtyntdgcksgkakkmkgtsvvtyttdgckagdvdakmrskgvknvvtyttdgckagkvdadmkrgkdvvtyntdgcksgkakkmkgtsvvtyttdgckagdvdakmrskgvknvvtyttdgckagkvdadmkrgkdvvtyntdgcksgkakkmkgtsvvtyttdgckagdvdakmrskgvknvvtyttdgckagkvdadmkrgkdvvtyntdgcksgkakkmkgtsvvtyttdgckagdvdakmrskgvknvvtyttdgckagkvdadmkrgkdvvtyntdgcksgkakkmkgtsvvtyttdgckagdvdakmrskgvknvvtyttdgckagkvdadmkrgkd

SEQ ID NO: 16 - DNA, 2304 bp, synthetic construct
  misc_difference 1..2304: note = PPR gene
  misc_difference 1894: note = a, c, g, t or u
  misc_difference 1936: note = a, c, g, t or u
  misc_difference 1964: note = a, c, g, t or u
  misc_difference 2005: note = a, c, g, t or u
  misc_difference 2033: note = a, c, g, t or u
  misc_difference 2074: note = a, c, g, t or u
  misc_difference 2102: note = a, c, g, t or u
  misc_difference 2143: note = a, c, g, t or u
  misc_difference 2171: note = a, c, g, t or u
  misc_difference 2212: note = a, c, g, t or u
  misc_difference 2240: note = a, c, g, t or u
  misc_difference 2281: note = a, c, g, t or u
  source 1..2304: mol_type = other DNA
gtcacatacaacaccgatatcgacggcctgtgcaagagcggcaagatcgaggaggccctgaagctgttcaaggagatggaggagaagggcatcacccccagcgtggtcacatacaccacactgatcgacggactgtgtaaagccggcgacgtggacgaagccctcgagctgttcaaagagatgcggagcaagggcgtgaagcccaacgtggtgacatacaccaccctgatcgacggcctgtgcaaggccggcaaagtggacgaggccctggagctgttcgacgagatgaaggagaggggcatcaagcccgacgtggtcacatacaacaccctgatcgacggcctgtgcaagagcggcaagatcgaggaggccctgaagctgttcaaggagatggaggagaagggcatcacccccagcgtggttacatacaccacactgatcgacggactgtgtaaagccggcgacgtggacgaagccctcgagctgttcaaagagatgcggagcaagggcgtgaagcccaacgtggtcacatacaccaccctgatcgacggcctgtgcaaggccggcaaggtggatgaggccctggagctgttcgacgagatgaaggagaggggcatcaagcccgacgtggttacatacaacaccctgatcgacggcctgtgcaagagcggcaagatcgaggaggccctgaagctgttcaaggagatggaggagaagggcatcacccccagcgtggtcacatacaccacactgatcgacggactgtgtaaagccggcgacgtggacgaagccctcgagctgttcaaagagatgcggagcaagggcgtgaagcccaacgtggtgacatacaccaccctgatcgacggcctgtgcaaggccggcaaagtggacgaggccctggagctgttcgacgagatgaaggagaggggcatcaagcccgacgtggtcacatacaacaccctgatcgacggcctgtgcaagagcggcaagatcgaggaggccctgaagctgttcaaggagatggaggagaagggcatcacccccagcgtggttacatacaccacactgatcgacggactgtgtaaagccggcgacgtggacgaagccctcgagctgttcaaagagatgcggagcaagggcgtgaagcccaacgtggtcacatacaccaccctgatcgacggcctgtgcaaggccggcaaggtggatgaggccctggagctgttcgacgagatgaaggagaggggcatcaagcccgacgtggtcacatacaacaccctgatcgacggcctgtgcaagagcggcaagatcgaggaggccctgaagctgttcaaggagatggaggagaagggcatcacccccagcgtggtcacatacaccacactgatcgacggactgtgtaaagccggcgacgtggacgaagccctcgagctgttcaaagagatgcggagcaagggcgtgaagcccaacgtggttacatacaccaccctgatcgacggcctgtgcaaggccggcaaagtggacgaggccctggagctgttcgacgagatgaaggagaggggcatcaagcccgacgtggtcacatacaacaccctgatcgacggcctgtgcaagagcggcaagatcgaggaggccctgaagctgttcaaggagatggaggagaagggcatcacccccagcgtggtgacatacaccacactgatcgacggactgtgtaaagccggcgacgtggacgaagccctcgagctgttcaaagagatgcggagcaagggcgtgaagcccaacgtggtcacatacaccaccctgatcgacggcctgtgcaaggccggcaaggtggatgaggccctggagctgttcgacgagatgaaggagaggggcatcaagcccgacgagvtyntddgcksgkakkmkgtsvvtyttdgckagdvdakmrskgvknvvtyttdgckagkvdadmkrgkdvvtyntdgcksgkakkmkgtsvvtyttdgckagdvdakmrskgvknvvtyttdgckagkvdadmkrgkdvvtyntdgcksgkakkmkgtsvvtyttdgckagdvdakmrskgvknvvtyttdgckagkvdadmkrgkdvvtyntdgcksgkakkmkgtsvvtyttdgckagdvdakmrskgvknvvtyttdgckagkvdadmkrgkdvvtyntdgcksgkakkmkgtsvvtyttdgckagdvdakmrskgvknvvtyttdgckagkvdadmkrgkdvvtyntdgcksgkakkmkgtsvvtyttdgckagdvdakmrskgvknvvtyttdgckagkvdadmkrgkd

SEQ ID NO: 17 - DNA, 3481 bp, synthetic construct
  misc_difference 1..3481: note = PPR vector
  misc_difference 2858: note = a, c, g, t or u
  misc_difference 2914: note = a, c, g, t or u
  misc_difference 2926: note = a, c, g, t or u
  misc_difference 2934: note = a, c, g, t or u
  misc_difference 2939: note = a, c, g, t or u
  misc_difference 2941: note = a, c, g, t or u
  misc_difference 2944: note = a, c, g, t or u
  misc_difference 2952: note = a, c, g, t or u
  misc_difference 2956: note = a, c, g, t or u
  misc_difference 2960: note = a, c, g, t or u
  misc_difference 2969: note = a, c, g, t or u
  misc_difference 2976: note = a, c, g, t or u
  misc_difference 2986: note = a, c, g, t or u
  misc_difference 3023: note = a, c, g, t or u
  misc_difference 3029: note = a, c, g, t or u
  misc_difference 3070: note = a, c, g, t or u
  misc_difference 3098: note = a, c, g, t or u
  misc_difference 3139: note = a, c, g, t or u
  misc_difference 3167: note = a, c, g, t or u
  misc_difference 3208: note = a, c, g, t or u
  misc_difference 3236: note = a, c, g, t or u
  misc_difference 3277: note = a, c, g, t or u
  misc_difference 3305: note = a, c, g, t or u
  misc_difference 3346: note = a, c, g, t or u
  misc_difference 3374: note = a, c, g, t or u
  misc_difference 3415: note = a, c, g, t or u
  misc_difference 3441: note = a, c, g, t or u
  source 1..3481: mol_type = other DNA
atggccggagtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagccaaagaaaaagagaaaggttagccatggctccggcggcagcgggggagggctccatatgggaaactccgtggtcacatacaacaccctgatcgacggcctgtgcaagagcggcaagatcgaggaggccctgaagctgttcaaggagatggaggagaagggcatcacccccagcgtggtcacatacaccacactgatcgacggactgtgtaaagccggcgacgtggacgaagccctcgagctgttcaaagagatgcggagcaagggcgtgaagcccaacgtggtgacatacaccaccctgatcgacggcctgtgcaaggccggcaaagtggacgaggccctggagctgttcgacgagatgaaggagaggggcatcaagcccgacgtggtcacatacaacaccctgatcgacggcctgtgcaagagcggcaagatcgaggaggccctgaagctgttcaaggagatggaggagaagggcatcacccccagcgtggttacatacaccacactgatcgacggactgtgtaaagccggcgacgtggacgaagccctcgagctgttcaaagagatgcggagcaagggcgtgaagcccaacgtggtcacatacaccaccctgatcgacggcctgtgcaaggccggcaaggtggatgaggccctggagctgttcgacgagatgaaggagaggggcatcaagcccgacgtggttacatacaacaccctgatcgacggcctgtgcaagagcggcaagatcgaggaggccctgaagctgttcaaggagatggaggagaagggcatcacccccagcgtggtcacatacaccacactgatcgacggactgtgtaaagccggcgacgtggacgaagccctcgagctgttcaaagagatgcggagcaagggcgtgaagcccaacgtggtgacatacaccaccctgatcgacggcctgtgcaaggccggcaaagtggacgaggccctggagctgttcgacgagatgaaggagaggggcatcaagcccgacgtggtcacatacaacaccctgatcgacggcctgtgcaagagcggcaagatcgaggaggccctgaagctgttcaaggagatggaggagaagggcatcacccccagcgtggttacatacaccacactgatcgacggactgtgtaaagccggcgacgtggacgaagccctcgagctgttcaaagagatgcggagcaagggcgtgaagcccaacgtggtcacatacaccaccctgatcgacggcctgtgcaaggccggcaaggtggatgaggccctggagctgttcgacgagatgaaggagaggggcatcaagcccgacgtggtcacatacaacaccctgatcgacggcctgtgcaagagcggcaagatcgaggaggccctgaagctgttcaaggagatggaggagaagggcatcacccccagcgtggtcacatacaccacactgatcgacggactgtgtaaagccggcgacgtggacgaagccctcgagctgttcaaagagatgcggagcaagggcgtgaagcccaacgtggttacatacaccaccctgatcgacggcctgtgcaaggccggcaaagtggacgaggccctggagctgttcgacgagatgaaggagaggggcatcaagcccgacgtggtcacatacaacaccctgatcgacggcctgtgcaagagcggcaagatcgaggaggccctgaagctgttcaaggagatggaggagaagggcatcacccccagcgtggtgacatacaccacactgatcgacggactgtgtaaagccggcgacgtggacgaagccctcgagctgttcaaagagatgcggagcaagggcgtgaagcccaacgtggtcacatacaccaccctgatcgacggcctgtgcaaggccggcaaggtggatgaggccctggagctgttcgacgagatgaaggagaggggcatcaagcccgacgagctgacctacaacaccctgatcagcggcctgggcaaggccggcagagccagagacccccccgtgctcagtagcggggactataaggaccacgacggagactacaaggatcatgatattgattacaaagacgatgacgataagatggccggacgctagmagvskgtgvvvdgdvnghksvsgggdatygktkcttgkvwtvtttygvcsrydhmkhdksamgyvrtkddgnyktravkgdtvnrkgdkdgnghkynynshnvymadkkngkvnkrhndgsvadhyntgdgvdnhystsaskdnkrdhmvvtaagtgmdykkkkrkvshgsggsggghmgnsvvtyntdgcksgkakkmkgtsvvtyttdgckagdvdakmrskgvknvvtyttdgckagkvdadmkrgkdvvtyntdgcksgkakkmkgtsvvtyttdgckagdvdakmrskgvknvvtyttdgckagkvdadmkrgkdvvtyntdgcksgkakkmkgtsvvtyttdgckagdvdakmrskgvknvvtyttdgckagkvdadmkrgkdvvtyntdgcksgkakkmkgtsvvtyttdgckagdvdakmrskgvknvvtyttdgckagkvdadmkrgkdvvtyntdgcksgkakkmkgtsvvtyttdgckagdvdakmrskgvknvvtyttdgckagkvdadmkrgkdvvtyntdgcksgkakkmkgtsvvtyttdgckagdvdakmrskgvknvvtyttdgckagkvdadmkrgkdtyntsggkagrardvssgdykdhdgdykdhddykddddkmagr

SEQ ID NO: 18 - DNA, 3480 bp, synthetic construct
  misc_difference 1..3480: note = PPR vector
  misc_difference 2858: note = a, c, g, t or u
  misc_difference 2914: note = a, c, g, t or u
  misc_difference 2926: note = a, c, g, t or u
  misc_difference 2934: note = a, c, g, t or u
  misc_difference 2939: note = a, c, g, t or u
  misc_difference 2941: note = a, c, g, t or u
  misc_difference 2944: note = a, c, g, t or u
  misc_difference 2952: note = a, c, g, t or u
  misc_difference 2956: note = a, c, g, t or u
  misc_difference 2960: note = a, c, g, t or u
  misc_difference 2969: note = a, c, g, t or u
  misc_difference 2976: note = a, c, g, t or u
  misc_difference 2986: note = a, c, g, t or u
  misc_difference 3023: note = a, c, g, t or u
  misc_difference 3029: note = a, c, g, t or u
  misc_difference 3069: note = a, c, g, t or u
  misc_difference 3097: note = a, c, g, t or u
  misc_difference 3138: note = a, c, g, t or u
  misc_difference 3166: note = a, c, g, t or u
  misc_difference 3207: note = a, c, g, t or u
  misc_difference 3235: note = a, c, g, t or u
  misc_difference 3276: note = a, c, g, t or u
  misc_difference 3304: note = a, c, g, t or u
  misc_difference 3345: note = a, c, g, t or u
  misc_difference 3373: note = a, c, g, t or u
  misc_difference 3414: note = a, c, g, t or u
  misc_difference 3440: note = a, c, g, t or u
  source 1..3480: mol_type = other DNA
atggccggagtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagccaaagaaaaagagaaaggttagccatggctccggcggcagcgggggagggctccatatgggaaactccgtggtcacatacaacaccctgatcgacgaactgtgcaagagcggcaagatcgaggaggccctgaagctgttcaaggagatggaggagaagggcatcacccccagcgtggtcacatacaccacactgatcgacggactgtgtaaagccggcgacgtggacgaagccctcgagctgttcaaagagatgcggagcaagggcgtgaagcccaacgtggtgacatacaccaccctgatcgacggcctgtgcaaggccggcaaagtggacgaggccctggagctgttcgacgagatgaaggagaggggcatcaagcccgacgtggtcacatacaacaccctgatcgacggcctgtgcaagagcggcaagatcgaggaggccctgaagctgttcaaggagatggaggagaagggcatcacccccagcgtggttacatacaccacactgatcgacggactgtgtaaagccggcgacgtggacgaagccctcgagctgttcaaagagatgcggagcaagggcgtgaagcccaacgtggtcacatacaccaccctgatcgacggcctgtgcaaggccggcaaggtggatgaggccctggagctgttcgacgagatgaaggagaggggcatcaagcccgacgtggttacatacaacaccctgatcgacggcctgtgcaagagcggcaagatcgaggaggccctgaagctgttcaaggagatggaggagaagggcatcacccccagcgtggtcacatacaccacactgatcgacggactgtgtaaagccggcgacgtggacgaagccctcgagctgttcaaagagatgcggagcaagggcgtgaagcccaacgtggtgacatacaccaccctgatcgacggcctgtgcaaggccggcaaagtggacgaggccctggagctgttcgacgagatgaaggagaggggcatcaagcccgacgtggtcacatacaacaccctgatcgacggcctgtgcaagagcggcaagatcgaggaggccctgaagctgttcaaggagatggaggagaagggcatcacccccagcgtggttacatacaccacactgatcgacggactgtgtaaagccggcgacgtggacgaagccctcgagctgttcaaagagatgcggagcaagggcgtgaagcccaacgtggtcacatacaccaccctgatcgacggcctgtgcaaggccggcaaggtggatgaggccctggagctgttcgacgagatgaaggagaggggcatcaagcccgacgtggtcacatacaacaccctgatcgacggcctgtgcaagagcggcaagatcgaggaggccctgaagctgttcaaggagatggaggagaagggcatcacccccagcgtggtcacatacaccacactgatcgacggactgtgtaaagccggcgacgtggacgaagccctcgagctgttcaaagagatgcggagcaagggcgtgaagcccaacgtggttacatacaccaccctgatcgacggcctgtgcaaggccggcaaagtggacgaggccctggagctgttcgacgagatgaaggagaggggcatcaagcccgacgtggtcacatacaacaccctgatcgacggcctgtgcaagagcggcaagatcgaggaggccctgaagctgttcaaggagatggaggagaagggcatcacccccagcgtggtgacatacaccacactgatcgacggactgtgtaaagccggcgacgtggacgaagccctcgagctgttcaaagagatgcggagcaagggcgtgaagcccaacgtggtcacatacaccaccctgatcgacggcctgtgcaaggccggcaaggtggatgaggccctggagctgttcgacgagatgaaggagaggggcatcaagcccgacgagctgacctacaacaccctgatcagcggcctgggcaaggccggcagagccagagacccccccgtgctcagtagcggggactataaggaccacgacggagactacaaggatcatgatattgattacaaagacgatgacgataagatggccggacgctagmagvskgtgvvvdgdvnghksvsgggdatygktkcttgkvwtvtttygvcsrydhmkhdksamgyvrtkddgnyktravkgdtvnrkgdkdgnghkynynshnvymadkkngkvnkrhndgsvadhyntgdgvdnhystsaskdnkrdhmvvtaagtgmdykkkkrkvshgsggsggghmgnsvvtyntdcksgkakkmkgtsvvtyttdgckagdvdakmrskgvknvvtyttdgckagkvdadmkrgkdvvtyntdgcksgkakkmkgtsvvtyttdgckagdvdakmrskgvknvvtyttdgckagkvdadmkrgkdvvtyntdgcksgkakkmkgtsvvtyttdgckagdvdakmrskgvknvvtyttdgckagkvdadmkrgkdvvtyntdgcksgkakkmkgtsvvtyttdgckagdvdakmrskgvknvvtyttdgckagkvdadmkrgkdvvtyntdgcksgkakkmkgtsvvtyttdgckagdvdakmrskgvknvvtyttdgckagkvdadmkrgkdvvtyntdgcksgkakkmkgtsvvtyttdgckagdvdakmrskgvknvvtyttdgckagkvdadmkrgkdtyntsggkagrardvssgdykdhdgdykdhddykddddkmagr

SEQ ID NO: 19 - DNA, 3481 bp, synthetic construct
  misc_difference 1..3481: note = PPR vector
  misc_difference 2858: note = a, c, g, t or u
  misc_difference 2914: note = a, c, g, t or u
  misc_difference 2926: note = a, c, g, t or u
  misc_difference 2934: note = a, c, g, t or u
  misc_difference 2939: note = a, c, g, t or u
  misc_difference 2941: note = a, c, g, t or u
  misc_difference 2944: note = a, c, g, t or u
  misc_difference 2952: note = a, c, g, t or u
  misc_difference 2956: note = a, c, g, t or u
  misc_difference 2960: note = a, c, g, t or u
  misc_difference 2969: note = a, c, g, t or u
  misc_difference 2976: note = a, c, g, t or u
  misc_difference 2986: note = a, c, g, t or u
  misc_difference 3023: note = a, c, g, t or u
  misc_difference 3029: note = a, c, g, t or u
  misc_difference 3031: note = a, c, g, t or u
  misc_difference 3070: note = a, c, g, t or u
  misc_difference 3098: note = a, c, g, t or u
  misc_difference 3139: note = a, c, g, t or u
  misc_difference 3167: note = a, c, g, t or u
  misc_difference 3208: note = a, c, g, t or u
  misc_difference 3236: note = a, c, g, t or u
  misc_difference 3277: note = a, c, g, t or u
  misc_difference 3305: note = a, c, g, t or u
  misc_difference 3346: note = a, c, g, t or u
  misc_difference 3374: note = a, c, g, t or u
  misc_difference 3415: note = a, c, g, t or u
  misc_difference 3441: note = a, c, g, t or u
  source 1..3481: mol_type = other DNA
atggccggagtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagccaaagaaaaagagaaaggttagccatggctccggcggcagcgggggagggctccatatgggaaactccgtggtcacatacaacaccaacatcgaccagctgtgcaagagcggcaagatcgaggaggccctgaagctgttcaaggagatggaggagaagggcatcacccccagcgtggtcacatacaccacactgatcgacggactgtgtaaagccggcgacgtggacgaagccctcgagctgttcaaagagatgcggagcaagggcgtgaagcccaacgtggtgacatacaccaccctgatcgacggcctgtgcaaggccggcaaagtggacgaggccctggagctgttcgacgagatgaaggagaggggcatcaagcccgacgtggtcacatacaacaccctgatcgacggcctgtgcaagagcggcaagatcgaggaggccctgaagctgttcaaggagatggaggagaagggcatcacccccagcgtggttacatacaccacactgatcgacggactgtgtaaagccggcgacgtggacgaagccctcgagctgttcaaagagatgcggagcaagggcgtgaagcccaacgtggtcacatacaccaccctgatcgacggcctgtgcaaggccggcaaggtggatgaggccctggagctgttcgacgagatgaaggagaggggcatcaagcccgacgtggttacatacaacaccctgatcgacggcctgtgcaagagcggcaagatcgaggaggccctgaagctgttcaaggagatggaggagaagggcatcacccccagcgtggtcacatacaccacactgatcgacggactgtgtaaagccggcgacgtggacgaagccctcgagctgttcaaagagatgcggagcaagggcgtgaagcccaacgtggtgacatacaccaccctgatcgacggcctgtgcaaggccggcaaagtggacgaggccctggagctgttcgacgagatgaaggagaggggcatcaagcccgacgtggtcacatacaacaccctgatcgacggcctgtgcaagagcggcaagatcgaggaggccctgaagctgttcaaggagatggaggagaagggcatcacccccagcgtggttacatacaccacactgatcgacggactgtgtaaagccggcgacgtggacgaagccctcgagctgttcaaagagatgcggagcaagggcgtgaagcccaacgtggtcacatacaccaccctgatcgacggcctgtgcaaggccggcaaggtggatgaggccctggagctgttcgacgagatgaaggagaggggcatcaagcccgacgtggtcacatacaacaccctgatcgacggcctgtgcaagagcggcaagatcgaggaggccctgaagctgttcaaggagatggaggagaagggcatcacccccagcgtggtcacatacaccacactgatcgacggactgtgtaaagccggcgacgtggacgaagccctcgagctgttcaaagagatgcggagcaagggcgtgaagcccaacgtggttacatacaccaccctgatcgacggcctgtgcaaggccggcaaagtggacgaggccctggagctgttcgacgagatgaaggagaggggcatcaagcccgacgtggtcacatacaacaccctgatcgacggcctgtgcaagagcggcaagatcgaggaggccctgaagctgttcaaggagatggaggagaagggcatcacccccagcgtggtgacatacaccacactgatcgacggactgtgtaaagccggcgacgtggacgaagccctcgagctgttcaaagagatgcggagcaagggcgtgaagcccaacgtggtcacatacaccaccctgatcgacggcctgtgcaaggccggcaaggtggatgaggccctggagctgttcgacgagatgaaggagaggggcatcaagcccgacgagctgacctacaacaccctgatcagcggcctgggcaaggccggcagagccagagacccccccgtgctcagtagcggggactataaggaccacgacggagactacaaggatcatgatattgattacaaagacgatgacgataagatggccggacgctagmagvskgtgvvvdgdvnghksvsgggdatygktkcttgkvwtvtttygvcsrydhmkhdksamgyvrtkddgnyktravkgdtvnrkgdkdgnghkynynshnvymadkkngkvnkrhndgsvadhyntgdgvdnhystsaskdnkrdhmvvtaagtgmdykkkkrkvshgsggsggghmgnsvvtyntndcksgkakkmkgtsvvtyttdgckagdvdakmrskgvknvvtyttdgckagkvdadmkrgkdvvtyntdgcksgkakkmkgtsvvtyttdgckagdvdakmrskgvknvvtyttdgckagkvdadmkrgkdvvtyntdgcksgkakkmkgtsvvtyttdgckagdvdakmrskgvknvvtyttdgckagkvdadmkrgkdvvtyntdgcksgkakkmkgtsvvtyttdgckagdvdakmrskgvknvvtyttdgckagkvdadmkrgkdvvtyntdgcksgkakkmkgtsvvtyttdgckagdvdakmrskgvknvvtyttdgckagkvdadmkrgkdvvtyntdgcksgkakkmkgtsvvtyttdgckagdvdakmrskgvknvvtyttdgckagkvdadmkrgkdtyntsggkagrardvssgdykdhdgdykdhddykddddkmagr

SEQ ID NO: 20 - DNA, 3481 bp, synthetic construct
  misc_difference 1..3481: note = PPR vector
  misc_difference 2858: note = a, c, g, t or u
  misc_difference 2914: note = a, c, g, t or u
  misc_difference 2926: note = a, c, g, t or u
  misc_difference 2934: note = a, c, g, t or u
  misc_difference 2939: note = a, c, g, t or u
  misc_difference 2941: note = a, c, g, t or u
  misc_difference 2944: note = a, c, g, t or u
  misc_difference 2952: note = a, c, g, t or u
  misc_difference 2956: note = a, c, g, t or u
  misc_difference 2960: note = a, c, g, t or u
  misc_difference 2969: note = a, c, g, t or u
  misc_difference 2976: note = a, c, g, t or u
  misc_difference 2986: note = a, c, g, t or u
  misc_difference 3023: note = a, c, g, t or u
  misc_difference 3029: note = a, c, g, t or u
  misc_difference 3031: note = a, c, g, t or u
  misc_difference 3070: note = a, c, g, t or u
  misc_difference 3098: note = a, c, g, t or u
  misc_difference 3139: note = a, c, g, t or u
  misc_difference 3167: note = a, c, g, t or u
  misc_difference 3208: note = a, c, g, t or u
  misc_difference 3236: note = a, c, g, t or u
  misc_difference 3277: note = a, c, g, t or u
  misc_difference 3305: note = a, c, g, t or u
  misc_difference 3346: note = a, c, g, t or u
  misc_difference 3374: note = a, c, g, t or u
  misc_difference 3415: note = a, c, g, t or u
  misc_difference 3441: note = a, c, g, t or u
  source 1..3481: mol_type = other DNA
atggccggagtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagccaaagaaaaagagaaaggttagccatggctccggcggcagcgggggagggctccatatgggaaactccgtggtcacatacaacaccaacatcgacgaactgtgcaagagcggcaagatcgaggaggccctgaagctgttcaaggagatggaggagaagggcatcacccccagcgtggtcacatacaccacactgatcgacggactgtgtaaagccggcgacgtggacgaagccctcgagctgttcaaagagatgcggagcaagggcgtgaagcccaacgtggtgacatacaccaccctgatcgacggcctgtgcaaggccggcaaagtggacgaggccctggagctgttcgacgagatgaaggagaggggcatcaagcccgacgtggtcacatacaacaccctgatcgacggcctgtgcaagagcggcaagatcgaggaggccctgaagctgttcaaggagatggaggagaagggcatcacccccagcgtggttacatacaccacactgatcgacggactgtgtaaagccggcgacgtggacgaagccctcgagctgttcaaagagatgcggagcaagggcgtgaagcccaacgtggtcacatacaccaccctgatcgacggcctgtgcaaggccggcaaggtggatgaggccctggagctgttcgacgagatgaaggagaggggcatcaagcccgacgtggttacatacaacaccctgatcgacggcctgtgcaagagcggcaagatcgaggaggccctgaagctgttcaaggagatggaggagaagggcatcacccccagcgtggtcacatacaccacactgatcgacggactgtgtaaagccggcgacgtggacgaagccctcgagctgttcaaagagatgcggagcaagggcgtgaagcccaacgtggtgacatacaccaccctgatcgacggcctgtgcaaggccggcaaagtggacgaggccctggagctgttcgacgagatgaaggagaggggcatcaagcccgacgtggtcacatacaacaccctgatcgacggcctgtgcaagagcggcaagatcgaggaggccctgaagctgttcaaggagatggaggagaagggcatcacccccagcgtggttacatacaccacactgatcgacggactgtgtaaagccggcgacgtggacgaagccctcgagctgttcaaagagatgcggagcaagggcgtgaagcccaacgtggtcacatacaccaccctgatcgacggcctgtgcaaggccggcaaggtggatgaggccctggagctgttcgacgagatgaaggagaggggcatcaagcccgacgtggtcacatacaacaccctgatcgacggcctgtgcaagagcggcaagatcgaggaggccctgaagctgttcaaggagatggaggagaagggcatcacccccagcgtggtcacatacaccacactgatcgacggactgtgtaaagccggcgacgtggacgaagccctcgagctgttcaaagagatgcggagcaagggcgtgaagcccaacgtggttacatacaccaccctgatcgacggcctgtgcaaggccggcaaagtggacgaggccctggagctgttcgacgagatgaaggagaggggcatcaagcccgacgtggtcacatacaacaccctgatcgacggcctgtgcaagagcggcaagatcgaggaggccctgaagctgttcaaggagatggaggagaagggcatcacccccagcgtggtgacatacaccacactgatcgacggactgtgtaaagccggcgacgtggacgaagccctcgagctgttcaaagagatgcggagcaagggcgtgaagcccaacgtggtcacatacaccaccctgatcgacggcctgtgcaaggccggcaaggtggatgaggccctggagctgttcgacgagatgaaggagaggggcatcaagcccgacgagctgacctacaacaccctgatcagcggcctgggcaaggccggcagagccagagacccccccgtgctcagtagcggggactataaggaccacgacggagactacaaggatcatgatattgattacaaagacgatgacgataagatggccggacgctagmagvskgtgvvvdgdvnghksvsgggdatygktkcttgkvwtvtttygvcsrydhmkhdksamgyvrtkddgnyktravkgdtvnrkgdkdgnghkynynshnvymadkkngkvnkrhndgsvadhyntgdgvdnhystsaskdnkrdhmvvtaagtgmdykkkkrkvshgsggsggghmgnsvvtyntndcksgkakkmkgtsvvtyttdgckagdvdakmrskgvknvvtyttdgckagkvdadmkrgkdvvtyntdgcksgkakkmkgtsvvtyttdgckagdvdakmrskgvknvvtyttdgckagkvdadmkrgkdvvtyntdgcksgkakkmkgtsvvtyttdgckagdvdakmrskgvknvvtyttdgckagkvdadmkrgkdvvtyntdgcksgkakkmkgtsvvtyttdgckagdvdakmrskgvknvvtyttdgckagkvdadmkrgkdvvtyntdgcksgkakkmkgtsvvtyttdgckagdvdakmrskgvknvvtyttdgckagkvdadmkrgkdvvtyntdgcksgkakkmkgtsvvtyttdgckagdvdakmrskgvknvvtyttdgckagkvdadmkrgkdtyntsggkagrardvssgdykdhdgdykdhddykddddkmagr

SEQ ID NO: 21 - DNA, 3482 bp, synthetic construct
  misc_difference 1..3482: note = PPR vector
  misc_difference 2858: note = a, c, g, t or u
  misc_difference 2914: note = a, c, g, t or u
  misc_difference 2926: note = a, c, g, t or u
  misc_difference 2934: note = a, c, g, t or u
  misc_difference 2939: note = a, c, g, t or u
  misc_difference 2941: note = a, c, g, t or u
  misc_difference 2944: note = a, c, g, t or u
  misc_difference 2952: note = a, c, g, t or u
  misc_difference 2956: note = a, c, g, t or u
  misc_difference 2960: note = a, c, g, t or u
  misc_difference 2969: note = a, c, g, t or u
  misc_difference 2976: note = a, c, g, t or u
  misc_difference 2986: note = a, c, g, t or u
  misc_difference 3023: note = a, c, g, t or u
  misc_difference 3029: note = a, c, g, t or u
  misc_difference 3031: note = a, c, g, t or u
  misc_difference 3071: note = a, c, g, t or u
  misc_difference 3099: note = a, c, g, t or u
  misc_difference 3140: note = a, c, g, t or u
  misc_difference 3168: note = a, c, g, t or u
  misc_difference 3209: note = a, c, g, t or u
  misc_difference 3237: note = a, c, g, t or u
  misc_difference 3278: note = a, c, g, t or u
  misc_difference 3306: note = a, c, g, t or u
  misc_difference 3347: note = a, c, g, t or u
  misc_difference 3375: note = a, c, g, t or u
  misc_difference 3416: note = a, c, g, t or u
  misc_difference 3442: note = a, c, g, t or u
  source 1..3482: mol_type = other DNA
atggccggagtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagccaaagaaaaagagaaaggttagccatggctccggcggcagcgggggagggctccatatgggaaactccgtggtcacatacaacaccaacatcgacaaactgtgcaagagcggcaagatcgaggaggccctgaagctgttcaaggagatggaggagaagggcatcacccccagcgtggtcacatacaccacactgatcgacggactgtgtaaagccggcgacgtggacgaagccctcgagctgttcaaagagatgcggagcaagggcgtgaagcccaacgtggtgacatacaccaccctgatcgacggcctgtgcaaggccggcaaagtggacgaggccctggagctgttcgacgagatgaaggagaggggcatcaagcccgacgtggtcacatacaacaccctgatcgacggcctgtgcaagagcggcaagatcgaggaggccctgaagctgttcaaggagatggaggagaagggcatcacccccagcgtggttacatacaccacactgatcgacggactgtgtaaagccggcgacgtggacgaagccctcgagctgttcaaagagatgcggagcaagggcgtgaagcccaacgtggtcacatacaccaccctgatcgacggcctgtgcaaggccggcaaggtggatgaggccctggagctgttcgacgagatgaaggagaggggcatcaagcccgacgtggttacatacaacaccctgatcgacggcctgtgcaagagcggcaagatcgaggaggccctgaagctgttcaaggagatggaggagaagggcatcacccccagcgtggtcacatacaccacactgatcgacggactgtgtaaagccggcgacgtggacgaagccctcgagctgttcaaagagatgcggagcaagggcgtgaagcccaacgtggtgacatacaccaccctgatcgacggcctgtgcaaggccggcaaagtggacgaggccctggagctgttcgacgagatgaaggagaggggcatcaagcccgacgtggtcacatacaacaccctgatcgacggcctgtgcaagagcggcaagatcgaggaggccctgaagctgttcaaggagatggaggagaagggcatcacccccagcgtggttacatacaccacactgatcgacggactgtgtaaagccggcgacgtggacgaagccctcgagctgttcaaagagatgcggagcaagggcgtgaagcccaacgtggtcacatacaccaccctgatcgacggcctgtgcaaggccggcaaggtggatgaggccctggagctgttcgacgagatgaaggagaggggcatcaagcccgacgtggtcacatacaacaccctgatcgacggcctgtgcaagagcggcaagatcgaggaggccctgaagctgttcaaggagatggaggagaagggcatcacccccagcgtggtcacatacaccacactgatcgacggactgtgtaaagccggcgacgtggacgaagccctcgagctgttcaaagagatgcggagcaagggcgtgaagcccaacgtggttacatacaccaccctgatcgacggcctgtgcaaggccggcaaagtggacgaggccctggagctgttcgacgagatgaaggagaggggcatcaagcccgacgtggtcacatacaacaccctgatcgacggcctgtgcaagagcggcaagatcgaggaggccctgaagctgttcaaggagatggaggagaagggcatcacccccagcgtggtgacatacaccacactgatcgacggactgtgtaaagccggcgacgtggacgaagccctcgagctgttcaaagagatgcggagcaagggcgtgaagcccaacgtggtcacatacaccaccctgatcgacggcctgtgcaaggccggcaaggtggatgaggccctggagctgttcgacgagatgaaggagaggggcatcaagcccgacgagctgacctacaacaccctgatcagcggcctgggcaaggccggcagagccagagacccccccgtgctcagtagcggggactataaggaccacgacggagactacaaggatcatgatattgattacaaagacgatgacgataagatggccggacgctagmagvskgtgvvvdgdvnghksvsgggdatygktkcttgkvwtvtttygvcsrydhmkhdksamgyvrtkddgnyktravkgdtvnrkgdkdgnghkynynshnvymadkkngkvnkrhndgsvadhyntgdgvdnhystsaskdnkrdhmvvtaagtgmdykkkkrkvshgsggsggghmgnsvvtyntndkcksgkakkmkgtsvvtyttdgckagdvdakmrskgvknvvtyttdgckagkvdadmkrgkdvvtyntdgcksgkakkmkgtsvvtyttdgckagdvdakmrskgvknvvtyttdgckagkvdadmkrgkdvvtyntdgcksgkakkmkgtsvvtyttdgckagdvdakmrskgvknvvtyttdgckagkvdadmkrgkdvvtyntdgcksgkakkmkgtsvvtyttdgckagdvdakmrskgvknvvtyttdgckagkvdadmkrgkdvvtyntdgcksgkakkmkgtsvvtyttdgckagdvdakmrskgvknvvtyttdgckagkvdadmkrgkdvvtyntdgcksgkakkmkgtsvvtyttdgckagdvdakmrskgvknvvtyttdgckagkvdadmkrgkdtyntsggkagrardvssgdykdhdgdykdhddykddddkmagr

SEQ ID NO: 22 - DNA, 3482 bp, synthetic construct
  misc_difference 1..3482: note = PPR vector
  misc_difference 2858: note = a, c, g, t or u
  misc_difference 2914: note = a, c, g, t or u
  misc_difference 2926: note = a, c, g, t or u
  misc_difference 2934: note = a, c, g, t or u
  misc_difference 2939: note = a, c, g, t or u
  misc_difference 2941: note = a, c, g, t or u
  misc_difference 2944: note = a, c, g, t or u
  misc_difference 2952: note = a, c, g, t or u
  misc_difference 2956: note = a, c, g, t or u
  misc_difference 2960: note = a, c, g, t or u
  misc_difference 2969: note = a, c, g, t or u
  misc_difference 2976: note = a, c, g, t or u
  misc_difference 2986: note = a, c, g, t or u
  misc_difference 3023: note = a, c, g, t or u
  misc_difference 3029: note = a, c, g, t or u
  misc_difference 3071: note = a, c, g, t or u
  misc_difference 3099: note = a, c, g, t or u
  misc_difference 3140: note = a, c, g, t or u
  misc_difference 3168: note = a, c, g, t or u
  misc_difference 3209: note = a, c, g, t or u
  misc_difference 3237: note = a, c, g, t or u
  misc_difference 3278: note = a, c, g, t or u
  misc_difference 3306: note = a, c, g, t or u
  misc_difference 3347: note = a, c, g, t or u
  misc_difference 3375: note = a, c, g, t or u
  misc_difference 3416: note = a, c, g, t or u
  misc_difference 3442: note = a, c, g, t or u
  source 1..3482: mol_type = other DNA
atggccggagtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagccaaagaaaaagagaaaggttagccatggctccggcggcagcgggggagggctccatatgggaaactccgtggtcacatacaacaccgatatcgacggcctgtgcaagagcggcaagatcgaggaggccctgaagctgttcaaggagatggaggagaagggcatcacccccagcgtggtcacatacaccacactgatcgacggactgtgtaaagccggcgacgtggacgaagccctcgagctgttcaaagagatgcggagcaagggcgtgaagcccaacgtggtgacatacaccaccctgatcgacggcctgtgcaaggccggcaaagtggacgaggccctggagctgttcgacgagatgaaggagaggggcatcaagcccgacgtggtcacatacaacaccctgatcgacggcctgtgcaagagcggcaagatcgaggaggccctgaagctgttcaaggagatggaggagaagggcatcacccccagcgtggttacatacaccacactgatcgacggactgtgtaaagccggcgacgtggacgaagccctcgagctgttcaaagagatgcggagcaagggcgtgaagcccaacgtggtcacatacaccaccctgatcgacggcctgtgcaaggccggcaaggtggatgaggccctggagctgttcgacgagatgaaggagaggggcatcaagcccgacgtggttacatacaacaccctgatcgacggcctgtgcaagagcggcaagatcgaggaggccctgaagctgttcaaggagatggaggagaagggcatcacccccagcgtggtcacatacaccacactgatcgacggactgtgtaaagccggcgacgtggacgaagccctcgagctgttcaaagagatgcggagcaagggcgtgaagcccaacgtggtgacatacaccaccctgatcgacggcctgtgcaaggccggcaaagtggacgaggccctggagctgttcgacgagatgaaggagaggggcatcaagcccgacgtggtcacatacaacaccctgatcgacggcctgtgcaagagcggcaagatcgaggaggccctgaagctgttcaaggagatggaggagaagggcatcacccccagcgtggttacatacaccacactgatcgacggactgtgtaaagccggcgacgtggacgaagccctcgagctgttcaaagagatgcggagcaagggcgtgaagcccaacgtggtcacatacaccaccctgatcgacggcctgtgcaaggccggcaaggtggatgaggccctggagctgttcgacgagatgaaggagaggggcatcaagcccgacgtggtcacatacaacaccctgatcgacggcctgtgcaagagcggcaagatcgaggaggccctgaagctgttcaaggagatggaggagaagggcatcacccccagcgtggtcacatacaccacactgatcgacggactgtgtaaagccggcgacgtggacgaagccctcgagctgttcaaagagatgcggagcaagggcgtgaagcccaacgtggttacatacaccaccctgatcgacggcctgtgcaaggccggcaaagtggacgaggccctggagctgttcgacgagatgaaggagaggggcatcaagcccgacgtggtcacatacaacaccctgatcgacggcctgtgcaagagcggcaagatcgaggaggccctgaagctgttcaaggagatggaggagaagggcatcacccccagcgtggtgacatacaccacactgatcgacggactgtgtaaagccggcgacgtggacgaagccctcgagctgttcaaagagatgcggagcaagggcgtgaagcccaacgtggtcacatacaccaccctgatcgacggcctgtgcaaggccggcaaggtggatgaggccctggagctgttcgacgagatgaaggagaggggcatcaagcccgacgagctgacctacaacaccctgatcagcggcctgggcaaggccggcagagccagagacccccccgtgctcagtagcggggactataaggaccacgacggagactacaaggatcatgatattgattacaaagacgatgacgataagatggccggacgctagmagvskgtgvvvdgdvnghksvsgggdatygktkcttgkvwtvtttygvcsrydhmkhdksamgyvrtkddgnyktravkgdtvnrkgdkdgnghkynynshnvymadkkngkvnkrhndgsvadhyntgdgvdnhystsaskdnkrdhmvvtaagtgmdykkkkrkvshgsggsggghmgnsvvtyntddgcksgkakkmkgtsvvtyttdgckagdvdakmrskgvknvvtyttdgckagkvdadmkrgkdvvtyntdgcksgkakkmkgtsvvtyttdgckagdvdakmrskgvknvvtyttdgckagkvdadmkrgkdvvtyntdgcksgkakkmkgtsvvtyttdgckagdvdakmrskgvknvvtyttdgckagkvdadmkrgkdvvtyntdgcksgkakkmkgtsvvtyttdgckagdvdakmrskgvknvvtyttdgckagkvdadmkrgkdvvtyntdgcksgkakkmkgtsvvtyttdgckagdvdakmrskgvknvvtyttdgckagkvdadmkrgkdvvtyntdgcksgkakkmkgtsvvtyttdgckagdvdakmrskgvknvvtyttdgckagkvdadmkrgkdtyntsggkagrardvssgdykdhdgdykdhddykddddkmagr

SEQ ID NO: 23 - DNA, 3551 bp, synthetic construct
  misc_difference 1..3551: note = PPR vector
  misc_difference 2912: note = a, c, g, t or u
  misc_difference 2925: note = a, c, g, t or u
  misc_difference 2982: note = a, c, g, t or u
  misc_difference 2990: note = a, c, g, t or u
  misc_difference 2995..2996: note = a, c, g, t or u
  misc_difference 3007: note = a, c, g, t or u
  misc_difference 3011: note = a, c, g, t or u
  misc_difference 3015: note = a, c, g, t or u
  misc_difference 3025: note = a, c, g, t or u
  misc_difference 3032: note = a, c, g, t or u
  misc_difference 3042: note = a, c, g, t or u
  misc_difference 3066: note = a, c, g, t or u
  misc_difference 3072: note = a, c, g, t or u
  misc_difference 3113: note = a, c, g, t or u
  misc_difference 3141: note = a, c, g, t or u
  misc_difference 3182: note = a, c, g, t or u
  misc_difference 3210: note = a, c, g, t or u
  misc_difference 3251: note = a, c, g, t or u
  misc_difference 3279: note = a, c, g, t or u
  misc_difference 3320: note = a, c, g, t or u
  misc_difference 3348: note = a, c, g, t or u
  misc_difference 3389: note = a, c, g, t or u
  misc_difference 3417: note = a, c, g, t or u
  misc_difference 3458: note = a, c, g, t or u
  misc_difference 3484: note = a, c, g, t or u
  source 1..3551: mol_type = other DNA
atggccggagtgtccaaaggcgaggagctgtttaccggcgtcgtgcctattctggtggagctggacggcgacgtgaacggccacaagttctccgtgaggggcgagggcgaaggcgatgccacaaacggcaagctgaccctcaagttcatctgcaccactggtaaactgcccgttccttggcccacactggtgaccaccttcggctacggcgtggcttgtttctctcgttaccccgaccatatgaagcagcacgacttcttcaagtccgccatgcccgagggatacgtgcaagaaaggaccatctccttcaaggacgatggcacctacaagaccagagccgaggtgaagttcgagggcgacacactggtgaatcgtatcgaactgaagggcatcgacttcaaagaggacggcaacattctgggccacaagctggagtacaacttcaacagccactacgtgtacatcaccgccgataagcagaagaactgcatcaaggccaacttcaagattcgtcacaacgtggaggatggctccgtgcagctggccgatcactaccagcagaacacacccatcggcgatggacccgttttactgcccgacaaccactatttaagccaccagagcaagctgtccaaggaccccaacgagaagcgtgatcatatggtgctgctcgagtttgtgaccgccgccggcatcacccatggaatggacgagctgtacaagagccggctccatatgggaaactccgtggtcacatacaacaccctgatcgacggcctgtgcaagagcggcaagatcgaggaggccctgaagctgttcaaggagatggaggagaagggcatcacccccagcgtggtcacatacaccacactgatcgacggactgtgtaaagccggcgacgtggacgaagccctcgagctgttcaaagagatgcggagcaagggcgtgaagcccaacgtggtgacatacaccaccctgatcgacggcctgtgcaaggccggcaaagtggacgaggccctggagctgttcgacgagatgaaggagaggggcatcaagcccgacgtggtcacatacaacaccctgatcgacggcctgtgcaagagcggcaagatcgaggaggccctgaagctgttcaaggagatggaggagaagggcatcacccccagcgtggttacatacaccacactgatcgacggactgtgtaaagccggcgacgtggacgaagccctcgagctgttcaaagagatgcggagcaagggcgtgaagcccaacgtggtcacatacaccaccctgatcgacggcctgtgcaaggccggcaaggtggatgaggccctggagctgttcgacgagatgaaggagaggggcatcaagcccgacgtggttacatacaacaccctgatcgacggcctgtgcaagagcggcaagatcgaggaggccctgaagctgttcaaggagatggaggagaagggcatcacccccagcgtggtcacatacaccacactgatcgacggactgtgtaaagccggcgacgtggacgaagccctcgagctgttcaaagagatgcggagcaagggcgtgaagcccaacgtggtgacatacaccaccctgatcgacggcctgtgcaaggccggcaaagtggacgaggccctggagctgttcgacgagatgaaggagaggggcatcaagcccgacgtggtcacatacaacaccctgatcgacggcctgtgcaagagcggcaagatcgaggaggccctgaagctgttcaaggagatggaggagaagggcatcacccccagcgtggttacatacaccacactgatcgacggactgtgtaaagccggcgacgtggacgaagccctcgagctgttcaaagagatgcggagcaagggcgtgaagcccaacgtggtcacatacaccaccctgatcgacggcctgtgcaaggccggcaaggtggatgaggccctggagctgttcgacgagatgaaggagaggggcatcaagcccgacgtggtcacatacaacaccctgatcgacggcctgtgcaagagcggcaagatcgaggaggccctgaagctgttcaaggagatggaggagaagggcatcacccccagcgtggtcacatacaccacactgatcgacggactgtgtaaagccggcgacgtggacgaagccctcgagctgttcaaagagatgcggagcaagggcgtgaagcccaacgtggttacatacaccaccctgatcgacggcctgtgcaaggccggcaaagtggacgaggccctggagctgttcgacgagatgaaggagaggggcatcaagcccgacgtggtcacatacaacaccctgatcgacggcctgtgcaagagcggcaagatcgaggaggccctgaagctgttcaaggagatggaggagaagggcatcacccccagcgtggtgacatacaccacactgatcgacggactgtgtaaagccggcgacgtggacgaagccctcgagctgttcaaagagatgcggagcaagggcgtgaagcccaacgtggtcacatacaccaccctgatcgacggcctgtgcaaggccggcaaggtggatgaggccctggagctgttcgacgagatgaaggagaggggcatcaagcccgacgagctgacctacaacaccctgatcagcggcctgggcaaggccggcagagccagagacccccccgtgctcagtagccccaagaagaaacgcaaagtcgaggatccaaagaagaaaaggaaggttgaagaccccaagaaaaagaggaaggtgggttccgactataaggaccacgacggagactacaaggatcatgatattgattacaaagacgatgacgataagatggccccaaagaagaagcggaaggtcggacgctagmagvskgtgvvvdgdvnghksvrgggdatngktkcttgkvwtvttgygvacsrydhmkhdksamgyvrtskddgtyktravkgdtvnrkgdkdgnghkynnshyvytadkknckankrhnvdgsvadhyntgdgvdnhyshskskdnkrdhmvvtaagthgmdyksrhmgnsvvtyntdgcksgkakkmkgtsvvtyttdgckagdvdakmrskgvknvvtyttdgckagkvdadmkrgkdvvtyntdgcksgkakkmkgtsvvtyttdgckagdvdakmrskgvknvvtyttdgckagkvdadmkrgkdvvtyntdgcksgkakkmkgtsvvtyttdgckagdvdakmrskgvknvvtyttdgckagkvdadmkrgkdvvtyntdgcksgkakkmkgtsvvtyttdgckagdvdakmrskgvknvvtyttdgckagkvdadmkrgkdvvtyntdgcksgkakkmkgtsvvtyttdgckagdvdakmrskgvknvvtyttdgckagkvdadmkrgkdvvtyntdgcksgkakkmkgtsvvtyttdgckagdvdakmrskgvknvvtyttdgckagkvdadmkrgkdtyntsggkagrardvsskkkrkvdkkkrkvdkkkrkvgsdykdhdgdykdhddykddddkmakkkrkvgr

SEQ ID NO: 24 - DNA, 3550 bp, synthetic construct
  misc_difference 1..3550: note = PPR vector
  misc_difference 2912: note = a, c, g, t or u
  misc_difference 2925: note = a, c, g, t or u
  misc_difference 2982: note = a, c, g, t or u
  misc_difference 2990: note = a, c, g, t or u
  misc_difference 2995..2996: note = a, c, g, t or u
  misc_difference 3007: note = a, c, g, t or u
  misc_difference 3011: note = a, c, g, t or u
  misc_difference 3015: note = a, c, g, t or u
  misc_difference 3025: note = a, c, g, t or u
  misc_difference 3032: note = a, c, g, t or u
  misc_difference 3042: note = a, c, g, t or u
  misc_difference 3066: note = a, c, g, t or u
  misc_difference 3072: note = a, c, g, t or u
  misc_difference 3112: note = a, c, g, t or u
  misc_difference 3140: note = a, c, g, t or u
  misc_difference 3181: note = a, c, g, t or u
  misc_difference 3209: note = a, c, g, t or u
  misc_difference 3250: note = a, c, g, t or u
  misc_difference 3278: note = a, c, g, t or u
  misc_difference 3319: note = a, c, g, t or u
  misc_difference 3347: note = a, c, g, t or u
  misc_difference 3388: note = a, c, g, t or u
  misc_difference 3416: note = a, c, g, t or u
  misc_difference 3457: note = a, c, g, t or u
  misc_difference 3483: note = a, c, g, t or u
  source 1..3550: mol_type = other DNA
atggccggagtgtccaaaggcgaggagctgtttaccggcgtcgtgcctattctggtggagctggacggcgacgtgaacggccacaagttctccgtgaggggcgagggcgaaggcgatgccacaaacggcaagctgaccctcaagttcatctgcaccactggtaaactgcccgttccttggcccacactggtgaccaccttcggctacggcgtggcttgtttctctcgttaccccgaccatatgaagcagcacgacttcttcaagtccgccatgcccgagggatacgtgcaagaaaggaccatctccttcaaggacgatggcacctacaagaccagagccgaggtgaagttcgagggcgacacactggtgaatcgtatcgaactgaagggcatcgacttcaaagaggacggcaacattctgggccacaagctggagtacaacttcaacagccactacgtgtacatcaccgccgataagcagaagaactgcatcaaggccaacttcaagattcgtcacaacgtggaggatggctccgtgcagctggccgatcactaccagcagaacacacccatcggcgatggacccgttttactgcccgacaaccactatttaagccaccagagcaagctgtccaaggaccccaacgagaagcgtgatcatatggtgctgctcgagtttgtgaccgccgccggcatcacccatggaatggacgagctgtacaagagccggctccatatgggaaactccgtggtcacatacaacaccctgatcgacgaactgtgcaagagcggcaagatcgaggaggccctgaagctgttcaaggagatggaggagaagggcatcacccccagcgtggtcacatacaccacactgatcgacggactgtgtaaagccggcgacgtggacgaagccctcgagctgttcaaagagatgcggagcaagggcgtgaagcccaacgtggtgacatacaccaccctgatcgacggcctgtgcaaggccggcaaagtggacgaggccctggagctgttcgacgagatgaaggagaggggcatcaagcccgacgtggtcacatacaacaccctgatcgacggcctgtgcaagagcggcaagatcgaggaggccctgaagctgttcaaggagatggaggagaagggcatcacccccagcgtggttacatacaccacactgatcgacggactgtgtaaagccggcgacgtggacgaagccctcgagctgttcaaagagatgcggagcaagggcgtgaagcccaacgtggtcacatacaccaccctgatcgacggcctgtgcaaggccggcaaggtggatgaggccctggagctgttcgacgagatgaaggagaggggcatcaagcccgacgtggttacatacaacaccctgatcgacggcctgtgcaagagcggcaagatcgaggaggccctgaagctgttcaaggagatggaggagaagggcatcacccccagcgtggtcacatacaccacactgatcgacggactgtgtaaagccggcgacgtggacgaagccctcgagctgttcaaagagatgcggagcaagggcgtgaagcccaacgtggtgacatacaccaccctgatcgacggcctgtgcaaggccggcaaagtggacgaggccctggagctgttcgacgagatgaaggagaggggcatcaagcccgacgtggtcacatacaacaccctgatcgacggcctgtgcaagagcggcaagatcgaggaggccctgaagctgttcaaggagatggaggagaagggcatcacccccagcgtggttacatacaccacactgatcgacggactgtgtaaagccggcgacgtggacgaagccctcgagctgttcaaagagatgcggagcaagggcgtgaagcccaacgtggtcacatacaccaccctgatcgacggcctgtgcaaggccggcaaggtggatgaggccctggagctgttcgacgagatgaaggagaggggcatcaagcccgacgtggtcacatacaacaccctgatcgacggcctgtgcaagagcggcaagatcgaggaggccctgaagctgttcaaggagatggaggagaagggcatcacccccagcgtggtcacatacaccacactgatcgacggactgtgtaaagccggcgacgtggacgaagccctcgagctgttcaaagagatgcggagcaagggcgtgaagcccaacgtggttacatacaccaccctgatcgacggcctgtgcaaggccggcaaagtggacgaggccctggagctgttcgacgagatgaaggagaggggcatcaagcccgacgtggtcacatacaacaccctgatcgacggcctgtgcaagagcggcaagatcgaggaggccctgaagctgttcaaggagatggaggagaagggcatcacccccagcgtggtgacatacaccacactgatcgacggactgtgtaaagccggcgacgtggacgaagccctcgagctgttcaaagagatgcggagcaagggcgtgaagcccaacgtggtcacatacaccaccctgatcgacggcctgtgcaaggccggcaaggtggatgaggccctggagctgttcgacgagatgaaggagaggggcatcaagcccgacgagctgacctacaacaccctgatcagcggcctgggcaaggccggcagagccagagacccccccgtgctcagtagccccaagaagaaacgcaaagtcgaggatccaaagaagaaaaggaaggttgaagaccccaagaaaaagaggaaggtgggttccgactataaggaccacgacggagactacaaggatcatgatattgattacaaagacgatgacgataagatggccccaaagaagaagcggaaggtcggacgctagmagvskgtgvvvdgdvnghksvrgggdatngktkcttgkvwtvttgygvacsrydhmkhdksamgyvrtskddgtyktravkgdtvnrkgdkdgnghkynnshyvytadkknckankrhnvdgsvadhyntgdgvdnhyshskskdnkrdhmvvtaagthgmdyksrhmgnsvvtyntdcksgkakkmkgtsvvtyttdgckagdvdakmrskgvknvvtyttdgckagkvdadmkrgkdvvtyntdgcksgkakkmkgtsvvtyttdgckagdvdakmrskgvknvvtyttdgckagkvdadmkrgkdvvtyntdgcksgkakkmkgtsvvtyttdgckagdvdakmrskgvknvvtyttdgckagkvdadmkrgkdvvtyntdgcksgkakkmkgtsvvtyttdgckagdvdakmrskgvknvvtyttdgckagkvdadmkrgkdvvtyntdgcksgkakkmkgtsvvtyttdgckagdvdakmrskgvknvvtyttdgckagkvdadmkrgkdvvtyntdgcksgkakkmkgtsvvtyttdgckagdvdakmrskgvknvvtyttdgckagkvdadmkrgkdtyntsggkagrardvsskkkrkvdkkkrkvdkkkrkvgsdykdhdgdykdhddykddddkmakkkrkvgr

SEQ ID NO: 25 - DNA, 3551 bp, synthetic construct
  misc_difference 1..3551: note = PPR vector
  misc_difference 2912: note = a, c, g, t or u
  misc_difference 2925: note = a, c, g, t or u
  misc_difference 2982: note = a, c, g, t or u
  misc_difference 2990: note = a, c, g, t or u
  misc_difference 2995..2996: note = a, c, g, t or u
  misc_difference 3007: note = a, c, g, t or u
  misc_difference 3011: note = a, c, g, t or u
  misc_difference 3015: note = a, c, g, t or u
  misc_difference 3025: note = a, c, g, t or u
  misc_difference 3032: note = a, c, g, t or u
  misc_difference 3042: note = a, c, g, t or u
  misc_difference 3066: note = a, c, g, t or u
  misc_difference 3072: note = a, c, g, t or u
  misc_difference 3074: note = a, c, g, t or u
  misc_difference 3113: note = a, c, g, t or u
  misc_difference 3141: note = a, c, g, t or u
  misc_difference 3182: note = a, c, g, t or u
  misc_difference 3210: note = a, c, g, t or u
  misc_difference 3251: note = a, c, g, t or u
  misc_difference 3279: note = a, c, g, t or u
  misc_difference 3320: note = a, c, g, t or u
  misc_difference 3348: note = a, c, g, t or u
  misc_difference 3389: note = a, c, g, t or u
  misc_difference 3417: note = a, c, g, t or u
  misc_difference 3458: note = a, c, g, t or u
  misc_difference 3484: note = a, c, g, t or u
  source 1..3551: mol_type = other DNA
atggccggagtgtccaaaggcgaggagctgtttaccggcgtcgtgcctattctggtggagctggacggcgacgtgaacggccacaagttctccgtgaggggcgagggcgaaggcgatgccacaaacggcaagctgaccctcaagttcatctgcaccactggtaaactgcccgttccttggcccacactggtgaccaccttcggctacggcgtggcttgtttctctcgttaccccgaccatatgaagcagcacgacttcttcaagtccgccatgcccgagggatacgtgcaagaaaggaccatctccttcaaggacgatggcacctacaagaccagagccgaggtgaagttcgagggcgacacactggtgaatcgtatcgaactgaagggcatcgacttcaaagaggacggcaacattctgggccacaagctggagtacaacttcaacagccactacgtgtacatcaccgccgataagcagaagaactgcatcaaggccaacttcaagattcgtcacaacgtggaggatggctccgtgcagctggccgatcactaccagcagaacacacccatcggcgatggacccgttttactgcccgacaaccactatttaagccaccagagcaagctgtccaaggaccccaacgagaagcgtgatcatatggtgctgctcgagtttgtgaccgccgccggcatcacccatggaatggacgagctgtacaagagccggctccatatgggaaactccgtggtcacatacaacaccaacatcgaccagctgtgcaagagcggcaagatcgaggaggccctgaagctgttcaaggagatggaggagaagggcatcacccccagcgtggtcacatacaccacactgatcgacggactgtgtaaagccggcgacgtggacgaagccctcgagctgttcaaagagatgcggagcaagggcgtgaagcccaacgtggtgacatacaccaccctgatcgacggcctgtgcaaggccggcaaagtggacgaggccctggagctgttcgacgagatgaaggagaggggcatcaagcccgacgtggtcacatacaacaccctgatcgacggcctgtgcaagagcggcaagatcgaggaggccctgaagctgttcaaggagatggaggagaagggcatcacccccagcgtggttacatacaccacactgatcgacggactgtgtaaagccggcgacgtggacgaagccctcgagctgttcaaagagatgcggagcaagggcgtgaagcccaacgtggtcacatacaccaccctgatcgacggcctgtgcaaggccggcaaggtggatgaggccctggagctgttcgacgagatgaaggagaggggcatcaagcccgacgtggttacatacaacaccctgatcgacggcctgtgcaagagcggcaagatcgaggaggccctgaagctgttcaaggagatggaggagaagggcatcacccccagcgtggtcacatacaccacactgatcgacggactgtgtaaagccggcgacgtggacgaagccctcgagctgttcaaagagatgcggagcaagggcgtgaagcccaacgtggtgacatacaccaccctgatcgacggcctgtgcaaggccggcaaagtggacgaggccctggagctgttcgacgagatgaaggagaggggcatcaagcccgacgtggtcacatacaacaccctgatcgacggcctgtgcaagagcggcaagatcgaggaggccctgaagctgttcaaggagatggaggagaagggcatcacccccagcgtggttacatacaccacactgatcgacggactgtgtaaagccggcgacgtggacgaagccctcgagctgttcaaagagatgcggagcaagggcgtgaagcccaacgtggtcacatacaccaccctgatcgacggcctgtgcaaggccggcaaggtggatgaggccctggagctgttcgacgagatgaaggagaggggcatcaagcccgacgtggtcacatacaacaccctgatcgacggcctgtgcaagagcggcaagatcgaggaggccctgaagctgttcaaggagatggaggagaagggcatcacccccagcgtggtcacatacaccacactgatcgacggactgtgtaaagccggcgacgtggacgaagccctcgagctgttcaaagagatgcggagcaagggcgtgaagcccaacgtggttacatacaccaccctgatcgacggcctgtgcaaggccggcaaagtggacgaggccctggagctgttcgacgagatgaaggagaggggcatcaagcccgacgtggtcacatacaacaccctgatcgacggcctgtgcaagagcggcaagatcgaggaggccctgaagctgttcaaggagatggaggagaagggcatcacccccagcgtggtgacatacaccacactgatcgacggactgtgtaaagccggcgacgtggacgaagccctcgagctgttcaaagagatgcggagcaagggcgtgaagcccaacgtggtcacatacaccaccctgatcgacggcctgtgcaaggccggcaaggtggatgaggccctggagctgttcgacgagatgaaggagaggggcatcaagcccgacgagctgacctacaacaccctgatcagcggcctgggcaaggccggcagagccagagacccccccgtgctcagtagccccaagaagaaacgcaaagtcgaggatccaaagaagaaaaggaaggttgaagaccccaagaaaaagaggaaggtgggttccgactataaggaccacgacggagactacaaggatcatgatattgattacaaagacgatgacgataagatggccccaaagaagaagcggaaggtcggacgctagmagvskgtgvvvdgdvnghksvrgggdatngktkcttgkvwtvttgygvacsrydhmkhdksamgyvrtskddgtyktravkgdtvnrkgdkdgnghkynnshyvytadkknckankrhnvdgsvadhyntgdgvdnhyshskskdnkrdhmvvtaagthgmdyksrhmgnsvvtyntndcksgkakkmkgtsvvtyttdgckagdvdakmrskgvknvvtyttdgckagkvdadmkrgkdvvtyntdgcksgkakkmkgtsvvtyttdgckagdvdakmrskgvknvvtyttdgckagkvdadmkrgkdvvtyntdgcksgkakkmkgtsvvtyttdgckagdvdakmrskgvknvvtyttdgckagkvdadmkrgkdvvtyntdgcksgkakkmkgtsvvtyttdgckagdvdakmrskgvknvvtyttdgckagkvdadmkrgkdvvtyntdgcksgkakkmkgtsvvtyttdgckagdvdakmrskgvknvvtyttdgckagkvdadmkrgkdvvtyntdgcksgkakkmkgtsvvtyttdgckagdvdakmrskgvknvvtyttdgckagkvdadmkrgkdtyntsggkagrardvsskkkrkvdkkkrkvdkkkrkvgsdykdhdgdykdhddykddddkmakkkrkvgr

SEQ ID NO: 26 - DNA, 3551 bp, synthetic construct
  misc_difference 1..3551: note = PPR vector
  misc_difference 2912: note = a, c, g, t or u
  misc_difference 2925: note = a, c, g, t or u
  misc_difference 2982: note = a, c, g, t or u
  misc_difference 2990: note = a, c, g, t or u
  misc_difference 2995..2996: note = a, c, g, t or u
  misc_difference 3007: note = a, c, g, t or u
  misc_difference 3011: note = a, c, g, t or u
  misc_difference 3015: note = a, c, g, t or u
  misc_difference 3025: note = a, c, g, t or u
  misc_difference 3032: note = a, c, g, t or u
  misc_difference 3042: note = a, c, g, t or u
  misc_difference 3066: note = a, c, g, t or u
  misc_difference 3072: note = a, c, g, t or u
  misc_difference 3074: note = a, c, g, t or u
  misc_difference 3113: note = a, c, g, t or u
  misc_difference 3141: note = a, c, g, t or u
  misc_difference 3182: note = a, c, g, t or u
  misc_difference 3210: note = a, c, g, t or u
  misc_difference 3251: note = a, c, g, t or u
  misc_difference 3279: note = a, c, g, t or u
  misc_difference 3320: note = a, c, g, t or u
  misc_difference 3348: note = a, c, g, t or u
  misc_difference 3389: note = a, c, g, t or u
  misc_difference 3417: note = a, c, g, t or u
  misc_difference 3458: note = a, c, g, t or u
  misc_difference 3484: note = a, c, g, t or u
  source 1..3551: mol_type = other DNA
atggccggagtgtccaaaggcgaggagctgtttaccggcgtcgtgcctattctggtggagctggacggcgacgtgaacggccacaagttctccgtgaggggcgagggcgaaggcgatgccacaaacggcaagctgaccctcaagttcatctgcaccactggtaaactgcccgttccttggcccacactggtgaccaccttcggctacggcgtggcttgtttctctcgttaccccgaccatatgaagcagcacgacttcttcaagtccgccatgcccgagggatacgtgcaagaaaggaccatctccttcaaggacgatggcacctacaagaccagagccgaggtgaagttcgagggcgacacactggtgaatcgtatcgaactgaagggcatcgacttcaaagaggacggcaacattctgggccacaagctggagtacaacttcaacagccactacgtgtacatcaccgccgataagcagaagaactgcatcaaggccaacttcaagattcgtcacaacgtggaggatggctccgtgcagctggccgatcactaccagcagaacacacccatcggcgatggacccgttttactgcccgacaaccactatttaagccaccagagcaagctgtccaaggaccccaacgagaagcgtgatcatatggtgctgctcgagtttgtgaccgccgccggcatcacccatggaatggacgagctgtacaagagccggctccatatgggaaactccgtggtcacatacaacaccaacatcgacgaactgtgcaagagcggcaagatcgaggaggccctgaagctgttcaaggagatggaggagaagggcatcacccccagcgtggtcacatacaccacactgatcgacggactgtgtaaagccggcgacgtggacgaagccctcgagctgttcaaagagatgcggagcaagggcgtgaagcccaacgtggtgacatacaccaccctgatcgacggcctgtgcaaggccggcaaagtggacgaggccctggagctgttcgacgagatgaaggagaggggcatcaagcccgacgtggtcacatacaacaccctgatcgacggcctgtgcaagagcggcaagatcgaggaggccctgaagctgttcaaggagatggaggagaagggcatcacccccagcgtggttacatacaccacactgatcgacggactgtgtaaagccggcgacgtggacgaagccctcgagctgttcaaagagatgcggagcaagggcgtgaagcccaacgtggtcacatacaccaccctgatcgacggcctgtgcaaggccggcaaggtggatgaggccctggagctgttcgacgagatgaaggagaggggcatcaagcccgacgtggttacatacaacaccctgatcgacggcctgtgcaagagcggcaagatcgaggaggccctgaagctgttcaaggagatggaggagaagggcatcacccccagcgtggtcacatacaccacactgatcgacggactgtgtaaagccggcgacgtggacgaagccctcgagctgttcaaagagatgcggagcaagggcgtgaagcccaacgtggtgacatacaccaccctgatcgacggcctgtgcaaggccggcaaagtggacgaggccctggagctgttcgacgagatgaaggagaggggcatcaagcccgacgtggtcacatacaacaccctgatcgacggcctgtgcaagagcggcaagatcgaggaggccctgaagctgttcaaggagatggaggagaagggcatcacccccagcgtggttacatacaccacactgatcgacggactgtgtaaagccggcgacgtggacgaagccctcgagctgttcaaagagatgcggagcaagggcgtgaagcccaacgtggtcacatacaccaccctgatcgacggcctgtgcaaggccggcaaggtggatgaggccctggagctgttcgacgagatgaaggagaggggcatcaagcccgacgtggtcacatacaacaccctgatcgacggcctgtgcaagagcggcaagatcgaggaggccctgaagctgttcaaggagatggaggagaagggcatcacccccagcgtggtcacatacaccacactgatcgacggactgtgtaaagccggcgacgtggacgaagccctcgagctgttcaaagagatgcggagcaagggcgtgaagcccaacgtggttacatacaccaccctgatcgacggcctgtgcaaggccggcaaagtggacgaggccctggagctgttcgacgagatgaaggagaggggcatcaagcccgacgtggtcacatacaacaccctgatcgacggcctgtgcaagagcggcaagatcgaggaggccctgaagctgttcaaggagatggaggagaagggcatcacccccagcgtggtgacatacaccacactgatcgacggactgtgtaaagccggcgacgtggacgaagccctcgagctgttcaaagagatgcggagcaagggcgtgaagcccaacgtggtcacatacaccaccctgatcgacggcctgtgcaaggccggcaaggtggatgaggccctggagctgttcgacgagatgaaggagaggggcatcaagcccgacgagctgacctacaacaccctgatcagcggcctgggcaaggccggcagagccagagacccccccgtgctcagtagccccaagaagaaacgcaaagtcgaggatccaaagaagaaaaggaaggttgaagaccccaagaaaaagaggaaggtgggttccgactataaggaccacgacggagactacaaggatcatgatattgattacaaagacgatgacgataagatggccccaaagaagaagcggaaggtcggacgctagmagvskgtgvvvdgdvnghksvrgggdatngktkcttgkvwtvttgygvacsrydhmkhdksamgyvrtskddgtyktravkgdtvnrkgdkdgnghkynnshyvytadkknckankrhnvdgsvadhyntgdgvdnhyshskskdnkrdhmvvtaagthgmdyksrhmgnsvvtyntndcksgkakkmkgtsvvtyttdgckagdvdakmrskgvknvvtyttdgckagkvdadmkrgkdvvtyntdgcksgkakkmkgtsvvtyttdgckagdvdakmrskgvknvvtyttdgckagkvdadmkrgkdvvtyntdgcksgkakkmkgtsvvtyttdgckagdvdakmrskgvknvvtyttdgckagkvdadmkrgkdvvtyntdgcksgkakkmkgtsvvtyttdgckagdvdakmrskgvknvvtyttdgckagkvdadmkrgkdvvtyntdgcksgkakkmkgtsvvtyttdgckagdvdakmrskgvknvvtyttdgckagkvdadmkrgkdvvtyntdgcksgkakkmkgtsvvtyttdgckagdvdakmrskgvknvvtyttdgckagkvdadmkrgkdtyntsggkagrardvsskkkrkvdkkkrkvdkkkrkvgsdykdhdgdykdhddykddddkmakkkrkvgr

SEQ ID NO: 27 - DNA, 3552 bp, synthetic construct
  misc_difference 1..3552: note = PPR vector
  misc_difference 2912: note = a, c, g, t or u
  misc_difference 2925: note = a, c, g, t or u
  misc_difference 2982: note = a, c, g, t or u
  misc_difference 2990: note = a, c, g, t or u
  misc_difference 2995..2996: note = a, c, g, t or u
  misc_difference 3007: note = a, c, g, t or u
  misc_difference 3011: note = a, c, g, t or u
  misc_difference 3015: note = a, c, g, t or u
  misc_difference 3025: note = a, c, g, t or u
  misc_difference 3032: note = a, c, g, t or u
  misc_difference 3042: note = a, c, g, t or u
  misc_difference 3066: note = a, c, g, t or u
  misc_difference 3072: note = a, c, g, t or u
  misc_difference 3074: note = a, c, g, t or u
  misc_difference 3114: note = a, c, g, t or u
  misc_difference 3142: note = a, c, g, t or u
  misc_difference 3183: note = a, c, g, t or u
  misc_difference 3211: note = a, c, g, t or u
  misc_difference 3252: note = a, c, g, t or u
  misc_difference 3280: note = a, c, g, t or u
  misc_difference 3321: note = a, c, g, t or u
  misc_difference 3349: note = a, c, g, t or u
  misc_difference 3390: note = a, c, g, t or u
  misc_difference 3418: note = a, c, g, t or u
  misc_difference 3459: note = a, c, g, t or u
  misc_difference 3485: note = a, c, g, t or u
  source 1..3552: mol_type = other DNA
atggccggagtgtccaaaggcgaggagctgtttaccggcgtcgtgcctattctggtggagctggacggcgacgtgaacggccacaagttctccgtgaggggcgagggcgaaggcgatgccacaaacggcaagctgaccctcaagttcatctgcaccactggtaaactgcccgttccttggcccacactggtgaccaccttcggctacggcgtggcttgtttctctcgttaccccgaccatatgaagcagcacgacttcttcaagtccgccatgcccgagggatacgtgcaagaaaggaccatctccttcaaggacgatggcacctacaagaccagagccgaggtgaagttcgagggcgacacactggtgaatcgtatcgaactgaagggcatcgacttcaaagaggacggcaacattctgggccacaagctggagtacaacttcaacagccactacgtgtacatcaccgccgataagcagaagaactgcatcaaggccaacttcaagattcgtcacaacgtggaggatggctccgtgcagctggccgatcactaccagcagaacacacccatcggcgatggacccgttttactgcccgacaaccactatttaagccaccagagcaagctgtccaaggaccccaacgagaagcgtgatcatatggtgctgctcgagtttgtgaccgccgccggcatcacccatggaatggacgagctgtacaagagccggctccatatgggaaactccgtggtcacatacaacaccaacatcgacaaactgtgcaagagcggcaagatcgaggaggccctgaagctgttcaaggagatggaggagaagggcatcacccccagcgtggtcacatacaccacactgatcgacggactgtgtaaagccggcgacgtggacgaagccctcgagctgttcaaagagatgcggagcaagggcgtgaagcccaacgtggtgacatacaccaccctgatcgacggcctgtgcaaggccggcaaagtggacgaggccctggagctgttcgacgagatgaaggagaggggcatcaagcccgacgtggtcacatacaacaccctgatcgacggcctgtgcaagagcggcaagatcgaggaggccctgaagctgttcaaggagatggaggagaagggcatcacccccagcgtggttacatacaccacactgatcgacggactgtgtaaagccggcgacgtggacgaagccctcgagctgttcaaagagatgcggagcaagggcgtgaagcccaacgtggtcacatacaccaccctgatcgacggcctgtgcaaggccggcaaggtggatgaggccctggagctgttcgacgagatgaaggagaggggcatcaagcccgacgtggttacatacaacaccctgatcgacggcctgtgcaagagcggcaagatcgaggaggccctgaagctgttcaaggagatggaggagaagggcatcacccccagcgtggtcacatacaccacactgatcgacggactgtgtaaagccggcgacgtggacgaagccctcgagctgttcaaagagatgcggagcaagggcgtgaagcccaacgtggtgacatacaccaccctgatcgacggcctgtgcaaggccggcaaagtggacgaggccctggagctgttcgacgagatgaaggagaggggcatcaagcccgacgtggtcacatacaacaccctgatcgacggcctgtgcaagagcggcaagatcgaggaggccctgaagctgttcaaggagatggaggagaagggcatcacccccagcgtggttacatacaccacactgatcgacggactgtgtaaagccggcgacgtggacgaagccctcgagctgttcaaagagatgcggagcaagggcgtgaagcccaacgtggtcacatacaccaccctgatcgacggcctgtgcaaggccggcaaggtggatgaggccctggagctgttcgacgagatgaaggagaggggcatcaagcccgacgtggtcacatacaacaccctgatcgacggcctgtgcaagagcggcaagatcgaggaggccctgaagctgttcaaggagatggaggagaagggcatcacccccagcgtggtcacatacaccacactgatcgacggactgtgtaaagccggcgacgtggacgaagccctcgagctgttcaaagagatgcggagcaagggcgtgaagcccaacgtggttacatacaccaccctgatcgacggcctgtgcaaggccggcaaagtggacgaggccctggagctgttcgacgagatgaaggagaggggcatcaagcccgacgtggtcacatacaacaccctgatcgacggcctgtgcaagagcggcaagatcgaggaggccctgaagctgttcaaggagatggaggagaagggcatcacccccagcgtggtgacatacaccacactgatcgacggactgtgtaaagccggcgacgtggacgaagccctcgagctgttcaaagagatgcggagcaagggcgtgaagcccaacgtggtcacatacaccaccctgatcgacggcctgtgcaaggccggcaaggtggatgaggccctggagctgttcgacgagatgaaggagaggggcatcaagcccgacgagctgacctacaacaccctgatcagcggcctgggcaaggccggcagagccagagacccccccgtgctcagtagccccaagaagaaacgcaaagtcgaggatccaaagaagaaaaggaaggttgaagaccccaagaaaaagaggaaggtgggttccgactataaggaccacgacggagactacaaggatcatgatattgattacaaagacgatgacgataagatggccccaaagaagaagcggaaggtcggacgctagmagvskgtgvvvdgdvnghksvrgggdatngktkcttgkvwtvttgygvacsrydhmkhdksamgyvrtskddgtyktravkgdtvnrkgdkdgnghkynnshyvytadkknckankrhnvdgsvadhyntgdgvdnhyshskskdnkrdhmvvtaagthgmdyksrhmgnsvvtyntndkcksgkakkmkgtsvvtyttdgckagdvdakmrskgvknvvtyttdgckagkvdadmkrgkdvvtyntdgcksgkakkmkgtsvvtyttdgckagdvdakmrskgvknvvtyttdgckagkvdadmkrgkdvvtyntdgcksgkakkmkgtsvvtyttdgckagdvdakmrskgvknvvtyttdgckagkvdadmkrgkdvvtyntdgcksgkakkmkgtsvvtyttdgckagdvdakmrskgvknvvtyttdgckagkvdadmkrgkdvvtyntdgcksgkakkmkgtsvvtyttdgckagdvdakmrskgvknvvtyttdgckagkvdadmkrgkdvvtyntdgcksgkakkmkgtsvvtyttdgckagdvdakmrskgvknvvtyttdgckagkvdadmkrgkdtyntsggkagrardvsskkkrkvdkkkrkvdkkkrkvgsdykdhdgdykdhddykddddkmakkkrkvgr

SEQ ID NO: 28 - DNA, 3552 bp, synthetic construct
  misc_difference 1..3552: note = PPR vector
  misc_difference 2912: note = a, c, g, t or u
  misc_difference 2925: note = a, c, g, t or u
  misc_difference 2982: note = a, c, g, t or u
  misc_difference 2990: note = a, c, g, t or u
  misc_difference 2995..2996: note = a, c, g, t or u
  misc_difference 3007: note = a, c, g, t or u
  misc_difference 3011: note = a, c, g, t or u
  misc_difference 3015: note = a, c, g, t or u
  misc_difference 3025: note = a, c, g, t or u
  misc_difference 3032: note = a, c, g, t or u
  misc_difference 3042: note = a, c, g, t or u
  misc_difference 3066: note = a, c, g, t or u
  misc_difference 3072: note = a, c, g, t or u
  misc_difference 3114: note = a, c, g, t or u
  misc_difference 3142: note = a, c, g, t or u
  misc_difference 3183: note = a, c, g, t or u
  misc_difference 3211: note = a, c, g, t or u
  misc_difference 3252: note = a, c, g, t or u
  misc_difference 3280: note = a, c, g, t or u
  misc_difference 3321: note = a, c, g, t or u
  misc_difference 3349: note = a, c, g, t or u
  misc_difference 3390: note = a, c, g, t or u
  misc_difference 3418: note = a, c, g, t or u
  misc_difference 3459: note = a, c, g, t or u
  misc_difference 3485: note = a, c, g, t or u
  source 1..3552: mol_type = other DNA
atggccggagtgtccaaaggcgaggagctgtttaccggcgtcgtgcctattctggtggagctggacggcgacgtgaacggccacaagttctccgtgaggggcgagggcgaaggcgatgccacaaacggcaagctgaccctcaagttcatctgcaccactggtaaactgcccgttccttggcccacactggtgaccaccttcggctacggcgtggcttgtttctctcgttaccccgaccatatgaagcagcacgacttcttcaagtccgccatgcccgagggatacgtgcaagaaaggaccatctccttcaaggacgatggcacctacaagaccagagccgaggtgaagttcgagggcgacacactggtgaatcgtatcgaactgaagggcatcgacttcaaagaggacggcaacattctgggccacaagctggagtacaacttcaacagccactacgtgtacatcaccgccgataagcagaagaactgcatcaaggccaacttcaagattcgtcacaacgtggaggatggctccgtgcagctggccgatcactaccagcagaacacacccatcggcgatggacccgttttactgcccgacaaccactatttaagccaccagagcaagctgtccaaggaccccaacgagaagcgtgatcatatggtgctgctcgagtttgtgaccgccgccggcatcacccatggaatggacgagctgtacaagagccggctccatatgggaaactccgtggtcacatacaacaccgatatcgacggcctgtgcaagagcggcaagatcgaggaggccctgaagctgttcaaggagatggaggagaagggcatcacccccagcgtggtcacatacaccacactgatcgacggactgtgtaaagccggcgacgtggacgaagccctcgagctgttcaaagagatgcggagcaagggcgtgaagcccaacgtggtgacatacaccaccctgatcgacggcctgtgcaaggccggcaaagtggacgaggccctggagctgttcgacgagatgaaggagaggggcatcaagcccgacgtggtcacatacaacaccctgatcgacggcctgtgcaagagcggcaagatcgaggaggccctgaagctgttcaaggagatggaggagaagggcatcacccccagcgtggttacatacaccacactgatcgacggactgtgtaaagccggcgacgtggacgaagccctcgagctgttcaaagagatgcggagcaagggcgtgaagcccaacgtggtcacatacaccaccctgatcgacggcctgtgcaaggccggcaaggtggatgaggccctggagctgttcgacgagatgaaggagaggggcatcaagcccgacgtggttacatacaacaccctgatcgacggcctgtgcaagagcggcaagatcgaggaggccctgaagctgttcaaggagatggaggagaagggcatcacccccagcgtggtcacatacaccacactgatcgacggactgtgtaaagccggcgacgtggacgaagccctcgagctgttcaaagagatgcggagcaagggcgtgaagcccaacgtggtgacatacaccaccctgatcgacggcctgtgcaaggccggcaaagtggacgaggccctggagctgttcgacgagatgaaggagaggggcatcaagcccgacgtggtcacatacaacaccctgatcgacggcctgtgcaagagcggcaagatcgaggaggccctgaagctgttcaaggagatggaggagaagggcatcacccccagcgtggttacatacaccacactgatcgacggactgtgtaaagccggcgacgtggacgaagccctcgagctgttcaaagagatgcggagcaagggcgtgaagcccaacgtggtcacatacaccaccctgatcgacggcctgtgcaaggccggcaaggtggatgaggccctggagctgttcgacgagatgaaggagaggggcatcaagcccgacgtggtcacatacaacaccctgatcgacggcctgtgcaagagcggcaagatcgaggaggccctgaagctgttcaaggagatggaggagaagggcatcacccccagcgtggtcacatacaccacactgatcgacggactgtgtaaagccggcgacgtggacgaagccctcgagctgttcaaagagatgcggagcaagggcgtgaagcccaacgtggttacatacaccaccctgatcgacggcctgtgcaaggccggcaaagtggacgaggccctggagctgttcgacgagatgaaggagaggggcatcaagcccgacgtggtcacatacaacaccctgatcgacggcctgtgcaagagcggcaagatcgaggaggccctgaagctgttcaaggagatggaggagaagggcatcacccccagcgtggtgacatacaccacactgatcgacggactgtgtaaagccggcgacgtggacgaagccctcgagctgttcaaagagatgcggagcaagggcgtgaagcccaacgtggtcacatacaccaccctgatcgacggcctgtgcaaggccggcaaggtggatgaggccctggagctgttcgacgagatgaaggagaggggcatcaagcccgacgagctgacctacaacaccctgatcagcggcctgggcaaggccggcagagccagagacccccccgtgctcagtagccccaagaagaaacgcaaagtcgaggatccaaagaagaaaaggaaggttgaagaccccaagaaaaagaggaaggtgggttccgactataaggaccacgacggagactacaaggatcatgatattgattacaaagacgatgacgataagatggccccaaagaagaagcggaaggtcggacgctagmagvskgtgvvvdgdvnghksvrgggdatngktkcttgkvwtvttgygvacsrydhmkhdksamgyvrtskddgtyktravkgdtvnrkgdkdgnghkynnshyvytadkknckankrhnvdgsvadhyntgdgvdnhyshskskdnkrdhmvvtaagthgmdyksrhmgnsvvtyntddgcksgkakkmkgtsvvtyttdgckagdvdakmrskgvknvvtyttdgckagkvdadmkrgkdvvtyntdgcksgkakkmkgtsvvtyttdgckagdvdakmrskgvknvvtyttdgckagkvdadmkrgkdvvtyntdgcksgkakkmkgtsvvtyttdgckagdvdakmrskgvknvvtyttdgckagkvdadmkrgkdvvtyntdgcksgkakkmkgtsvvtyttdgckagdvdakmrskgvknvvtyttdgckagkvdadmkrgkdvvtyntdgcksgkakkmkgtsvvtyttdgckagdvdakmrskgvknvvtyttdgckagkvdadmkrgkdvvtyntdgcksgkakkmkgtsvvtyttdgckagdvdakmrskgvknvvtyttdgckagkvdadmkrgkdtyntsggkagrardvsskkkrkvdkkkrkvdkkkrkvgsdykdhdgdykdhddykddddkmakkkrkvgr

SEQ ID NO: 29 - DNA, 3189 bp, synthetic construct
  misc_difference 1..3189: note = NL_PPRcag_1
  misc_difference 2626: note = a, c, g, t or u
  misc_difference 2634: note = a, c, g, t or u
  misc_difference 2644: note = a, c, g, t or u
  misc_difference 2678: note = a, c, g, t or u
  misc_difference 2698: note = a, c, g, t or u
  misc_difference 2700: note = a, c, g, t or u
  misc_difference 2704: note = a, c, g, t or u
  misc_difference 2711: note = a, c, g, t or u
  misc_difference 2740: note = a, c, g, t or u
  misc_difference 2746: note = a, c, g, t or u
  misc_difference 2787: note = a, c, g, t or u
  misc_difference 2815: note = a, c, g, t or u
  misc_difference 2856: note = a, c, g, t or u
  misc_difference 2884: note = a, c, g, t or u
  misc_difference 2925: note = a, c, g, t or u
  misc_difference 2953: note = a, c, g, t or u
  misc_difference 2994: note = a, c, g, t or u
  misc_difference 3022: note = a, c, g, t or u
  misc_difference 3063: note = a, c, g, t or u
  misc_difference 3091: note = a, c, g, t or u
  misc_difference 3132: note = a, c, g, t or u
  misc_difference 3158: note = a, c, g, t or u
  source 1..3189: mol_type = other DNA
atggccggagtgttcacactggaggacttcgtgggcgactggagacagaccgccggctacaacctggaccaggtgctggagcagggcggagtgagcagcctgtttcagaacctgggcgtgagcgtcacccccatccagaggatcgtgctgtccggcgagaatggcctgaagatcgacatccacgtcatcatcccctacgagggcctgagcggcgatcagatgggccagatcgagaagatcttcaaggtggtgtatcccgtcgacgaccaccacttcaaggtgatcctgcattacggcaccctcgtgatcgacggcgtgacccctaacatgatcgactacttcggcaggccctacgagggaatcgccgtgttcgacggaaagaagatcaccgtgaccggcaccctgtggaacggaaacaagatcatcgacgagaggctgatcaaccccgacggctccctgctgttcagggtgaccatcaatggcgtgaccggctggagactgtgcgagagaatcctggccggaggcggaggaagcctcgtgcccagaggatccggcggaggcggctccagccggctccatatgggaaactccgtggtcacatacaacaccctgatcgacggcctgtgcaagagcggcaagatcgaggaggccctgaagctgttcaaggagatggaggagaagggcatcacccccagcgtggtcacatacaccacactgatcgacggactgtgtaaagccggcgacgtggacgaagccctcgagctgttcaaagagatgcggagcaagggcgtgaagcccaacgtggtgacatacaccaccctgatcgacggcctgtgcaaggccggcaaagtggacgaggccctggagctgttcgacgagatgaaggagaggggcatcaagcccgacgtggtcacatacaacaccctgatcgacggcctgtgcaagagcggcaagatcgaggaggccctgaagctgttcaaggagatggaggagaagggcatcacccccagcgtggttacatacaccacactgatcgacggactgtgtaaagccggcgacgtggacgaagccctcgagctgttcaaagagatgcggagcaagggcgtgaagcccaacgtggtcacatacaccaccctgatcgacggcctgtgcaaggccggcaaggtggatgaggccctggagctgttcgacgagatgaaggagaggggcatcaagcccgacgtggttacatacaacaccctgatcgacggcctgtgcaagagcggcaagatcgaggaggccctgaagctgttcaaggagatggaggagaagggcatcacccccagcgtggtcacatacaccacactgatcgacggactgtgtaaagccggcgacgtggacgaagccctcgagctgttcaaagagatgcggagcaagggcgtgaagcccaacgtggtgacatacaccaccctgatcgacggcctgtgcaaggccggcaaagtggacgaggccctggagctgttcgacgagatgaaggagaggggcatcaagcccgacgtggtcacatacaacaccctgatcgacggcctgtgcaagagcggcaagatcgaggaggccctgaagctgttcaaggagatggaggagaagggcatcacccccagcgtggttacatacaccacactgatcgacggactgtgtaaagccggcgacgtggacgaagccctcgagctgttcaaagagatgcggagcaagggcgtgaagcccaacgtggtcacatacaccaccctgatcgacggcctgtgcaaggccggcaaggtggatgaggccctggagctgttcgacgagatgaaggagaggggcatcaagcccgacgtggtcacatacaacaccctgatcgacggcctgtgcaagagcggcaagatcgaggaggccctgaagctgttcaaggagatggaggagaagggcatcacccccagcgtggtcacatacaccacactgatcgacggactgtgtaaagccggcgacgtggacgaagccctcgagctgttcaaagagatgcggagcaagggcgtgaagcccaacgtggttacatacaccaccctgatcgacggcctgtgcaaggccggcaaagtggacgaggccctggagctgttcgacgagatgaaggagaggggcatcaagcccgacgtggtcacatacaacaccctgatcgacggcctgtgcaagagcggcaagatcgaggaggccctgaagctgttcaaggagatggaggagaagggcatcacccccagcgtggtgacatacaccacactgatcgacggactgtgtaaagccggcgacgtggacgaagccctcgagctgttcaaagagatgcggagcaagggcgtgaagcccaacgtggtcacatacaccaccctgatcgacggcctgtgcaaggccggcaaggtggatgaggccctggagctgttcgacgagatgaaggagaggggcatcaagcccgacgagctgacctacaacaccctgatcagcggcctgggcaaggccggcagagccagagacccccccgtgctcagtagcggctccggcggcagcgggggaggcgggcatcatcaccatcaccacggacgctagmagvtdvgdwrtagyndvggvssngvsvtrvsgngkdhvygsgdmgkkvvyvddhhkvhygtvdgvtnmdygrygavdgkktvtgtwngnkdrndgsrvtngvtgwrcraggggsvrgsggggssrhmgnsvvtyntdgcksgkakkmkgtsvvtyttdgckagdvdakmrskgvknvvtyttdgckagkvdadmkrgkdvvtyntdgcksgkakkmkgtsvvtyttdgckagdvdakmrskgvknvvtyttdgckagkvdadmkrgkdvvtyntdgcksgkakkmkgtsvvtyttdgckagdvdakmrskgvknvvtyttdgckagkvdadmkrgkdvvtyntdgcksgkakkmkgtsvvtyttdgckagdvdakmrskgvknvvtyttdgckagkvdadmkrgkdvvtyntdgcksgkakkmkgtsvvtyttdgckagdvdakmrskgvknvvtyttdgckagkvdadmkrgkdvvtyntdgcksgkakkmkgtsvvtyttdgckagdvdakmrskgvknvvtyttdgckagkvdadmkrgkdtyntsggkagrardvssgsggsgggghhhhhhgr

SEQ ID NO: 30 - DNA, 3188 bp, synthetic construct
  misc_difference 1..3188: note = NL_PPRcag_2
  misc_difference 2626: note = a, c, g, t or u
  misc_difference 2634: note = a, c, g, t or u
  misc_difference 2644: note = a, c, g, t or u
  misc_difference 2678: note = a, c, g, t or u
  misc_difference 2698: note = a, c, g, t or u
  misc_difference 2700: note = a, c, g, t or u
  misc_difference 2704: note = a, c, g, t or u
  misc_difference 2711: note = a, c, g, t or u
  misc_difference 2740: note = a, c, g, t or u
  misc_difference 2746: note = a, c, g, t or u
  misc_difference 2786: note = a, c, g, t or u
  misc_difference 2814: note = a, c, g, t or u
  misc_difference 2855: note = a, c, g, t or u
  misc_difference 2883: note = a, c, g, t or u
  misc_difference 2924: note = a, c, g, t or u
  misc_difference 2952: note = a, c, g, t or u
  misc_difference 2993: note = a, c, g, t or u
  misc_difference 3021: note = a, c, g, t or u
  misc_difference 3062: note = a, c, g, t or u
  misc_difference 3090: note = a, c, g, t or u
  misc_difference 3131: note = a, c, g, t or u
  misc_difference 3157: note = a, c, g, t or u
  source 1..3188: mol_type = other DNA
atggccggagtgttcacactggaggacttcgtgggcgactggagacagaccgccggctacaacctggaccaggtgctggagcagggcggagtgagcagcctgtttcagaacctgggcgtgagcgtcacccccatccagaggatcgtgctgtccggcgagaatggcctgaagatcgacatccacgtcatcatcccctacgagggcctgagcggcgatcagatgggccagatcgagaagatcttcaaggtggtgtatcccgtcgacgaccaccacttcaaggtgatcctgcattacggcaccctcgtgatcgacggcgtgacccctaacatgatcgactacttcggcaggccctacgagggaatcgccgtgttcgacggaaagaagatcaccgtgaccggcaccctgtggaacggaaacaagatcatcgacgagaggctgatcaaccccgacggctccctgctgttcagggtgaccatcaatggcgtgaccggctggagactgtgcgagagaatcctggccggaggcggaggaagcctcgtgcccagaggatccggcggaggcggctccagccggctccatatgggaaactccgtggtcacatacaacaccctgatcgacgaactgtgcaagagcggcaagatcgaggaggccctgaagctgttcaaggagatggaggagaagggcatcacccccagcgtggtcacatacaccacactgatcgacggactgtgtaaagccggcgacgtggacgaagccctcgagctgttcaaagagatgcggagcaagggcgtgaagcccaacgtggtgacatacaccaccctgatcgacggcctgtgcaaggccggcaaagtggacgaggccctggagctgttcgacgagatgaaggagaggggcatcaagcccgacgtggtcacatacaacaccctgatcgacggcctgtgcaagagcggcaagatcgaggaggccctgaagctgttcaaggagatggaggagaagggcatcacccccagcgtggttacatacaccacactgatcgacggactgtgtaaagccggcgacgtggacgaagccctcgagctgttcaaagagatgcggagcaagggcgtgaagcccaacgtggtcacatacaccaccctgatcgacggcctgtgcaaggccggcaaggtggatgaggccctggagctgttcgacgagatgaaggagaggggcatcaagcccgacgtggttacatacaacaccctgatcgacggcctgtgcaagagcggcaagatcgaggaggccctgaagctgttcaaggagatggaggagaagggcatcacccccagcgtggtcacatacaccacactgatcgacggactgtgtaaagccggcgacgtggacgaagccctcgagctgttcaaagagatgcggagcaagggcgtgaagcccaacgtggtgacatacaccaccctgatcgacggcctgtgcaaggccggcaaagtggacgaggccctggagctgttcgacgagatgaaggagaggggcatcaagcccgacgtggtcacatacaacaccctgatcgacggcctgtgcaagagcggcaagatcgaggaggccctgaagctgttcaaggagatggaggagaagggcatcacccccagcgtggttacatacaccacactgatcgacggactgtgtaaagccggcgacgtggacgaagccctcgagctgttcaaagagatgcggagcaagggcgtgaagcccaacgtggtcacatacaccaccctgatcgacggcctgtgcaaggccggcaaggtggatgaggccctggagctgttcgacgagatgaaggagaggggcatcaagcccgacgtggtcacatacaacaccctgatcgacggcctgtgcaagagcggcaagatcgaggaggccctgaagctgttcaaggagatggaggagaagggcatcacccccagcgtggtcacatacaccacactgatcgacggactgtgtaaagccggcgacgtggacgaagccctcgagctgttcaaagagatgcggagcaagggcgtgaagcccaacgtggttacatacaccaccctgatcgacggcctgtgcaaggccggcaaagtggacgaggccctggagctgttcgacgagatgaaggagaggggcatcaagcccgacgtggtcacatacaacaccctgatcgacggcctgtgcaagagcggcaagatcgaggaggccctgaagctgttcaaggagatggaggagaagggcatcacccccagcgtggtgacatacaccacactgatcgacggactgtgtaaagccggcgacgtggacgaagccctcgagctgttcaaagagatgcggagcaagggcgtgaagcccaacgtggtcacatacaccaccctgatcgacggcctgtgcaaggccggcaaggtggatgaggccctggagctgttcgacgagatgaaggagaggggcatcaagcccgacgagctgacctacaacaccctgatcagcggcctgggcaaggccggcagagccagagacccccccgtgctcagtagcggctccggcggcagcgggggaggcgggcatcatcaccatcaccacggacgctagmagvtdvgdwrtagyndvggvssngvsvtrvsgngkdhvygsgdmgkkvvyvddhhkvhygtvdgvtnmdygrygavdgkktvtgtwngnkdrndgsrvtngvtgwrcraggggsvrgsggggssrhmgnsvvtyntdcksgkakkmkgtsvvtyttdgckagdvdakmrskgvknvvtyttdgckagkvdadmkrgkdvvtyntdgcksgkakkmkgtsvvtyttdgckagdvdakmrskgvknvvtyttdgckagkvdadmkrgkdvvtyntdgcksgkakkmkgtsvvtyttdgckagdvdakmrskgvknvvtyttdgckagkvdadmkrgkdvvtyntdgcksgkakkmkgtsvvtyttdgckagdvdakmrskgvknvvtyttdgckagkvdadmkrgkdvvtyntdgcksgkakkmkgtsvvtyttdgckagdvdakmrskgvknvvtyttdgckagkvdadmkrgkdvvtyntdgcksgkakkmkgtsvvtyttdgckagdvdakmrskgvknvvtyttdgckagkvdadmkrgkdtyntsggkagrardvssgsggsgggghhhhhhgr

SEQ ID NO: 31 - DNA, 3189 bp, synthetic construct
  misc_difference 1..3189: note = NL_PPRcag_3
  misc_difference 2626: note = a, c, g, t or u
  misc_difference 2634: note = a, c, g, t or u
  misc_difference 2644: note = a, c, g, t or u
  misc_difference 2678: note = a, c, g, t or u
  misc_difference 2698: note = a, c, g, t or u
  misc_difference 2700: note = a, c, g, t or u
  misc_difference 2704: note = a, c, g, t or u
  misc_difference 2711: note = a, c, g, t or u
  misc_difference 2740: note = a, c, g, t or u
  misc_difference 2746: note = a, c, g, t or u
  misc_difference 2748: note = a, c, g, t or u
  misc_difference 2787: note = a, c, g, t or u
  misc_difference 2815: note = a, c, g, t or u
  misc_difference 2856: note = a, c, g, t or u
  misc_difference 2884: note = a, c, g, t or u
  misc_difference 2925: note = a, c, g, t or u
  misc_difference 2953: note = a, c, g, t or u
  misc_difference 2994: note = a, c, g, t or u
  misc_difference 3022: note = a, c, g, t or u
  misc_difference 3063: note = a, c, g, t or u
  misc_difference 3091: note = a, c, g, t or u
  misc_difference 3132: note = a, c, g, t or u
  misc_difference 3158: note = a, c, g, t or u
  source 1..3189: mol_type = other DNA
atggccggagtgttcacactggaggacttcgtgggcgactggagacagaccgccggctacaacctggaccaggtgctggagcagggcggagtgagcagcctgtttcagaacctgggcgtgagcgtcacccccatccagaggatcgtgctgtccggcgagaatggcctgaagatcgacatccacgtcatcatcccctacgagggcctgagcggcgatcagatgggccagatcgagaagatcttcaaggtggtgtatcccgtcgacgaccaccacttcaaggtgatcctgcattacggcaccctcgtgatcgacggcgtgacccctaacatgatcgactacttcggcaggccctacgagggaatcgccgtgttcgacggaaagaagatcaccgtgaccggcaccctgtggaacggaaacaagatcatcgacgagaggctgatcaaccccgacggctccctgctgttcagggtgaccatcaatggcgtgaccggctggagactgtgcgagagaatcctggccggaggcggaggaagcctcgtgcccagaggatccggcggaggcggctccagccggctccatatgggaaactccgtggtcacatacaacaccaacatcgaccagctgtgcaagagcggcaagatcgaggaggccctgaagctgttcaaggagatggaggagaagggcatcacccccagcgtggtcacatacaccacactgatcgacggactgtgtaaagccggcgacgtggacgaagccctcgagctgttcaaagagatgcggagcaagggcgtgaagcccaacgtggtgacatacaccaccctgatcgacggcctgtgcaaggccggcaaagtggacgaggccctggagctgttcgacgagatgaaggagaggggcatcaagcccgacgtggtcacatacaacaccctgatcgacggcctgtgcaagagcggcaagatcgaggaggccctgaagctgttcaaggagatggaggagaagggcatcacccccagcgtggttacatacaccacactgatcgacggactgtgtaaagccggcgacgtggacgaagccctcgagctgttcaaagagatgcggagcaagggcgtgaagcccaacgtggtcacatacaccaccctgatcgacggcctgtgcaaggccggcaaggtggatgaggccctggagctgttcgacgagatgaaggagaggggcatcaagcccgacgtggttacatacaacaccctgatcgacggcctgtgcaagagcggcaagatcgaggaggccctgaagctgttcaaggagatggaggagaagggcatcacccccagcgtggtcacatacaccacactgatcgacggactgtgtaaagccggcgacgtggacgaagccctcgagctgttcaaagagatgcggagcaagggcgtgaagcccaacgtggtgacatacaccaccctgatcgacggcctgtgcaaggccggcaaagtggacgaggccctggagctgttcgacgagatgaaggagaggggcatcaagcccgacgtggtcacatacaacaccctgatcgacggcctgtgcaagagcggcaagatcgaggaggccctgaagctgttcaaggagatggaggagaagggcatcacccccagcgtggttacatacaccacactgatcgacggactgtgtaaagccggcgacgtggacgaagccctcgagctgttcaaagagatgcggagcaagggcgtgaagcccaacgtggtcacatacaccaccctgatcgacggcctgtgcaaggccggcaaggtggatgaggccctggagctgttcgacgagatgaaggagaggggcatcaagcccgacgtggtcacatacaacaccctgatcgacggcctgtgcaagagcggcaagatcgaggaggccctgaagctgttcaaggagatggaggagaagggcatcacccccagcgtggtcacatacaccacactgatcgacggactgtgtaaagccggcgacgtggacgaagccctcgagctgttcaaagagatgcggagcaagggcgtgaagcccaacgtggttacatacaccaccctgatcgacggcctgtgcaaggccggcaaagtggacgaggccctggagctgttcgacgagatgaaggagaggggcatcaagcccgacgtggtcacatacaacaccctgatcgacggcctgtgcaagagcggcaagatcgaggaggccctgaagctgttcaaggagatggaggagaagggcatcacccccagcgtggtgacatacaccacactgatcgacggactgtgtaaagccggcgacgtggacgaagccctcgagctgttcaaagagatgcggagcaagggcgtgaagcccaacgtggtcacatacaccaccctgatcgacggcctgtgcaaggccggcaaggtggatgaggccctggagctgttcgacgagatgaaggagaggggcatcaagcccgacgagctgacctacaacaccctgatcagcggcctgggcaaggccggcagagccagagacccccccgtgctcagtagcggctccggcggcagcgggggaggcgggcatcatcaccatcaccacggacgctagmagvtdvgdwrtagyndvggvssngvsvtrvsgngkdhvygsgdmgkkvvyvddhhkvhygtvdgvtnmdygrygavdgkktvtgtwngnkdrndgsrvtngvtgwrcraggggsvrgsggggssrhmgnsvvtyntndcksgkakkmkgtsvvtyttdgckagdvdakmrskgvknvvtyttdgckagkvdadmkrgkdvvtyntdgcksgkakkmkgtsvvtyttdgckagdvdakmrskgvknvvtyttdgckagkvdadmkrgkdvvtyntdgcksgkakkmkgtsvvtyttdgckagdvdakmrskgvknvvtyttdgckagkvdadmkrgkdvvtyntdgcksgkakkmkgtsvvtyttdgckagdvdakmrskgvknvvtyttdgckagkvdadmkrgkdvvtyntdgcksgkakkmkgtsvvtyttdgckagdvdakmrskgvknvvtyttdgckagkvdadmkrgkdvvtyntdgcksgkakkmkgtsvvtyttdgckagdvdakmrskgvknvvtyttdgckagkvdadmkrgkdtyntsggkagrardvssgsggsgggghhhhhhgr

SEQ ID NO: 32 - DNA, 3189 bp, synthetic construct
  misc_difference 1..3189: note = NL_PPRcag_4
  misc_difference 2626: note = a, c, g, t or u
  misc_difference 2634: note = a, c, g, t or u
  misc_difference 2644: note = a, c, g, t or u
  misc_difference 2678: note = a, c, g, t or u
  misc_difference 2698: note = a, c, g, t or u
  misc_difference 2700: note = a, c, g, t or u
  misc_difference 2704: note = a, c, g, t or u
  misc_difference 2711: note = a, c, g, t or u
  misc_difference 2740: note = a, c, g, t or u
  misc_difference 2746: note = a, c, g, t or u
  misc_difference 2748: note = a, c, g, t or u
  misc_difference 2787: note = a, c, g, t or u
  misc_difference 2815: note = a, c, g, t or u
  misc_difference 2856: note = a, c, g, t or u
  misc_difference 2884: note = a, c, g, t or u
  misc_difference 2925: note = a, c, g, t or u
  misc_difference 2953: note = a, c, g, t or u
  misc_difference 2994: note = a, c, g, t or u
  misc_difference 3022: note = a, c, g, t or u
  misc_difference 3063: note = a, c, g, t or u
  misc_difference 3091: note = a, c, g, t or u
  misc_difference 3132: note = a, c, g, t or u
  misc_difference 3158: note = a, c, g, t or u
  source 1..3189: mol_type = other DNA
atggccggagtgttcacactggaggacttcgtgggcgactggagacagaccgccggctacaacctggaccaggtgctggagcagggcggagtgagcagcctgtttcagaacctgggcgtgagcgtcacccccatccagaggatcgtgctgtccggcgagaatggcctgaagatcgacatccacgtcatcatcccctacgagggcctgagcggcgatcagatgggccagatcgagaagatcttcaaggtggtgtatcccgtcgacgaccaccacttcaaggtgatcctgcattacggcaccctcgtgatcgacggcgtgacccctaacatgatcgactacttcggcaggccctacgagggaatcgccgtgttcgacggaaagaagatcaccgtgaccggcaccctgtggaacggaaacaagatcatcgacgagaggctgatcaaccccgacggctccctgctgttcagggtgaccatcaatggcgtgaccggctggagactgtgcgagagaatcctggccggaggcggaggaagcctcgtgcccagaggatccggcggaggcggctccagccggctccatatgggaaactccgtggtcacatacaacaccaacatcgacgaactgtgcaagagcggcaagatcgaggaggccctgaagctgttcaaggagatggaggagaagggcatcacccccagcgtggtcacatacaccacactgatcgacggactgtgtaaagccggcgacgtggacgaagccctcgagctgttcaaagagatgcggagcaagggcgtgaagcccaacgtggtgacatacaccaccctgatcgacggcctgtgcaaggccggcaaagtggacgaggccctggagctgttcgacgagatgaaggagaggggcatcaagcccgacgtggtcacatacaacaccctgatcgacggcctgtgcaagagcggcaagatcgaggaggccctgaagctgttcaaggagatggaggagaagggcatcacccccagcgtggttacatacaccacactgatcgacggactgtgtaaagccggcgacgtggacgaagccctcgagctgttcaaagagatgcggagcaagggcgtgaagcccaacgtggtcacatacaccaccctgatcgacggcctgtgcaaggccggcaaggtggatgaggccctggagctgttcgacgagatgaaggagaggggcatcaagcccgacgtggttacatacaacaccctgatcgacggcctgtgcaagagcggcaagatcgaggaggccctgaagctgttcaaggagatggaggagaagggcatcacccccagcgtggtcacatacaccacactgatcgacggactgtgtaaagccggcgacgtggacgaagccctcgagctgttcaaagagatgcggagcaagggcgtgaagcccaacgtggtgacatacaccaccctgatcgacggcctgtgcaaggccggcaaagtggacgaggccctggagctgttcgacgagatgaaggagaggggcatcaagcccgacgtggtcacatacaacaccctgatcgacggcctgtgcaagagcggcaagatcgaggaggccctgaagctgttcaaggagatggaggagaagggcatcacccccagcgtggttacatacaccacactgatcgacggactgtgtaaagccggcgacgtggacgaagccctcgagctgttcaaagagatgcggagcaagggcgtgaagcccaacgtggtcacatacaccaccctgatcgacggcctgtgcaaggccggcaaggtggatgaggccctggagctgttcgacgagatgaaggagaggggcatcaagcccgacgtggtcacatacaacaccctgatcgacggcctgtgcaagagcggcaagatcgaggaggccctgaagctgttcaaggagatggaggagaagggcatcacccccagcgtggtcacatacaccacactgatcgacggactgtgtaaagccggcgacgtggacgaagccctcgagctgttcaaagagatgcggagcaagggcgtgaagcccaacgtggttacatacaccaccctgatcgacggcctgtgcaaggccggcaaagtggacgaggccctggagctgttcgacgagatgaaggagaggggcatcaagcccgacgtggtcacatacaacaccctgatcgacggcctgtgcaagagcggcaagatcgaggaggccctgaagctgttcaaggagatggaggagaagggcatcacccccagcgtggtgacatacaccacactgatcgacggactgtgtaaagccggcgacgtggacgaagccctcgagctgttcaaagagatgcggagcaagggcgtgaagcccaacgtggtcacatacaccaccctgatcgacggcctgtgcaaggccggcaaggtggatgaggccctggagctgttcgacgagatgaaggagaggggcatcaagcccgacgagctgacctacaacaccctgatcagcggcctgggcaaggccggcagagccagagacccccccgtgctcagtagcggctccggcggcagcgggggaggcgggcatcatcaccatcaccacggacgctagmagvtdvgdwrtagyndvggvssngvsvtrvsgngkdhvygsgdmgkkvvyvddhhkvhygtvdgvtnmdygrygavdgkktvtgtwngnkdrndgsrvtngvtgwrcraggggsvrgsggggssrhmgnsvvtyntndcksgkakkmkgtsvvtyttdgckagdvdakmrskgvknvvtyttdgckagkvdadmkrgkdvvtyntdgcksgkakkmkgtsvvtyttdgckagdvdakmrskgvknvvtyttdgckagkvdadmkrgkdvvtyntdgcksgkakkmkgtsvvtyttdgckagdvdakmrskgvknvvtyttdgckagkvdadmkrgkdvvtyntdgcksgkakkmkgtsvvtyttdgckagdvdakmrskgvknvvtyttdgckagkvdadmkrgkdvvtyntdgcksgkakkmkgtsvvtyttdgckagdvdakmrskgvknvvtyttdgckagkvdadmkrgkdvvtyntdgcksgkakkmkgtsvvtyttdgckagdvdakmrskgvknvvtyttdgckagkvdadmkrgkdtyntsggkagrardvssgsggsgggghhhhhhgr

SEQ ID NO: 33 - DNA, 3190 bp, synthetic construct
  misc_difference 1..3190: note = NL_PPRcag_5
  misc_difference 2626: note = a, c, g, t or u
  misc_difference 2634: note = a, c, g, t or u
  misc_difference 2644: note = a, c, g, t or u
  misc_difference 2678: note = a, c, g, t or u
  misc_difference 2698: note = a, c, g, t or u
  misc_difference 2700: note = a, c, g, t or u
  misc_difference 2704: note = a, c, g, t or u
  misc_difference 2711: note = a, c, g, t or u
  misc_difference 2740: note = a, c, g, t or u
  misc_difference 2746: note = a, c, g, t or u
  misc_difference 2748: note = a, c, g, t or u
  misc_difference 2788: note = a, c, g, t or u
  misc_difference 2816: note = a, c, g, t or u
  misc_difference 2857: note = a, c, g, t or u
  misc_difference 2885: note = a, c, g, t or u
  misc_difference 2926: note = a, c, g, t or u
  misc_difference 2954: note = a, c, g, t or u
  misc_difference 2995: note = a, c, g, t or u
  misc_difference 3023: note = a, c, g, t or u
  misc_difference 3064: note = a, c, g, t or u
  misc_difference 3092: note = a, c, g, t or u
  misc_difference 3133: note = a, c, g, t or u
  misc_difference 3159: note = a, c, g, t or u
  source 1..3190: mol_type = other DNA
atggccggagtgttcacactggaggacttcgtgggcgactggagacagaccgccggctacaacctggaccaggtgctggagcagggcggagtgagcagcctgtttcagaacctgggcgtgagcgtcacccccatccagaggatcgtgctgtccggcgagaatggcctgaagatcgacatccacgtcatcatcccctacgagggcctgagcggcgatcagatgggccagatcgagaagatcttcaaggtggtgtatcccgtcgacgaccaccacttcaaggtgatcctgcattacggcaccctcgtgatcgacggcgtgacccctaacatgatcgactacttcggcaggccctacgagggaatcgccgtgttcgacggaaagaagatcaccgtgaccggcaccctgtggaacggaaacaagatcatcgacgagaggctgatcaaccccgacggctccctgctgttcagggtgaccatcaatggcgtgaccggctggagactgtgcgagagaatcctggccggaggcggaggaagcctcgtgcccagaggatccggcggaggcggctccagccggctccatatgggaaactccgtggtcacatacaacaccaacatcgacaaactgtgcaagagcggcaagatcgaggaggccctgaagctgttcaaggagatggaggagaagggcatcacccccagcgtggtcacatacaccacactgatcgacggactgtgtaaagccggcgacgtggacgaagccctcgagctgttcaaagagatgcggagcaagggcgtgaagcccaacgtggtgacatacaccaccctgatcgacggcctgtgcaaggccggcaaagtggacgaggccctggagctgttcgacgagatgaaggagaggggcatcaagcccgacgtggtcacatacaacaccctgatcgacggcctgtgcaagagcggcaagatcgaggaggccctgaagctgttcaaggagatggaggagaagggcatcacccccagcgtggttacatacaccacactgatcgacggactgtgtaaagccggcgacgtggacgaagccctcgagctgttcaaagagatgcggagcaagggcgtgaagcccaacgtggtcacatacaccaccctgatcgacggcctgtgcaaggccggcaaggtggatgaggccctggagctgttcgacgagatgaaggagaggggcatcaagcccgacgtggttacatacaacaccctgatcgacggcctgtgcaagagcggcaagatcgaggaggccctgaagctgttcaaggagatggaggagaagggcatcacccccagcgtggtcacatacaccacactgatcgacggactgtgtaaagccggcgacgtggacgaagccctcgagctgttcaaagagatgcggagcaagggcgtgaagcccaacgtggtgacatacaccaccctgatcgacggcctgtgcaaggccggcaaagtggacgaggccctggagctgttcgacgagatgaaggagaggggcatcaagcccgacgtggtcacatacaacaccctgatcgacggcctgtgcaagagcggcaagatcgaggaggccctgaagctgttcaaggagatggaggagaagggcatcacccccagcgtggttacatacaccacactgatcgacggactgtgtaaagccggcgacgtggacgaagccctcgagctgttcaaagagatgcggagcaagggcgtgaagcccaacgtggtcacatacaccaccctgatcgacggcctgtgcaaggccggcaaggtggatgaggccctggagctgttcgacgagatgaaggagaggggcatcaagcccgacgtggtcacatacaacaccctgatcgacggcctgtgcaagagcggcaagatcgaggaggccctgaagctgttcaaggagatggaggagaagggcatcacccccagcgtggtcacatacaccacactgatcgacggactgtgtaaagccggcgacgtggacgaagccctcgagctgttcaaagagatgcggagcaagggcgtgaagcccaacgtggttacatacaccaccctgatcgacggcctgtgcaaggccggcaaagtggacgaggccctggagctgttcgacgagatgaaggagaggggcatcaagcccgacgtggtcacatacaacaccctgatcgacggcctgtgcaagagcggcaagatcgaggaggccctgaagctgttcaaggagatggaggagaagggcatcacccccagcgtggtgacatacaccacactgatcgacggactgtgtaaagccggcgacgtggacgaagccctcgagctgttcaaagagatgcggagcaagggcgtgaagcccaacgtggtcacatacaccaccctgatcgacggcctgtgcaaggccggcaaggtggatgaggccctggagctgttcgacgagatgaaggagaggggcatcaagcccgacgagctgacctacaacaccctgatcagcggcctgggcaaggccggcagagccagagacccccccgtgctcagtagcggctccggcggcagcgggggaggcgggcatcatcaccatcaccacggacgctagmagvtdvgdwrtagyndvggvssngvsvtrvsgngkdhvygsgdmgkkvvyvddhhkvhygtvdgvtnmdygrygavdgkktvtgtwngnkdrndgsrvtngvtgwrcraggggsvrgsggggssrhmgnsvvtyntndkcksgkakkmkgtsvvtyttdgckagdvdakmrskgvknvvtyttdgckagkvdadmkrgkdvvtyntdgcksgkakkmkgtsvvtyttdgckagdvdakmrskgvknvvtyttdgckagkvdadmkrgkdvvtyntdgcksgkakkmkgtsvvtyttdgckagdvdakmrskgvknvvtyttdgckagkvdadmkrgkdvvtyntdgcksgkakkmkgtsvvtyttdgckagdvdakmrskgvknvvtyttdgckagkvdadmkrgkdvvtyntdgcksgkakkmkgtsvvtyttdgckagdvdakmrskgvknvvtyttdgckagkvdadmkrgkdvvtyntdgcksgkakkmkgtsvvtyttdgckagdvdakmrskgvknvvtyttdgckagkvdadmkrgkdtyntsggkagrardvssgsggsgggghhhhhhgr

SEQ ID NO: 34 - DNA, 3190 bp, synthetic construct
  misc_difference 1..3190: note = NL_PPRcag_1
  misc_difference 2626: note = a, c, g, t or u
  misc_difference 2634: note = a, c, g, t or u
  misc_difference 2644: note = a, c, g, t or u
  misc_difference 2678: note = a, c, g, t or u
  misc_difference 2698: note = a, c, g, t or u
  misc_difference 2700: note = a, c, g, t or u
  misc_difference 2704: note = a, c, g, t or u
  misc_difference 2711: note = a, c, g, t or u
  misc_difference 2740: note = a, c, g, t or u
  misc_difference 2746: note = a, c, g, t or u
  misc_difference 2788: note = a, c, g, t or u
  misc_difference 2816: note = a, c, g, t or u
  misc_difference 2857: note = a, c, g, t or u
  misc_difference 2885: note = a, c, g, t or u
  misc_difference 2926: note = a, c, g, t or u
  misc_difference 2954: note = a, c, g, t or u
  misc_difference 2995: note = a, c, g, t or u
  misc_difference 3023: note = a, c, g, t or u
  misc_difference 3064: note = a, c, g, t or u
  misc_difference 3092: note = a, c, g, t or u
  misc_difference 3133: note = a, c, g, t or u
  misc_difference 3159: note = a, c, g, t or u
  source 1..3190: mol_type = other DNA
atggccggagtgttcacactggaggacttcgtgggcgactggagacagaccgccggctacaacctggaccaggtgctggagcagggcggagtgagcagcctgtttcagaacctgggcgtgagcgtcacccccatccagaggatcgtgctgtccggcgagaatggcctgaagatcgacatccacgtcatcatcccctacgagggcctgagcggcgatcagatgggccagatcgagaagatcttcaaggtggtgtatcccgtcgacgaccaccacttcaaggtgatcctgcattacggcaccctcgtgatcgacggcgtgacccctaacatgatcgactacttcggcaggccctacgagggaatcgccgtgttcgacggaaagaagatcaccgtgaccggcaccctgtggaacggaaacaagatcatcgacgagaggctgatcaaccccgacggctccctgctgttcagggtgaccatcaatggcgtgaccggctggagactgtgcgagagaatcctggccggaggcggaggaagcctcgtgcccagaggatccggcggaggcggctccagccggctccatatgggaaactccgtggtcacatacaacaccgatatcgacggcctgtgcaagagcggcaagatcgaggaggccctgaagctgttcaaggagatggaggagaagggcatcacccccagcgtggtcacatacaccacactgatcgacggactgtgtaaagccggcgacgtggacgaagccctcgagctgttcaaagagatgcggagcaagggcgtgaagcccaacgtggtgacatacaccaccctgatcgacggcctgtgcaaggccggcaaagtggacgaggccctggagctgttcgacgagatgaaggagaggggcatcaagcccgacgtggtcacatacaacaccctgatcgacggcctgtgcaagagcggcaagatcgaggaggccctgaagctgttcaaggagatggaggagaagggcatcacccccagcgtggttacatacaccacactgatcgacggactgtgtaaagccggcgacgtggacgaagccctcgagctgttcaaagagatgcggagcaagggcgtgaagcccaacgtggtcacatacaccaccctgatcgacggcctgtgcaaggccggcaaggtggatgaggccctggagctgttcgacgagatgaaggagaggggcatcaagcccgacgtggttacatacaacaccctgatcgacggcctgtgcaagagcggcaagatcgaggaggccctgaagctgttcaaggagatggaggagaagggcatcacccccagcgtggtcacatacaccacactgatcgacggactgtgtaaagccggcgacgtggacgaagccctcgagctgttcaaagagatgcggagcaagggcgtgaagcccaacgtggtgacatacaccaccctgatcgacggcctgtgcaaggccggcaaagtggacgaggccctggagctgttcgacgagatgaaggagaggggcatcaagcccgacgtggtcacatacaacaccctgatcgacggcctgtgcaagagcggcaagatcgaggaggccctgaagctgttcaaggagatggaggagaagggcatcacccccagcgtggttacatacaccacactgatcgacggactgtgtaaagccggcgacgtggacgaagccctcgagctgttcaaagagatgcggagcaagggcgtgaagcccaacgtggtcacatacaccaccctgatcgacggcctgtgcaaggccggcaaggtggatgaggccctggagctgttcgacgagatgaaggagaggggcatcaagcccgacgtggtcacatacaacaccctgatcgacggcctgtgcaagagcggcaagatcgaggaggccctgaagctgttcaaggagatggaggagaagggcatcacccccagcgtggtcacatacaccacactgatcgacggactgtgtaaagccggcgacgtggacgaagccctcgagctgttcaaagagatgcggagcaagggcgtgaagcccaacgtggttacatacaccaccctgatcgacggcctgtgcaaggccggcaaagtggacgaggccctggagctgttcgacgagatgaaggagaggggcatcaagcccgacgtggtcacatacaacaccctgatcgacggcctgtgcaagagcggcaagatcgaggaggccctgaagctgttcaaggagatggaggagaagggcatcacccccagcgtggtgacatacaccacactgatcgacggactgtgtaaagccggcgacgtggacgaagccctcgagctgttcaaagagatgcggagcaagggcgtgaagcccaacgtggtcacatacaccaccctgatcgacggcctgtgcaaggccggcaaggtggatgaggccctggagctgttcgacgagatgaaggagaggggcatcaagcccgacgagctgacctacaacaccctgatcagcggcctgggcaaggccggcagagccagagacccccccgtgctcagtagcggctccggcggcagcgggggaggcgggcatcatcaccatcaccacggacgctagmagvtdvgdwrtagyndvggvssngvsvtrvsgngkdhvygsgdmgkkvvyvddhhkvhygtvdgvtnmdygrygavdgkktvtgtwngnkdrndgsrvtngvtgwrcraggggsvrgsggggssrhmgnsvvtyntddgcksgkakkmkgtsvvtyttdgckagdvdakmrskgvknvvtyttdgckagkvdadmkrgkdvvtyntdgcksgkakkmkgtsvvtyttdgckagdvdakmrskgvknvvtyttdgckagkvdadmkrgkdvvtyntdgcksgkakkmkgtsvvtyttdgckagdvdakmrskgvknvvtyttdgckagkvdadmkrgkdvvtyntdgcksgkakkmkgtsvvtyttdgckagdvdakmrskgvknvvtyttdgckagkvdadmkrgkdvvtyntdgcksgkakkmkgtsvvtyttdgckagdvdakmrskgvknvvtyttdgckagkvdadmkrgkdvvtyntdgcksgkakkmkgtsvvtyttdgckagdvdakmrskgvknvvtyttdgckagkvdadmkrgkdtyntsggkagrardvssgsggsgggghhhhhhgr

SEQ ID NO: 35 - DNA, 1066 bp, synthetic construct
  misc_difference 1..1066: note = GFPnls
  misc_difference 878: note = a, c, g, t or u
  misc_difference 934: note = a, c, g, t or u
  misc_difference 946: note = a, c, g, t or u
  misc_difference 954: note = a, c, g, t or u
  misc_difference 959: note = a, c, g, t or u
  misc_difference 961: note = a, c, g, t or u
  misc_difference 964: note = a, c, g, t or u
  misc_difference 972: note = a, c, g, t or u
  misc_difference 976: note = a, c, g, t or u
  misc_difference 980: note = a, c, g, t or u
  misc_difference 989: note = a, c, g, t or u
  misc_difference 996: note = a, c, g, t or u
  misc_difference 1006: note = a, c, g, t or u
  source 1..1066: mol_type = other DNA
atggccggagtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagccaaagaaaaagagaaaggttagccatggctccggcggcagcgggggaggtagcggggactataaggaccacgacggagactacaaggatcatgatattgattacaaagacgatgacgataagatggccggacgctagmagvskgtgvvvdgdvnghksvsgggdatygktkcttgkvwtvtttygvcsrydhmkhdksamgyvrtkddgnyktravkgdtvnrkgdkdgnghkynynshnvymadkkngkvnkrhndgsvadhyntgdgvdnhystsaskdnkrdhmvvtaagtgmdykkkkrkvshgsggsgggsgdykdhdgdykdhddykddddkmagr

SEQ ID NO: 36 - DNA, 1170 bp, synthetic construct
  misc_difference 1..1170: note = mClover3nls
  misc_difference 959: note = a, c, g, t or u
  misc_difference 972: note = a, c, g, t or u
  misc_difference 1029: note = a, c, g, t or u
  misc_difference 1037: note = a, c, g, t or u
  misc_difference 1042..1043: note = a, c, g, t or u
  misc_difference 1054: note = a, c, g, t or u
  misc_difference 1058: note = a, c, g, t or u
  misc_difference 1062: note = a, c, g, t or u
  misc_difference 1072: note = a, c, g, t or u
  misc_difference 1079: note = a, c, g, t or u
  misc_difference 1089: note = a, c, g, t or u
  source 1..1170: mol_type = other DNA
atggccggagtgtccaaaggcgaggagctgtttaccggcgtcgtgcctattctggtggagctggacggcgacgtgaacggccacaagttctccgtgaggggcgagggcgaaggcgatgccacaaacggcaagctgaccctcaagttcatctgcaccactggtaaactgcccgttccttggcccacactggtgaccaccttcggctacggcgtggcttgtttctctcgttaccccgaccatatgaagcagcacgacttcttcaagtccgccatgcccgagggatacgtgcaagaaaggaccatctccttcaaggacgatggcacctacaagaccagagccgaggtgaagttcgagggcgacacactggtgaatcgtatcgaactgaagggcatcgacttcaaagaggacggcaacattctgggccacaagctggagtacaacttcaacagccactacgtgtacatcaccgccgataagcagaagaactgcatcaaggccaacttcaagattcgtcacaacgtggaggatggctccgtgcagctggccgatcactaccagcagaacacacccatcggcgatggacccgttttactgcccgacaaccactatttaagccaccagagcaagctgtccaaggaccccaacgagaagcgtgatcatatggtgctgctcgagtttgtgaccgccgccggcatcacccatggaatggacgagctgtacaagagccggctccatatgggatccggcggactcagtagccccaagaagaaacgcaaagtcgaggatccaaagaagaaaaggaaggttgaagaccccaagaaaaagaggaaggtgggttccgactataaggaccacgacggagactacaaggatcatgatattgattacaaagacgatgacgataagatggccccaaagaagaagcggaaggtcggacgctagmagvskgtgvvvdgdvnghksvrgggdatngktkcttgkvwtvttgygvacsrydhmkhdksamgyvrtskddgtyktravkgdtvnrkgdkdgnghkynnshyvytadkknckankrhnvdgsvadhyntgdgvdnhyshskskdnkrdhmvvtaagthgmdyksrhmgsggsskkkrkvdkkkrkvdkkkrkvgsdykdhdgdykdhddykddddkmakkkrkvgr

SEQ ID NO: 37 - DNA, 862 bp, synthetic construct
  misc_difference 1..862: note = NL control
  misc_difference 674: note = a, c, g, t or u
  misc_difference 730: note = a, c, g, t or u
  misc_difference 742: note = a, c, g, t or u
  misc_difference 750: note = a, c, g, t or u
  misc_difference 755: note = a, c, g, t or u
  misc_difference 757: note = a, c, g, t or u
  misc_difference 760: note = a, c, g, t or u
  misc_difference 768: note = a, c, g, t or u
  misc_difference 772: note = a, c, g, t or u
  misc_difference 776: note = a, c, g, t or u
  misc_difference 785: note = a, c, g, t or u
  misc_difference 792: note = a, c, g, t or u
  misc_difference 802: note = a, c, g, t or u
  source 1..862: mol_type = other DNA
atggccggagtgttcacactggaggacttcgtgggcgactggagacagaccgccggctacaacctggaccaggtgctggagcagggcggagtgagcagcctgtttcagaacctgggcgtgagcgtcacccccatccagaggatcgtgctgtccggcgagaatggcctgaagatcgacatccacgtcatcatcccctacgagggcctgagcggcgatcagatgggccagatcgagaagatcttcaaggtggtgtatcccgtcgacgaccaccacttcaaggtgatcctgcattacggcaccctcgtgatcgacggcgtgacccctaacatgatcgactacttcggcaggccctacgagggaatcgccgtgttcgacggaaagaagatcaccgtgaccggcaccctgtggaacggaaacaagatcatcgacgagaggctgatcaaccccgacggctccctgctgttcagggtgaccatcaatggcgtgaccggctggagactgtgcgagagaatcctggccggaggcggaggaagcctcgtgcccagaggatccggcggaggcggctccagccggctccatatgggatccggcggactcagtagcggctccggcggcagcgggggaggcgggcatcatcaccatcaccacggacgctagmagvskgtgvvvdgdvnghksvsgggdatygktkcttgkvwtvtttygvcsrydhmkhdksamgyvrtkddgnyktravkgdtvnrkgdkdgnghkynynshnvymadkkngkvnkrhndgsvadhyntgdgvdnhystsaskdnkrdhmvvtaagtgmdykkkkrkvshgsggsgggsgdykdhdgdykdhddykddddkmagr

SEQ ID NO: 38 - DNA, 30 bp, synthetic construct
  misc_feature 1..30: note = RNA probe
  source 1..30: mol_type = other DNA
gacatgccagcagcagcagcagcaggactg

SEQ ID NO: 39 - DNA, 30 bp, synthetic construct
  misc_feature 1..30: note = RNA probe
  source 1..30: mol_type = other DNA
gacatgccggcggcggcggcggcgggactg

SEQ ID NO: 40 - DNA, 30 bp, synthetic construct
  misc_feature 1..30: note = RNA probe
  source 1..30: mol_type = other DNA
gacactgctgctgctgctgctgctgatgca

SEQ ID NO: 41 - DNA, 30 bp, synthetic construct
  misc_feature 1..30: note = RNA probe
  source 1..30: mol_type = other DNA
gacaccgccgccgccgccgccgccggactg

SEQ ID NO: 42 - DNA, 30 bp, synthetic construct
  misc_feature 1..30: note = RNA probe
  source 1..30: mol_type = other DNA
gacatgctggtgtatcttgtctttagactg

SEQ ID NO: 43 - DNA, 63 bp, synthetic construct
  misc_feature 1..63: note = Dest-a
  source 1..63: mol_type = other DNA
gaagacataaactccgtggtcacatacagagaccaaggtctcagtggtcacatacatgtcttc

SEQ ID NO: 44 - DNA, 47 bp, synthetic construct
  misc_feature 1..47: note = Dest-b
  source 1..47: mol_type = other DNA
gaagacatatacagagaccaaggtctcagtggtgacataatgtcttc

SEQ ID NO: 45 - DNA, 47 bp, synthetic construct
  misc_feature 1..47: note = Dest-c
  source 1..47: mol_type = other DNA
gaagacatcatacagagaccaaggtctcagtggttacatatgtcttc

SEQ ID NO: 46 - DNA, 47 bp, synthetic construct
  misc_feature 1..47: note = Dest-d
  source 1..47: mol_type = other DNA
gaagacatacatacagagaccaaggtctcagtggttacaatgtcttc

SEQ ID NO: 47 - DNA, 47 bp, synthetic construct
  misc_feature 1..47: note = Dest-e
  source 1..47: mol_type = other DNA
gaagacattacatacagagaccaaggtctcagtggtgacatgtcttc

SEQ ID NO: 48 - DNA, 47 bp, synthetic construct
  misc_feature 1..47: note = Dest-f
  source 1..47: mol_type = other DNA
gaagacattgacatacagagaccaaggtctcagtggttaatgtcttc

SEQ ID NO: 49 - DNA, 47 bp, synthetic construct
  misc_feature 1..47: note = Dest-g
  source 1..47: mol_type = other DNA
gaagacatgttacatacagagaccaaggtctcagtggtcatgtcttc

SEQ ID NO: 50 - DNA, 47 bp, synthetic construct
  misc_feature 1..47: note = Dest-h
  source 1..47: mol_type = other DNA
gaagacatggtcacatacagagaccaaggtctcagtggtatgtcttc

SEQ ID NO: 51 - DNA, 47 bp, synthetic construct
  misc_feature 1..47: note = Dest-i
  source 1..47: mol_type = other DNA
gaagacattggttacatacagagaccaaggtctcagtggatgtcttc

SEQ ID NO: 52 - DNA, 45 bp, synthetic construct
  misc_feature 1..45: note = Dest-j
  source 1..45: mol_type = other DNA
gaagacatgtggtgacatacagagaccaaggtctcagtggtcttc

SEQ ID NO: 53 - protein, 5 aa, synthetic construct
  REGION 1..5: note = signal sequence
MGNSV

SEQ ID NO: 54 - protein, 22 aa, synthetic construct
  REGION 1..22: note = signal sequence
ELTYNTLISGLGKAGRARDPPV

SEQ ID NO: 55 - protein, 4 aa, synthetic construct
  REGION 1..4: note = signal sequence
KDEL

SEQ ID NO: 56 - protein, 4 aa, synthetic construct
  REGION 1..4: note = signal sequence
KEEL

SEQ ID NO: 57 - protein, 19 aa, synthetic construct
  REGION 1..19: note = signal sequence
MGWSCIILFLVATATGAHS

SEQ ID NO: 58 - protein, 35 aa, synthetic construct
  REGION 1..35: note = 1st_A (v3.2_A)
VTYTTNIDQLCKAGKVDEALELFKEMRSKGVKPNV

SEQ ID NO: 59 - protein, 35 aa, synthetic construct
  REGION 1..35: note = 1st_G (v3.2_G)
VTYTTNIDQLCKAGKVDEALELFDEMKERGIKPDV

SEQ ID NO: 60 - protein, 35 aa, synthetic construct
  REGION 1..35: note = 1st_U (v3.2_U)
VTYNTNIDQLCKAGRLDEAEELLEEMEEKGIKPDV

SEQ ID NO: 61 - DNA, 2271 bp, synthetic construct
  misc_feature 1..2271: note = v2_polypucleotide
  source 1..2271: mol_type = other DNA
atgggtcatcaccatcatcatcacgggtccctgcaggactcagaagtcaatcaagaagctaagccagaggtcaagccagaagtcaagcctgagactcacatcaatttaaaggtgtccgatggatcttcagagatcttcttcaagatcaaaaagaccactcctttaagaaggctgatggaagcgttcgctaaaagacagggtaaggaaatggactccttaagattcttgtacgacggtattagaattcaagctgatcaggcccctgaagatttggacatggaggataacgatattattgaggctcaccgcgaacagattggaggtatgggaaactccgtggtcacatacaacaccctgatcgacggcctgtgcaagagcggcaagatcgaggaggccctgaagctgttcaaggagatggaggagaagggcatcacccccagcgtggtcacatacaccacactgatcgacggactgtgtaaagccggcgacgtggacgaagccctcgagctgttcaaagagatgcggagcaagggcgtgaagcccaacgtggtgacatacaccaccctgatcgacggcctgtgcaaggccggcaaagtggacgaggccctggagctgttcgacgagatgaaggagaggggcatcaagcccgacgtggtcacatacaacaccctgatcgacggcctgtgcaagagcggcaagatcgaggaggccctgaagctgttcaaggagatggaggagaagggcatcacccccagcgtggttacatacaccacactgatcgacggactgtgtaaagccggcgacgtggacgaagccctcgagctgttcaaagagatgcggagcaagggcgtgaagcccaacgtggtcacatacaccaccctgatcgacggcctgtgcaaggccggcaaggtggatgaggccctggagctgttcgacgagatgaaggagaggggcatcaagcccgacgtggttacatacaacaccctgatcgacggcctgtgcaagagcggcaagatcgaggaggccctgaagctgttcaaggagatggaggagaagggcatcacccccagcgtggtcacatacaccacactgatcgacggactgtgtaaagccggcgacgtggacgaagccctcgagctgttcaaagagatgcggagcaagggcgtgaagcccaacgtggtgacatacaccaccctgatcgacggcctgtgcaaggccggcaaagtggacgaggccctggagctgttcgacgagatgaaggagaggggcatcaagcccgacgtggtcacatacaacaccctgatcgacggcctgtgcaagagcggcaagatcgaggaggccctgaagctgttcaaggagatggaggagaagggcatcacccccagcgtggttacatacaccacactgatcgacggactgtgtaaagccggcgacgtggacgaagccctcgagctgttcaaagagatgcggagcaagggcgtgaagcccaacgtggtcacatacaccaccctgatcgacggcctgtgcaaggccggcaaggtggatgaggccctggagctgttcgacgagatgaaggagaggggcatcaagcccgacgtggtcacatacaacaccctgatcgacggcctgtgcaagagcggcaagatcgaggaggccctgaagctgttcaaggagatggaggagaagggcatcacccccagcgtggtcacatacaccacactgatcgacggactgtgtaaagccggcgacgtggacgaagccctcgagctgttcaaagagatgcggagcaagggcgtgaagcccaacgtggttacatacaccaccctgatcgacggcctgtgcaaggccggcaaagtggacgaggccctggagctgttcgacgagatgaaggagaggggcatcaagcccgacgtggtcacatacaacaccctgatcgacggcctgtgcaagagcggcaagatcgaggaggccctgaagctgttcaaggagatggaggagaagggcatcacccccagcgtggtgacatacaccacactgatcgacggactgtgtaaagccggcgacgtggacgaagccctcgagctgttcaaagagatgcggagcaagggcgtgaagcccaacgtggtcacatacaccaccctgatcgacggcctgtgcaaggccggcaaggtggatgaggccctggagctgttcgacgagatgaaggagaggggcatcaagcccgacgagctgacctacaacaccctgatcagcggcctgggcaaggccggc

SEQ ID NO: 62 - protein, 757 aa, synthetic construct
  REGION 1..757: note = v2_protein
MGHHHHHHGSLQDSEVNQEAKPEVKPEVKPETHINLKVSDGSSEIFFKIKKTTPLRRLMEAFAKRQGKEMDSLRFLYDGIRIQADQAPEDLDMEDNDIIEAHREQIGGMGNSVVTYNTLIDGLCKSGKIEEALKLFKEMEEKGITPSVVTYTTLIDGLCKAGDVDEALELFKEMRSKGVKPNVVTYTTLIDGLCKAGKVDEALELFDEMKERGIKPDVVTYNTLIDGLCKSGKIEEALKLFKEMEEKGITPSVVTYTTLIDGLCKAGDVDEALELFKEMRSKGVKPNVVTYTTLIDGLCKAGKVDEALELFDEMKERGIKPDVVTYNTLIDGLCKSGKIEEALKLFKEMEEKGITPSVVTYTTLIDGLCKAGDVDEALELFKEMRSKGVKPNVVTYTTLIDGLCKAGKVDEALELFDEMKERGIKPDVVTYNTLIDGLCKSGKIEEALKLFKEMEEKGITPSVVTYTTLIDGLCKAGDVDEALELFKEMRSKGVKPNVVTYTTLIDGLCKAGKVDEALELFDEMKERGIKPDVVTYNTLIDGLCKSGKIEEALKLFKEMEEKGITPSVVTYTTLIDGLCKAGDVDEALELFKEMRSKGVKPNVVTYTTLIDGLCKAGKVDEALELFDEMKERGIKPDVVTYNTLIDGLCKSGKIEEALKLFKEMEEKGITPSVVTYTTLIDGLCKAGDVDEALELFKEMRSKGVKPNVVTYTTLIDGLCKAGKVDEALELFDEMKERGIKPDELTYNTLISGLGKAG

SEQ ID NO: 63 - DNA, 2274 bp, synthetic construct
  misc_feature 1..2274: note = v3.2_polypucleotide
  source 1..2274: mol_type = other DNA
atgggtcatcaccatcatcatcacgggtccctgcaggactcagaagtcaatcaagaagctaagccagaggtcaagccagaagtcaagcctgagactcacatcaatttaaaggtgtccgatggatcttcagagatcttcttcaagatcaaaaagaccactcctttaagaaggctgatggaagcgttcgctaaaagacagggtaaggaaatggactccttaagattcttgtacgacggtattagaattcaagctgatcaggcccctgaagatttggacatggaggataacgatattattgaggctcaccgcgaacagattggaggtatgggaaactccgtggtcacatacaacaccaacatcgaccagctgtgcaagagcggcaagatcgaggaggccctgaagctgttcaaggagatggaggagaagggcatcacccccagcgtggtcacatacaccacactgatcgacggactgtgtaaagccggcgacgtggacgaagccctcgagctgttcaaagagatgcggagcaagggcgtgaagcccaacgtggtgacatacaccaccctgatcgacggcctgtgcaaggccggcaaagtggacgaggccctggagctgttcgacgagatgaaggagaggggcatcaagcccgacgtggtcacatacaacaccctgatcgacggcctgtgcaagagcggcaagatcgaggaggccctgaagctgttcaaggagatggaggagaagggcatcacccccagcgtggttacatacaccacactgatcgacggactgtgtaaagccggcgacgtggacgaagccctcgagctgttcaaagagatgcggagcaagggcgtgaagcccaacgtggtcacatacaccaccctgatcgacggcctgtgcaaggccggcaaggtggatgaggccctggagctgttcgacgagatgaaggagaggggcatcaagcccgacgtggttacatacaacaccctgatcgacggcctgtgcaagagcggcaagatcgaggaggccctgaagctgttcaaggagatggaggagaagggcatcacccccagcgtggtcacatacaccacactgatcgacggactgtgtaaagccggcgacgtggacgaagccctcgagctgttcaaagagatgcggagcaagggcgtgaagcccaacgtggtgacatacaccaccctgatcgacggcctgtgcaaggccggcaaagtggacgaggccctggagctgttcgacgagatgaaggagaggggcatcaagcccgacgtggtcacatacaacaccctgatcgacggcctgtgcaagagcggcaagatcgaggaggccctgaagctgttcaaggagatggaggagaagggcatcacccccagcgtggttacatacaccacactgatcgacggactgtgtaaagccggcgacgtggacgaagccctcgagctgttcaaagagatgcggagcaagggcgtgaagcccaacgtggtcacatacaccaccctgatcgacggcctgtgcaaggccggcaaggtggatgaggccctggagctgttcgacgagatgaaggagaggggcatcaagcccgacgtggtcacatacaacaccctgatcgacggcctgtgcaagagcggcaagatcgaggaggccctgaagctgttcaaggagatggaggagaagggcatcacccccagcgtggtcacatacaccacactgatcgacggactgtgtaaagccggcgacgtggacgaagccctcgagctgttcaaagagatgcggagcaagggcgtgaagcccaacgtggttacatacaccaccctgatcgacggcctgtgcaaggccggcaaagtggacgaggccctggagctgttcgacgagatgaaggagaggggcatcaagcccgacgtggtcacatacaacaccctgatcgacggcctgtgcaagagcggcaagatcgaggaggccctgaagctgttcaaggagatggaggagaagggcatcacccccagcgtggtgacatacaccacactgatcgacggactgtgtaaagccggcgacgtggacgaagccctcgagctgttcaaagagatgcggagcaagggcgtgaagcccaacgtggtcacatacaccaccctgatcgacggcctgtgcaaggccggcaaggtggatgaggccctggagctgttcgacgagatgaaggagaggggcatcaagcccgacgaggagctgacctacaacaccctgatcagcggcctgggcaaggccggc

SEQ ID NO: 64 - protein, 757 aa, synthetic construct
  REGION 1..757: note = v3.2_protein
MGHHHHHHGSLQDSEVNQEAKPEVKPEVKPETHINLKVSDGSSEIFFKIKKTTPLRRLMEAFAKRQGKEMDSLRFLYDGIRIQADQAPEDLDMEDNDIIEAHREQIGGMGNSVVTYNTNIDQLCKSGKIEEALKLFKEMEEKGITPSVVTYTTLIDGLCKAGDVDEALELFKEMRSKGVKPNVVTYTTLIDGLCKAGKVDEALELFDEMKERGIKPDVVTYNTLIDGLCKSGKIEEALKLFKEMEEKGITPSVVTYTTLIDGLCKAGDVDEALELFKEMRSKGVKPNVVTYTTLIDGLCKAGKVDEALELFDEMKERGIKPDVVTYNTLIDGLCKSGKIEEALKLFKEMEEKGITPSVVTYTTLIDGLCKAGDVDEALELFKEMRSKGVKPNVVTYTTLIDGLCKAGKVDEALELFDEMKERGIKPDVVTYNTLIDGLCKSGKIEEALKLFKEMEEKGITPSVVTYTTLIDGLCKAGDVDEALELFKEMRSKGVKPNVVTYTTLIDGLCKAGKVDEALELFDEMKERGIKPDVVTYNTLIDGLCKSGKIEEALKLFKEMEEKGITPSVVTYTTLIDGLCKAGDVDEALELFKEMRSKGVKPNVVTYTTLIDGLCKAGKVDEALELFDEMKERGIKPDVVTYNTLIDGLCKSGKIEEALKLFKEMEEKGITPSVVTYTTLIDGLCKAGDVDEALELFKEMRSKGVKPNVVTYTTLIDGLCKAGKVDEALELFDEMKERGIKPDELTYNTLISGLGKAG

(19) United States
(12) Patent Application Publication
(10) Pub. No.: US 2026/0317383 A1
(43) Pub. Date: Oct. 15, 2026

(54) PPR PROTEIN CAUSING LESS AGGREGATION AND USE OF THE SAME

(71) Applicants: EditForce, Inc., Fukuoka City (JP); KYUSHU UNIVERSITY, NATIONAL UNIVERSITY CORPORATION, Fukuoka-shi (JP)

(72) Inventors: Yusuke Yagi, Fukuoka City (JP); Takayoshi Imai, Fukuoka City (JP); Takayuki Tamai, Fukuoka City (JP); Takahiro Nakamura, Fukuoka-shi (JP); Takamasa Teramoto, Fukuoka-shi (JP)

(73) Assignees: EditForce, Inc., Fukuoka City (JP); KYUSHU UNIVERSITY, NATIONAL UNIVERSITY CORPORATION, Fukuoka-shi (JP)

(21) Appl. No.: 19/455,190

(22) Filed: Jan. 21, 2026

Related U.S. Application Data
(62) Division of application No. 17/614,236, filed on May 29, 2020, now Pat. No. 12,559,528.

(30) Foreign Application Priority Data
May 29, 2019 (JP) 2019-100553

(51) Int. Cl. C07K 14/46; C07K 7/06; C07K 7/08; C07K 19/00; C12N 5/10; C12N 15/63

(52) U.S. Cl. CPC C07K 14/46; C07K 7/06; C07K 7/08; C07K 19/00; C12N 5/10; C12N 15/63

(57) ABSTRACT

In order to improve aggregation property of a PPR protein, the A₆ amino acid of the 1st PPR motif from the N-terminus (M₁) is made more hydrophilic. Further, the A₉ amino acid of M₁ is made to be a hydrophilic amino acid or glycine. The A₆ amino acid is preferably asparagine or aspartic acid, and the A₉ amino acid is preferably glutamine, glutamic acid, lysine, or glycine. Proteins containing such a PPR motif as M₁ motif may have not only improved aggregation property, but also high binding power to a target nucleic acid.

### CROSS-REFERENCE TO RELATED APPLICATIONS

This application is a divisional of U.S. application Ser. No. 17/614,236, filed on Nov. 24, 2021, which is a 371 of International Application No. PCT/JP2020/021473 filed on May 29, 2020, which is based upon and claims the benefit of priority from the prior Japanese Patent Application No. 2019-100553, filed on May 29, 2019, the entire contents of which are incorporated herein by reference.

### SEQUENCE LISTING

The sequence listing that is contained in the file named is “P211027US01_Replacement Sequence_Listing.xml” having a size of 381,981 bytes and created on Jun. 16, 2026, is incorporated herein by reference in its entirety.

### TECHNICAL FIELD

The present invention relates to a nucleic acid manipulation technique using a protein capable of binding to an intended nucleic acid. The present invention is useful in a wide range of fields, including medicine (drug discovery support, therapeutic treatment etc.), agriculture (agricultural, fishery and livestock production, breeding etc.), and chemistry (biological material production etc.).

### BACKGROUND TECHNIQUES

PPR proteins are proteins comprising repeat of PPR motifs each having about 35 amino acids length, and one PPR motif can specifically bind to one base. The combination of the first, fourth, and ii-th (second from the end before the next motif) amino acids in a PPR motif determines to which one of adenine, cytosine, guanine, and uracil (or thymine) the motif binds (Patent documents 1 and 2).

Among the naturally occurring RNA-binding PPR motifs, the combinations corresponding to each of the bases most frequently occurring are: first valine, fourth threonine, and ii-th asparagine for adenine; first valine, fourth asparagine, and ii-th serine for cytosine; first valine, fourth threonine, and ii-th aspartic acid for guanine, and first valine, fourth asparagine, and ii-th aspartic acid for uracil (Non-patent documents 1 to 5). By using these combinations of amino acids, PPR proteins that can specifically bind to an arbitrary sequence can be designed.

### PRIOR ART REFERENCES

### Patent Documents

Patent document 1: International Publication WO2013/058404
Patent document 2: International Publication WO2014/175284
Patent document 3: Japanese Patent Application No. 2019-100551

### Non-Patent Documents

Non-patent document 1: Coquille, S. et al., An artificial PPR scaffold for programmable RNA recognition, Nature Communications 5, Article number: 5729 (2014)
Non-patent document 2: Shen, C. et al., Specific RNA Recognition by Designer Pentatricopeptide Repeat Protein, Molecular Plant 8, 667-670 (2015)
Non-patent document 3: Shen, C. et al., Structural basis for specific single-stranded RNA recognition by designer pentatricopeptide repeat proteins, Nature Communications, Volume 7, Article number: 11285 (2016)
Non-patent document 4: Miranda, R. G. et al., RNA-binding specificity landscapes of designer pentatricopeptide repeat proteins elucidate principles of PPR-RNA interactions, Nucleic Acids Research, 46(5), 2613-2623 (2018)
Non-patent document 5: Yan, J. et al., Delineation of pentatricopeptide repeat codes for target RNA prediction, Nucleic Acids Research, gkz075 (2019)

### SUMMARY OF THE INVENTION

### Object to be Achieved by the Invention

The inventors of the present invention have examined preparation of PPR proteins having high performance and formed by linking many, for example, 15 or more, of PPR motifs using the amino acid combinations mentioned above (Patent Document 3). On the other hand, according to the examination of the inventors of the present invention, it was found that some of the PPR proteins prepared in such a manner show aggregation property. In particular, when the PPR proteins were expressed in cultured animal cells, aggregation may be observed.

### Means for Achieving the Object

Therefore, the inventors of the present invention examined to solve this problem by amino acid mutation in the PPR motifs. Then, they found that the aggregation properties of PPR proteins can be improved by changing the 6th, preferably the 6th and 9th, amino acids of the first motif (on the N-terminal side) of the PPR protein to hydrophilic amino acids, and accomplished the present invention.

The present invention provides the followings.
[1] A PPR motif, which is any one of the following PPR motifs:
(C-1) a PPR motif consisting of any one of the sequences of SEQ ID NOS: 4 to 7;
(C-2) a PPR motif consisting of any one of the sequences of SEQ ID NOS: 4 to 7 having a substitution, deletion, or addition of 1 to 9 amino acids other than the amino acids at positions 1, 4, 6, and 34, and having a cytosine-binding property;
(C-3) a PPR motif having a sequence identity of at least 80% to any one of the sequences of SEQ ID NOS: 4 to 7, provided that the amino acids at positions 1, 4, 6, and 34 are identical, and having a cytosine-binding property;
(A-1) a PPR motif consisting of the sequence of SEQ ID NO: 8 having a substitution of the amino acid at position 6 with asparagine or aspartic acid;
(A-2) a PPR motif consisting of the sequence of (A-1) having a substitution, deletion, or addition of 1 to 9 amino acids other than the amino acids at positions 1, 4, 6, and 34, and having an adenine-binding property;
(A-3) a PPR motif having a sequence identity of at least 80% to the sequence of (A-1), provided that the amino acids at positions 1, 4, 6, and 34 are identical, and having an adenine-binding property;
(G-1) a PPR motif consisting of the sequence of SEQ ID NO: 9 having a substitution of the amino acid at position 6 with asparagine or aspartic acid;
(G-2) a PPR motif consisting of the sequence of (G-1) having a substitution, deletion, or addition of 1 to 9 amino acids other than the amino acids at positions 1, 4, 6, and 34, and having a guanine-binding property;
(G-3) a PPR motif having a sequence identity of at least 80% to the sequence of (G-1), provided that the amino acids at positions 1, 4, 6, and 34 are identical, and having a guanine-binding property;
(U-1) a PPR motif consisting of the sequence of SEQ ID NO: 10 having a substitution of the amino acid at position 6 with asparagine or aspartic acid;
(U-2) a PPR motif consisting of the sequence of (U-1) having a substitution, deletion, or addition of 1 to 9 amino acids other than the amino acids at positions 1, 4, 6, and 34, and having a uracil-binding property; and
(U-3) a PPR motif having a sequence identity of at least 80% to the sequence of (U-1), provided that the amino acids at positions 1, 4, 6, and 34 are identical, and having a uracil-binding property.
[2] A PPR motif, which is any one of the following PPR motifs:
(C-1) a PPR motif consisting of any one of the sequences of SEQ ID NOS: 4 to 7;
(C-2) a PPR motif consisting of any one of the sequences of SEQ ID NOS: 4 to 7 having a substitution, deletion, or addition of 1 to 9 amino acids other than the amino acids at positions 1, 4, 6, 9, and 34, and having a cytosine-binding property;
(C-3) a PPR motif having a sequence identity of at least 80% to any one of the sequences of SEQ ID NOS: 4 to 7, provided that the amino acids at positions 1, 4, 6, 9, and 34 are identical, and having a cytosine-binding property;
(A-1) a PPR motif consisting of the sequence of SEQ ID NO: 8 having such substitutions of the amino acids at positions 6 and 9 that any one of the combinations defined below is satisfied;
(A-2) a PPR motif consisting of the sequence of (A-1) having a substitution, deletion, or addition of 1 to 9 amino acids other than the amino acids at positions 1, 4, 6, 9, and 34, and having an adenine-binding property;
(A-3) a PPR motif having a sequence identity of at least 80% to the sequence of (A-1), provided that the amino acids at positions 1, 4, 6, 9, and 34 are identical, and having an adenine-binding property;
(G-1) a PPR motif consisting of the sequence of SEQ ID NO: 9 having such substitutions of the amino acids at positions 6 and 9 that any one of the combinations defined below is satisfied;
(G-2) a PPR motif consisting of the sequence of (G-1) having a substitution, deletion, or addition of 1 to 9 amino acids other than the amino acids at positions 1, 4, 6, 9, and 34, and having a guanine-binding property;
(G-3) a PPR motif having a sequence identity of at least 80% to the sequence of (G-1), provided that the amino acids at positions 1, 4, 6, 9, and 34 are identical, and having a guanine-binding property;
(U-1) a PPR motif consisting of the sequence of SEQ ID NO: 10 having such substitutions of the amino acids at positions 6 and 9 that any one of the combinations defined below is satisfied;
(U-2) a PPR motif consisting of the sequence of (U-1) having a substitution, deletion, or addition of 1 to 9 amino acids other than the amino acids at positions 1, 4, 6, 9, and 34, and having a uracil-binding property; and
(U-3) a PPR motif having a sequence identity of at least 80% to the sequence of (U-1), provided that the amino acids at positions 1, 4, 6, 9, and 34 are identical, and having a uracil-binding property.
   a combination of asparagine as the amino acid at position 6 and glutamic acid as the amino acid at position 9,
   a combination of asparagine as the amino acid at position 6 and glutamine as the amino acid at position 9,
   a combination of asparagine as the amino acid at position 6 and lysine as the amino acid at position 9, and
   a combination of aspartic acid as the amino acid at position 6 and glycine as the amino acid at position 9.
[3] The PPR motif according to 1 or 2, which is any one of the following PPR motifs:
(C-4) a PPR motif consisting of the sequence of SEQ ID NO: 4;
(A-4) a PPR motif consisting of the sequence of SEQ ID NO: 58;
(G-4) a PPR motif consisting of the sequence of SEQ ID NO: 59; and
(U-4) a PPR motif consisting of the sequence of SEQ ID NO: 60.
[4] Use of the PPR motif according to any one of 1 to 3 in a PPR protein as the first PPR motif from the N-terminus.
[5] The use according to 4, which is for reducing aggregation of the PPR protein.
[6] A protein capable of binding to a target nucleic acid having a specific nucleotide sequence, which comprises 1 to 30 of PPR motifs represented by the formula 1 mentioned below, and wherein the A₆ amino acid of the first PPR motif (M₁) from the N-terminus is a hydrophilic amino acid:

[Formula 1]

(Helix A)-X-(Helix B)-L (Formula 1)
wherein, in the formula:
   Helix A is a moiety of 12-amino acid length capable of forming an α-helix structure, and is represented by the formula 2;

[Formula 2]

A₁-A₂-A₃-A₄-A₅-A₆-A₇-A₈-A₉-A₁₀-A₁₁-A₁₂ (Formula 2)
wherein, in the formula 2, A₁ to A₁₂ independently represent an amino acid;
   X does not exist, or is a moiety of 1- to 9-amino acid length;
   Helix B is a moiety of 11- to 13-amino acid length capable of forming an α-helix structure; and
   L is a moiety of 2- to 7-amino acid length represented by the formula 3;

[Formula 3]

Lᵥᵢᵢ-Lᵥᵢ-Lᵥ-Lᵢᵥ-Lᵢᵢᵢ-Lᵢᵢ-Lᵢ (Formula 3)
wherein, in the formula 3, the amino acids are numbered “i” (-1), “ii” (-2), and so on from the C-terminus side,
   provided that Lᵢᵢᵢ to Lᵥᵢᵢ may not exist.
[7] The protein according to 6, wherein the A₉ amino acid of M₁ is a hydrophilic amino acid or glycine.
[8] The protein according to 6 or 7, wherein the A₆ amino acid of M₁ is asparagine or aspartic acid.
[9] The protein according to any one of 6 to 8, wherein the A₉ amino acid of M₁ is glutamine, glutamic acid, lysine, or glycine.
[10] The protein according to any one of 6 to 9, wherein the A₆ amino acid of M₁ and the A₉ amino acid of M₁ correspond to any of the following combinations:
   combination of asparagine as the A₆ amino acid and glutamic acid as the A₉ amino acid,
   combination of asparagine as the A₆ amino acid and glutamine as the A₉ amino acid,
   combination of asparagine as the A₆ amino acid and lysine as the A₉ amino acid, and
   combination of aspartic acid as the A₆ amino acid and glycine as the A₉ amino acid.
[11] A fusion protein of at least one selected from the group consisting of a fluorescent protein, a nuclear localization signal peptide, and a tag protein, and a PPR protein containing the PPR motif according to any one of 1 to 3 as the first PPR motif from the N-terminus, or the protein according to any one of 6 to 10.
[12] A method for modifying a PPR protein containing the PPR motif according to 6 and capable of binding to a target nucleic acid having a specific nucleotide sequence, which comprises making the A₆ amino acid of the first PPR motif (M₁) from the N-terminus more hydrophilic.
[13] A method for detecting a nucleic acid, which uses a PPR protein containing the PPR motif according to any one of 1 to 3 as the first PPR motif from the N-terminus, the protein according to any one of 6 to 10, or the fusion protein according to 11.
[14] A nucleic acid encoding the PPR motif according to any one of 1 to 3, a PPR protein containing the PPR motif according to any one of 1 to 3 as the first PPR motif from the N-terminus, or the protein according to any one of 6 to 10.
[15]A vector comprising the nucleic acid according to 14.
[16] A cell (except for human individual) containing the vector according to 15.
[17] A method for manipulating a nucleic acid, which uses the PPR motif according to any one of 1 to 3, a PPR protein containing the PPR motif according to any one of 1 to 3 as the first PPR motif from the N-terminus, the protein according to any one of 6 to 10, or the vector according to 15 (implementation in human individual is excluded).
[18] A method for producing an organism, which comprises the manipulation method according to 17.

The present invention also provides the followings.
[1] A PPR motif, which is any one of the following PPR motifs:
(C-1) a PPR motif consisting of any one of the sequences of SEQ ID NOS: 4 to 7;
(C-2) a PPR motif consisting of any one of the sequences of SEQ ID NOS: 4 to 7 having a substitution, deletion, or addition of 1 to 9 amino acids other than the amino acids at positions 1, 4, 6, and 34, and having a cytosine-binding property;
(C-3) a PPR motif having a sequence identity of at least 80% to any one of the sequences of SEQ ID NOS: 4 to 7, provided that the amino acids at positions 1, 4, 6, and 34 are identical, and having a cytosine-binding property;
(A-1) a PPR motif consisting of the sequence of SEQ ID NO: 8 having a substitution of the amino acid at position 6 with asparagine or aspartic acid;
(A-2) a PPR motif consisting of the sequence of (A-1) having a substitution, deletion, or addition of 1 to 9 amino acids other than the amino acids at positions 1, 4, 6, and 34, and having an adenine-binding property;
(A-3) a PPR motif having a sequence identity of at least 80% to the sequence of (A-1), provided that the amino acids at positions 1, 4, 6, and 34 are identical, and having an adenine-binding property;
(G-1) a PPR motif consisting of the sequence of SEQ ID NO: 9 having a substitution of the amino acid at position 6 with asparagine or aspartic acid;
(G-2) a PPR motif consisting of the sequence of (G-1) having a substitution, deletion, or addition of 1 to 9 amino acids other than the amino acids at positions 1, 4, 6, and 34, and having a guanine-binding property;
(G-3) a PPR motif having a sequence identity of at least 80% to the sequence of (G-1), provided that the amino acids at positions 1, 4, 6, and 34 are identical, and having a guanine-binding property;
(U-1) a PPR motif consisting of the sequence of SEQ ID NO: 10 having a substitution of the amino acid at position 6 with asparagine or aspartic acid;
(U-2) a PPR motif consisting of the sequence of (U-1) having a substitution, deletion, or addition of 1 to 9 amino acids other than the amino acids at positions 1, 4, 6, and 34, and having a uracil-binding property; and
(U-3) a PPR motif having a sequence identity of at least 80% to the sequence of (U-1), provided that the amino acids at positions 1, 4, 6, and 34 are identical, and having a uracil-binding property.
[2] A PPR motif, which is any one of the following PPR motifs:
(C-1) a PPR motif consisting of any one of the sequences of SEQ ID NOS: 4 to 7;
(C-2) a PPR motif consisting of any one of the sequences of SEQ ID NOS: 4 to 7 having a substitution, deletion, or addition of 1 to 9 amino acids other than the amino acids at positions 1, 4, 6, 9, and 34, and having a cytosine-binding property;
(C-3) a PPR motif having a sequence identity of at least 80% to any one of the sequences of SEQ ID NOS: 4 to 7, provided that the amino acids at positions 1, 4, 6, 9, and 34 are identical, and having a cytosine-binding property;
(A-1) a PPR motif consisting of the sequence of SEQ ID NO: 8 having such substitutions of the amino acids at positions 6 and 9 that any one of the combinations defined below is satisfied;
(A-2) a PPR motif consisting of the sequence of (A-1) having a substitution, deletion, or addition of 1 to 9 amino acids other than the amino acids at positions 1, 4, 6, 9, and 34, and having an adenine-binding property;
(A-3) a PPR motif having a sequence identity of at least 80% to the sequence of (A-1), provided that the amino acids at positions 1, 4, 6, 9, and 34 are identical, and having an adenine-binding property;
(G-1) a PPR motif consisting of the sequence of SEQ ID NO: 9 having such substitutions of the amino acids at positions 6 and 9 that any one of the combinations defined below is satisfied;
(G-2) a PPR motif consisting of the sequence of (G-1) having a substitution, deletion, or addition of 1 to 9 amino acids other than the amino acids at positions 1, 4, 6, 9, and 34, and having a guanine-binding property;
(G-3) a PPR motif having a sequence identity of at least 80% to the sequence of (G-1), provided that the amino acids at positions 1, 4, 6, 9, and 34 are identical, and having a guanine-binding property;
(U-1) a PPR motif consisting of the sequence of SEQ ID NO: 10 having such substitutions of the amino acids at positions 6 and 9 that any one of the combinations defined below is satisfied;
(U-2) a PPR motif consisting of the sequence of (U-1) having a substitution, deletion, or addition of 1 to 9 amino acids other than the amino acids at positions 1, 4, 6, 9, and 34, and having a uracil-binding property; and
(U-3) a PPR motif having a sequence identity of at least 80% to the sequence of (U-1), provided that the amino acids at positions 1, 4, 6, 9, and 34 are identical, and having a uracil-binding property.
   a combination of asparagine as the amino acid at position 6 and glutamic acid as the amino acid at position 9,
   a combination of asparagine as the amino acid at position 6 and glutamine as the amino acid at position 9,
   a combination of asparagine as the amino acid at position 6 and lysine as the amino acid at position 9, and
   a combination of aspartic acid as the amino acid at position 6 and glycine as the amino acid at position 9.
[3] Use of the PPR motif according to 1 or 2 in a PPR protein as the first PPR motif from the N-terminus.
[4] The use according to 3, which is for reducing aggregation of the PPR protein.
[5] A protein capable of binding to a target nucleic acid having a specific nucleotide sequence, which comprises 1 to 30 of PPR motifs represented by the formula 1 mentioned below, and wherein the A₆ amino acid of the first PPR motif (M₁) from the N-terminus is a hydrophilic amino acid:

[Formula 4]

(Helix A)-X-(Helix B)-L (Formula 1)
wherein, in the formula:
   Helix A is a moiety of 12-amino acid length capable of forming an α-helix structure, and is represented by the formula 2;

[Formula 5]

A₁-A₂-A₃-A₄-A₅-A₆-A₇-A₈-A₉-A₁₀-A₁₁-A₁₂ (Formula 2)
wherein, in the formula 2, A₁ to A₁₂ independently represent an amino acid;
   X does not exist, or is a moiety of 1- to 9-amino acid length;
   Helix B is a moiety of 11- to 13-amino acid length capable of forming an α-helix structure; and
   L is a moiety of 2- to 7-amino acid length represented by the formula 3;

[Formula 6]

Lᵥᵢᵢ-Lᵥᵢ-Lᵥ-Lᵢᵥ-Lᵢᵢᵢ-Lᵢᵢ-Lᵢ (Formula 3)
wherein, in the formula 3, the amino acids are numbered “i” (-1), “ii” (-2), and so on from the C-terminus side,
   provided that Lᵢᵢᵢ to Lᵥᵢᵢ may not exist.
[6] The protein according to 5, wherein the A₉ amino acid of M₁ is a hydrophilic amino acid or glycine.
[7] The protein according to 5 or 6, wherein the A₆ amino acid of M₁ is asparagine or aspartic acid.
[8] The protein according to any one of 5 to 7, wherein the A₉ amino acid of M₁ is glutamine, glutamic acid, lysine, or glycine.
[9] The protein according to any one of 5 to 8, wherein the A₆ amino acid of M₁ and the A₉ amino acid of M₁ correspond to any of the following combinations:
   combination of asparagine as the A₆ amino acid and glutamic acid as the A₉ amino acid,
   combination of asparagine as the A₆ amino acid and glutamine as the A₉ amino acid,
   combination of asparagine as the A₆ amino acid and lysine as the A₉ amino acid, and
   combination of aspartic acid as the A₆ amino acid and glycine as the A₉ amino acid.
[10] A fusion protein of at least one selected from the group consisting of a fluorescent protein, a nuclear localization signal peptide, and a tag protein, and a PPR protein containing the PPR motif according to 1 or 2 as the first PPR motif from the N-terminus, or the protein according to any one of 5 to 9.
[11] A method for modifying a PPR protein containing the PPR motif according to 3 and capable of binding to a target nucleic acid having a specific nucleotide sequence, which comprises making the A₆ amino acid of the first PPR motif (M₁) from the N-terminus more hydrophilic.
[12] A method for detecting a nucleic acid, which uses a PPR protein containing the PPR motif according to 1 or 2 as the first PPR motif from the N-terminus, the protein according to any one of 5 to 9, or the fusion protein according to 10.
[13] A nucleic acid encoding the PPR motif according to 1 or 2, a PPR protein containing the PPR motif according to 1 or 2 as the first PPR motif from the N-terminus, or the protein according to any one of 5 to 9.
[14] A vector comprising the nucleic acid according to 13.
[15] A cell (except for human individual) containing the vector according to 14.
[16] A method for manipulating a nucleic acid, which uses the PPR motif according to 1 or 2, a PPR protein containing the PPR motif according to 1 or 2 as the first PPR motif from the N-terminus, the protein according to any one of 5 to 9, or the vector according to 14 (implementation in human individual is excluded).
[17] A method for producing an organism, which comprises the manipulation method according to 16.

### BRIEF DESCRIPTION OF THE DRAWINGS

FIGS. 1A and 1B The method for designing a PPR motif. FIG. 1A: The 6th and 9th amino acids of the first motif are exposed to the outside. FIG. 1B: For the 6th and 9th amino acids of the first motif that recognize cytosine, there were chosen leucine and glycine (C_6L9G) as a typical combination, and leucine and glutamic acid (C_6L9E), asparagine and glutamine (C_6N9Q), asparagine and glutamic acid (C_6N9E), asparagine and lysine (C_6N9K), and aspartic acid and glycine (C_6D9G) as mutant types.

FIG. 2 Aggregation and localization into nuclei of each PPR protein. Intracellular expression of PPR fused with GFP and a nuclear localization signal sequence was confirmed on the basis of fluorescence microscopy images. When the protein was fused with EGFP, it was observed that PPRcag_1 (6L9G) and PPRcag_2 (6L9E) did not localize to the nuclei, but strongly aggregated around the nuclei. On the other hand, PPRcag_3 (6N9Q), PPRcag_4 (6N9E), PPRcag_5 (6N9K), and PPRcag_6 (6D9G) did not localize to the nuclei, although their aggregation was weak. When mClover3 was fused, it was observed that PPRcag_1 (6L9G) and PPRcag_2 (6L9E) localized to the nuclei, but they aggregated in the nuclei. PPRcag_3 (6N9Q), PPRcag_4 (6N9E), PPRcag_5 (6N9K), and PPRcag_6 (6D9G) localized to the nuclei, and showed no aggregation.

FIG. 3 Binding experiment of PPR protein and RNA. It was found that all PPR proteins including amino acid mutations of the 6th and 9th amino acids specifically bind to the target CAGx6. In comparison with PPRcag_1, the binding power to the target sequence was substantially the same for PPRcag_2, about 80% for PPRcag_3, about 60% for PPRcag_4, about 120% for PPRcag_5, and about 130% for PPRcag_6.

FIG. 4 The effect of the first PPR motif from the N-terminus on aggregation. Each PPR protein was prepared in an E. coli expression system, purified, and separated by gel filtration chromatography. The smaller volume of the elution fraction (Elution vol.) means a larger molecular size. The proteins using v2 were eluted in 8 to 10 mL of elution fractions, whereas the peaks of the proteins using v3.2 were observed in elution fractions of 12 to 14 mL. These results suggested possibility that the proteins using v2 aggregated due to the larger protein size thereof, and it was found that the aggregation was improved in the proteins using v3.2.

### MODES FOR CARRYING OUT THE INVENTION

### [PPR Motif and PPR Protein]

### Definition

The PPR motif referred to in the present invention means a polypeptide constituted by 30 to 38 amino acids and having an amino acid sequence of an E value not larger than a predetermined value (desirably E-03) obtained for PF01535 in Pfam or PS51375 in Prosite as determined by amino acid sequence analysis with a protein domain search program on the Web, unless especially stated. The position numbers of amino acids constituting the PPR motif defined in the present invention are substantially synonymous with those of PF01535, and they correspond to those obtained by subtracting 2 from the numbers of the amino acid positions of PS51375 (for example, the position 1 referred to in the present invention corresponds to the position 3 of PS51375). Further, the term “ii” (-2)-th amino acid means the second amino acid from the end (C-terminus side) of the amino acids constituting the PPR motif, or the second amino acid towards the N-terminus side from the first amino acid of the following PPR motif, i.e., −2nd amino acid. When the following PPR motif is not definitely identified, the amino acid 2 amino acids before the first amino acid of the following helical structure is the amino acid of “ii”. For Pfam, http://pfam.sanger.ac.uk/ can be referred to, and for Prosite, http.//www.expasy.org/prosite/ can be referred to.

Although the conservativeness of the conserved amino acid sequence of the PPR motif is low at the amino acid level, two of the α-helixes as the secondary structure are well conserved. Although a typical PPR motif is constituted by 35 amino acids, the length thereof is as variable as is from 30 to 38 amino acids.

More specifically, the PPR motif referred to in the present invention consists of a polypeptide of a 30- to 38-amino acid length represented by the formula 1.

[Formula 7]

(Helix A)-X-(Helix B)-L (Formula 1)
wherein, in the formula:
   Helix A is a moiety of 12-amino acid length capable of forming an α-helix structure, and is represented by the formula 2;

[Formula 8]

A₁-A₂-A₃-A₄-A₅-A₆-A₇-A₈-A₉-A₁₀-A₁₁-A₁₂ (Formula 2)
wherein, in the formula 2, A₁ to A₁₂ independently represent an amino acid;
   X does not exist, or is a moiety of 1- to 9-amino acid length;
   Helix B is a moiety of 11- to 13-amino acid length capable of forming an α-helix structure; and
   L is a moiety of 2- to 7-amino acid length represented by the formula 3;

[Formula 9]

Lᵥᵢᵢ-Lᵥᵢ-Lᵥ-Lᵢᵥ-Lᵢᵢᵢ-Lᵢᵢ-Lᵢ (Formula 3)
wherein, in the formula 3, the amino acids are numbered “i” (-1), “ii” (-2), and so on from the C-terminus side,
   provided that Lᵢᵢᵢ to Lᵥᵢᵢ may not exist.

The term PPR protein used in the present invention refers to a PPR protein comprising one or more, preferably two or more, of the above-mentioned PPR motifs, unless especially indicated. The term protein used in this description refers to any substance consisting of a polypeptide (chain consisting of a plurality of amino acids bound via peptide bonds), unless especially indicated, and includes those consisting of a polypeptide of a comparatively low molecular weight. The term amino acid used in the present invention refers to a usual amino acid molecule, and also refers to an amino acid residue constituting a peptide chain. Which one is referred to shall be clear to those skilled in the art from the context.

In the present invention, the term specificity/specific used for the binding property of the PPR motif to a base in the target nucleic acid means that the binding activity to any one of the four bases is higher than the binding activities to the other bases, unless especially stated.

In the present invention, the term nucleic acid refers to RNA or DNA. Although the PPR protein may have specificity for bases in RNA or DNA, it does not bind to nucleic acid monomers.

In the PPR motif, combination of three of the 1st, 4th, and ii-th amino acids is important for specific binding to a base, and to which base the motif binds can be determined according to this combination (Patent document 1 and 2 mentioned above).

Specifically, with respect to the RNA-binding PPR motifs, the relationship between the combinations of three of the 1st, 4th, and ii-th amino acids and the bases to which they can bind is as follows (see Patent document 1 mentioned above).
(3-1) When the combination of the three amino acids of A₁, A₄, and Lᵢᵢ consists of valine, asparagine, and aspartic acid in this order, the PPR motif has such a selective RNA base-binding ability that the motif strongly binds to U, less strongly to C, and still less strongly to A or G.
(3-2) When the combination of the three amino acids of A₁, A₄, and Lᵢᵢ consists of valine, threonine, and asparagine in this order, the PPR motif has such a selective RNA base-binding ability that the motif strongly binds to A, less strongly to G, and still less strongly to C, but dose not bind to U.
(3-3) When the combination of the three amino acids of A₁, A₄, and Lᵢᵢ consists of valine, asparagine, and asparagine in this order, the PPR motif has such a selective RNA base-binding ability that the motif strongly binds to C, and less strongly to A or U, but does not bind to G.
(3-4) When the combination of the three amino acids of A₁, A₄, and Lᵢᵢ consists of glutamic acid, glycine, and aspartic acid in this order, the PPR motif has such a selective RNA base-binding ability that the motif strongly binds to G, but does not bind to A, U, and C.
(3-5) When the combination of the three amino acids of A₁, A₄, and Lᵢᵢ consists of isoleucine, asparagine, and asparagine in this order, the PPR motif has such a selective RNA base-binding ability that the motif strongly binds to C, less strongly to U, and still less strongly to A, but does not bind to G.
(3-6) When the combination of the three amino acids of A₁, A₄, and Lᵢᵢ consists of valine, threonine, and aspartic acid in this order, the PPR motif has such a selective RNA base-binding ability that the motif strongly binds to G, and less strongly to U, but does not bind to A and C.
(3-7) When the combination of the three amino acids of A₁, A₄, and Lᵢᵢ consists of lysine, threonine, and aspartic acid in this order, the PPR motif has such a selective RNA base-binding ability that the motif strongly binds to G, and less strongly to A, but does not bind to U and C.
(3-8) When the combination of the three amino acids of A₁, A₄, and Lᵢᵢ consists of phenylalanine, serine, and asparagine in this order, the PPR motif has such a selective RNA base-binding ability that the motif strongly binds to A, less strongly to C, and still less strongly to G and U.
(3-9) When the combination of the three amino acids of A₁, A₄, and Lᵢᵢ consists of valine, asparagine, and serine in this order, the PPR motif has such a selective RNA base-binding ability that the motif strongly binds to C, and less strongly to U, but does not bind to A and G.
(3-10) When the combination of the three amino acids of A₁, A₄, and Lᵢᵢ consists of phenylalanine, threonine, and asparagine in this order, the PPR motif has such a selective RNA base-binding ability that the motif strongly binds to A, but does not bind to G, U, and C.
(3-11) When the combination of the three amino acids of A₁, A₄, and Lᵢᵢ consists of isoleucine, asparagine, and aspartic acid in this order, the PPR motif has such a selective RNA base-binding ability that the motif strongly binds to U, and less strongly to A, but does not bind to G and C.
(3-12) When the combination of the three amino acids of A₁, A₄, and Lᵢᵢ consists of threonine, threonine, and asparagine in this order, the PPR motif has such a selective RNA base-binding ability that the motif strongly binds to A, but does not bind to G, U, and C.
(3-13) When the combination of the three amino acids of A₁, A₄, and Lᵢᵢ consists of isoleucine, methionine, and aspartic acid in this order, the PPR motif has such a selective RNA base-binding ability that the motif strongly binds to U, and less strongly to C, but does not bind to A and G.
(3-14) When the combination of the three amino acids of A₁, A₄, and Lᵢᵢ consists of phenylalanine, proline, and aspartic acid in this order, the PPR motif has such a selective RNA base-binding ability that the motif strongly binds to U, and less strongly to C, but does not bind to A and G.
(3-15) When the combination of the three amino acids of A₁, A₄, and Lᵢᵢ consists of tyrosine, proline, and aspartic acid in this order, the PPR motif has such a selective RNA base-binding ability that the motif strongly binds to U, but does not bind to A, G, and C.
(3-16) When the combination of the three amino acids of A₁, A₄, and Lᵢᵢ consists of leucine, threonine, and aspartic acid in this order, the PPR motif has such a selective RNA base-binding ability that the motif strongly binds to G, but does not bind to A, U, and C.

Specifically, with respect to the DNA-binding PPR motifs, the relationship between combinations of the three of the 1st, 4th, and ii-th amino acids and the bases to which they can bind is as follows (see Patent document 2 mentioned above).
(2-1) When the combination of the three amino acids of A₁, A₄, and Lᵢᵢ consists of an arbitrary amino acid, glycine, and aspartic acid in this order, the PPR motif selectively binds to G.
(2-2) When the combination of the three amino acids of A₁, A₄, and Lᵢᵢ consists of glutamic acid, glycine, and aspartic acid in this order, the PPR motif selectively binds to G.
(2-3) When the combination of the three amino acids of A₁, A₄, and Lᵢᵢ consists of an arbitrary amino acid, glycine, and asparagine in this order, the PPR motif selectively binds to A.
(2-4) When the combination of the three amino acids of A₁, A₄, and Lᵢᵢ consists of glutamic acid, glycine, and asparagine in this order, the PPR motif selectively binds to A.
(2-5) When the combination of the three amino acids of A₁, A₄, and Lᵢᵢ consists of an arbitrary amino acid, glycine, and serine in this order, the PPR motif selectively binds to A, and less selectively to C.
(2-6) When the combination of the three amino acids of A₁, A₄, and Lᵢᵢ consists of an arbitrary amino acid, isoleucine, and an arbitrary amino acid in this order, the PPR motif selectively binds to T and C.
(2-7) When the combination of the three amino acids of A₁, A₄, and Lᵢᵢ consists of an arbitrary amino acid, isoleucine, and asparagine in this order, the PPR motif selectively binds to T, and less selectively to C.
(2-8) When the combination of the three amino acids of A₁, A₄, and Lᵢᵢ consists of an arbitrary amino acid, leucine, and an arbitrary amino acid in this order, the PPR motif selectively binds to T and C.
(2-9) When the combination of the three amino acids of A₁, A₄, and Lᵢᵢ consists of an arbitrary amino acid, leucine, and aspartic acid in this order, the PPR motif selectively binds to C.
(2-10) When the combination of the three amino acids of A₁, A₄, and Lᵢᵢ consists of an arbitrary amino acid, leucine, and lysine in this order, the PPR motif selectively binds to T.
(2-11) When the combination of the three amino acids of A₁, A₄, and Lᵢᵢ consists of an arbitrary amino acid, methionine, and an arbitrary amino acid in this order, the PPR motif selectively binds to T.
(2-12) When the combination of the three amino acids of A₁, A₄, and Lᵢᵢ consists of an arbitrary amino acid, methionine, and aspartic acid in this order, the PPR motif selectively binds to T.
(2-13) When the combination of the three amino acids of A₁, A₄, and Lᵢᵢ consists of isoleucine, methionine, and aspartic acid in this order, the PPR motif selectively binds to T, and less selectively to C.
(2-14) When the combination of the three amino acids of A₁, A₄, and Lᵢᵢ consists of an arbitrary amino acid, asparagine, and an arbitrary amino acid in this order, the PPR motif selectively binds to C and T.
(2-15) When the combination of the three amino acids of A₁, A₄, and Lᵢᵢ consists of an arbitrary amino acid, asparagine, and aspartic acid in this order, the PPR motif selectively binds to T.
(2-16) When the combination of the three amino acids of A₁, A₄, and Lᵢᵢ consists of phenylalanine, asparagine, and aspartic acid in this order, the PPR motif selectively binds to T.
(2-17) When the combination of the three amino acids of A₁, A₄, and Lᵢᵢ consists of glycine, asparagine, and aspartic acid in this order, the PPR motif selectively binds to T.
(2-18) When the combination of the three amino acids of A₁, A₄, and Lᵢᵢ consists of isoleucine, asparagine, and aspartic acid in this order, the PPR motif selectively binds to T.
(2-19) When the combination of the three amino acids of A₁, A₄, and Lᵢᵢ consists of threonine, asparagine, and aspartic acid in this order, the PPR motif selectively binds to T.
(2-20) When the combination of the three amino acids of A₁, A₄, and Lᵢᵢ consists of valine, asparagine, and aspartic acid in this order, the PPR motif selectively binds to T, and less selectively to C.
(2-21) When the combination of the three amino acids of A₁, A₄, and Lᵢᵢ consists of tyrosine, asparagine, and aspartic acid in this order, the PPR motif selectively binds to T, and less selectively to C.
(2-22) When the combination of the three amino acids of A₁, A₄, and Lᵢᵢ consists of an arbitrary amino acid, asparagine, and asparagine in this order, the PPR motif selectively binds to C.
(2-23) When the combination of the three amino acids of A₁, A₄, and Lᵢᵢ consists of isoleucine, asparagine, and asparagine in this order, the PPR motif selectively binds to C.
(2-24) When the combination of the three amino acids of A₁, A₄, and Lᵢᵢ consists of serine, asparagine, and asparagine in this order, the PPR motif selectively binds to C.
(2-25) When the combination of the three amino acids of A₁, A₄, and Lᵢᵢ consists of valine, asparagine, and asparagine in this order, the PPR motif selectively binds to C.
(2-26) When the combination of the three amino acids of A₁, A₄, and Lᵢᵢ consists of an arbitrary amino acid, asparagine, and serine in this order, the PPR motif selectively binds to C.
(2-27) When the combination of the three amino acids of A₁, A₄, and Lᵢᵢ consists of valine, asparagine, and serine in this order, the PPR motif selectively binds to C.
(2-28) When the combination of the three amino acids of A₁, A₄, and Lᵢᵢ consists of an arbitrary amino acid, asparagine, and threonine in this order, the PPR motif selectively binds to C.
(2-29) When the combination of the three amino acids of A₁, A₄, and Lᵢᵢ consists of valine, asparagine, and threonine in this order, the PPR motif selectively binds to C.
(2-30) When the combination of the three amino acids of A₁, A₄, and Lᵢᵢ consists of an arbitrary amino acid, asparagine, and tryptophan in this order, the PPR motif selectively binds to C, and less selectively to T.
(2-31) When the combination of the three amino acids of A₁, A₄, and Lᵢᵢ consists of isoleucine, asparagine, and tryptophan in this order, the PPR motif selectively binds to T, and less selectively to C.
(2-32) When the combination of the three amino acids of A₁, A₄, and Lᵢᵢ consists of an arbitrary amino acid, proline, and an arbitrary amino acid in this order, the PPR motif selectively binds to T.
(2-33) When the combination of the three amino acids of A₁, A₄, and Lᵢᵢ consists of an arbitrary amino acid, proline, and aspartic acid in this order, the PPR motif selectively binds to T.
(2-34) When the combination of the three amino acids of A₁, A₄, and Lᵢᵢ consists of phenylalanine, proline, and aspartic acid in this order, the PPR motif selectively binds to T.
(2-35) When the combination of the three amino acids of A₁, A₄, and Lᵢᵢ consists of tyrosine, proline, and aspartic acid in this order, the PPR motif selectively binds to T.
(2-36) When the combination of the three amino acids of A₁, A₄, and Lᵢᵢ consists of an arbitrary amino acid, serine, and an arbitrary amino acid in this order, the PPR motif selectively binds to A and G.
(2-37) When the combination of the three amino acids of A₁, A₄, and Lᵢᵢ consists of an arbitrary amino acid, serine, and asparagine in this order, the PPR motif selectively binds to A.
(2-38) When the combination of the three amino acids of A₁, A₄, and Lᵢᵢ consists of phenylalanine, serine, and asparagine in this order, the PPR motif selectively binds to A.
(2-39) When the combination of the three amino acids of A₁, A₄, and Lᵢᵢ consists of valine, serine, and asparagine in this order, the PPR motif selectively binds to A.
(2-40) When the combination of the three amino acids of A₁, A₄, and Lᵢᵢ consists of an arbitrary amino acid, threonine, and an arbitrary amino acid in this order, the PPR motif selectively binds to A and G.
(2-41) When the combination of the three amino acids of A₁, A₄, and Lᵢᵢ consists of an arbitrary amino acid, threonine, and aspartic acid in this order, the PPR motif selectively binds to G.
(2-42) When the combination of the three amino acids of A₁, A₄, and Lᵢᵢ consists of valine, threonine, and aspartic acid in this order, the PPR motif selectively binds to G.
(2-43) When the combination of the three amino acids of A₁, A₄, and Lᵢᵢ consists of an arbitrary amino acid, threonine, and asparagine in this order, the PPR motif selectively binds to A.
(2-44) When the combination of the three amino acids of A₁, A₄, and Lᵢᵢ consists of phenylalanine, threonine, and asparagine in this order, the PPR motif selectively binds to A.
(2-45) When the combination of the three amino acids of A₁, A₄, and Lᵢᵢ consists of isoleucine, threonine, and asparagine in this order, the PPR motif selectively binds to A.
(2-46) When the combination of the three amino acids of A₁, A₄, and Lᵢᵢ consists of valine, threonine, and asparagine in this order, the PPR motif selectively binds to A.
(2-47) When the combination of the three amino acids of A₁, A₄, and Lᵢᵢ consists of an arbitrary amino acid, valine, and an arbitrary amino acid in this order, the PPR motif binds to A, C, and T, but does not bind to G.
(2-48) When the combination of the three amino acids of A₁, A₄, and Lᵢᵢ consists of isoleucine, valine, and aspartic acid in this order, the PPR motif selectively binds to C, and less selectively to A.
(2-49) When the combination of the three amino acids of A₁, A₄, and Lᵢᵢ consists of an arbitrary amino acid, valine, and glycine in this order, the PPR motif selectively binds to C.
(2-50) When the combination of the three amino acids of A₁, A₄, and Lᵢᵢ consists of an arbitrary amino acid, valine, and threonine in this order, the PPR motif selectively binds to T.

### (Particularly Preferred Combinations of the Three Amino Acids)

For the RNA-binding PPR motifs, there are typical combinations of the 1st, 4th, and ii-th amino acids that can recognize and specifically bind to each base. Specifically, the combination that recognizes adenine consists of 1st valine, 4th threonine, and ii-th asparagine; the combination that recognizes cytosine consists of 1st valine, 4th asparagine, and ii-th serine; the combination that recognizes guanine consists of 1st valine, 4th threonine, and ii-th aspartic acid, and the combination that recognizes uracil consists of 1st is valine, 4th is asparagine, and ii-th aspartic acid (Non-patent documents 1 to 5 mentioned above). In one of the preferred embodiments of the present invention, these combinations are used.

### (Improvement of Aggregation Property)

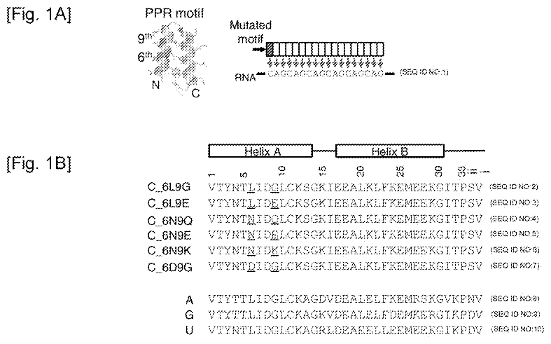

The inventors of the present invention found that the amino acid at position 6 of the PPR motif is extremely frequently hydrophobic amino acid (especially leucine) and the amino acid at position 9 is extremely frequently a non-hydrophilic amino acid (especially glycine) on the basis of the amino acid information of existing naturally occurring PPR motifs. On the basis of structures of the PPR proteins for which crystal structures have already been obtained (Non-patent document 6: Coquille et al., 2014 Nat. Commun., PDB ID: 4PJQ, 4WN4, 4WSL, 4PJR, Non-patent document 7: Shen et al., 2015 Nat. Commun., PDB ID: 519D, 5I9F, 5I9G, 5I9H), they imagined that since those 6th and 9th amino acids in the first motif (N-terminus side) are exposed to the outside, the proteins show aggregation property due to these exposed hydrophobic amino acids (FIG. 1A). On the other hand, they considered that, in the second and following motifs, the 6th and 9th amino acids are buried inside the protein, and form a hydrophobic core, and therefore if hydrophilic residues are placed as the 6th and 9th amino acids of all the motifs, the protein structure may collapse. Therefore, they decided to decrease the aggregation property of PPR by using hydrophilic amino acid (asparagine, aspartic acid, glutamine, glutamic acid, lysine, arginine, serine, and threonine) as the 6th amino acid, preferably the 6th and 9th amino acids, in only the first motif.

Specific procedure is as follows.

In the first PPR motif (M₁) from the N-terminus of a protein capable of binding to a target nucleic acid having a specific nucleotide sequence:
(1) a hydrophilic amino acid is used as the A₆ amino acid, preferably asparagine or aspartic acid is used as the A₆ amino acid,
(2) further, a hydrophilic amino acid or glycine, preferably glutamine, glutamic acid, lysine, or glycine, is used as the A₉ amino acid, or
(3) the A₆ amino acid and A₉ amino acid are constituted by any of the following combinations;
   combination of asparagine as the A₆ amino acid and glutamic acid as the A₉ amino acid,
   combination of asparagine as the A₆ amino acid and glutamine as the A₉ amino acid,
   combination of asparagine as the A₆ amino acid and lysine as the A₉ amino acid, and
   combination of aspartic acid as the A₆ amino acid and glycine as the A₉ amino acid.

### (Novel PPR Motif)

The present invention provides novel PPR motifs with improved aggregation property and novel PPR proteins containing the same, which were found as described above.

The novel PPR motifs provided by the present invention are followings:
(C-1) a PPR motif consisting of any one of the sequences of SEQ ID NOS: 4 to 7;
(C-2) a PPR motif consisting of any one of the sequences of SEQ ID NOS: 4 to 7 having a substitution, deletion, or addition of 1 to 9 amino acids other than the amino acids at positions 1, 4, 6, and 34, and having a cytosine-binding property;
(C-3) a PPR motif having a sequence identity of at least 80% to any one of the sequences of SEQ ID NOS: 4 to 7, provided that the amino acids at positions 1, 4, 6, and 34 are identical, and having a cytosine-binding property;
(A-1) a PPR motif consisting of the sequence of SEQ ID NO: 8 having a substitution of the amino acid at position 6 with asparagine or aspartic acid;
(A-2) a PPR motif consisting of the sequence of (A-1) having a substitution, deletion, or addition of 1 to 9 amino acids other than the amino acids at positions 1, 4, 6, and 34, and having an adenine-binding property;
(A-3) a PPR motif having a sequence identity of at least 80% to the sequence of (A-1), provided that the amino acids at positions 1, 4, 6, and 34 are identical, and having an adenine-binding property;
(G-1) a PPR motif consisting of the sequence of SEQ ID NO: 9 having a substitution of the amino acid at position 6 with asparagine or aspartic acid;
(G-2) a PPR motif consisting of the sequence of (G-1) having a substitution, deletion, or addition of 1 to 9 amino acids other than the amino acids at positions 1, 4, 6, and 34, and having a guanine-binding property;
(G-3) a PPR motif having a sequence identity of at least 80% to the sequence of (G-1), provided that the amino acids at positions 1, 4, 6, and 34 are identical, and having a guanine-binding property;
(U-1) a PPR motif consisting of the sequence of SEQ ID NO: 10 having a substitution of the amino acid at position 6 with asparagine or aspartic acid;
(U-2) a PPR motif consisting of the sequence of (U-1) having a substitution, deletion, or addition of 1 to 9 amino acids other than the amino acids at positions 1, 4, 6, and 34, and having a uracil-binding property; and
(U-3) a PPR motif having a sequence identity of at least 80% to the sequence of (U-1), provided that the amino acids at positions 1, 4, 6, and 34 are identical, and having a uracil-binding property.

Among such PPR motifs, the followings are particularly preferred:
(C-1) a PPR motif consisting of any one of the sequences of SEQ ID NOS: 4 to 7;
(C-2) a PPR motif consisting of any one of the sequences of SEQ ID NOS: 4 to 7 having a substitution, deletion, or addition of 1 to 9 amino acids other than the amino acids at positions 1, 4, 6, 9, and 34, and having a cytosine-binding property;
(C-3) a PPR motif having a sequence identity of at least 80% to any one of the sequences of SEQ ID NOS: 4 to 7, provided that the amino acids at positions 1, 4, 6, 9, and 34 are identical, and having a cytosine-binding property;
(A-1) a PPR motif consisting of the sequence of SEQ ID NO: 8 having such substitutions of the amino acids at positions 6 and 9 that any one of the combinations defined below is satisfied;
(A-2) a PPR motif consisting of the sequence of (A-1) having a substitution, deletion, or addition of 1 to 9 amino acids other than the amino acids at positions 1, 4, 6, 9, and 34, and having an adenine-binding property;
(A-3) a PPR motif having a sequence identity of at least 80% to the sequence of (A-1), provided that the amino acids at positions 1, 4, 6, 9, and 34 are identical, and having an adenine-binding property;
(G-1) a PPR motif consisting of the sequence of SEQ ID NO: 9 having such substitutions of the amino acids at positions 6 and 9 that any one of the combinations defined below is satisfied;
(G-2) a PPR motif consisting of the sequence of (G-1) having a substitution, deletion, or addition of 1 to 9 amino acids other than the amino acids at positions 1, 4, 6, 9, and 34, and having a guanine-binding property;
(G-3) a PPR motif having a sequence identity of at least 80% to the sequence of (G-1), provided that the amino acids at positions 1, 4, 6, 9, and 34 are identical, and having a guanine-binding property;
(U-1) a PPR motif consisting of the sequence of SEQ ID NO: 10 having such substitutions of the amino acids at positions 6 and 9 that any one of the combinations defined below is satisfied;
(U-2) a PPR motif consisting of the sequence of (U-1) having a substitution, deletion, or addition of 1 to 9 amino acids other than the amino acids at positions 1, 4, 6, 9, and 34, and having a uracil-binding property;
(U-3) a PPR motif having a sequence identity of at least 80% to the sequence of (U-1), provided that the amino acids at positions 1, 4, 6, 9, and 34 are identical, and having a uracil-binding property:
   a combination of asparagine as the amino acid at position 6 and glutamic acid as the amino acid at position 9,
   a combination of asparagine as the amino acid at position 6 and glutamine as the amino acid at position 9,
   a combination of asparagine as the amino acid at position 6 and lysine as the amino acid at position 9, and
   a combination of aspartic acid as the amino acid at position 6 and glycine as the amino acid at position 9.

The specific sequences of SEQ ID NOS: 4 to 10 are shown in FIGS. 1A and 1B, and in the sequence listing.

Among such PPR motifs, more preferred are the followings:
(C-4) a PPR motif consisting of the sequence of SEQ ID NO: 4;
(A-4) a PPR motif consisting of the sequence of SEQ ID NO: 58;
(G-4) a PPR motif consisting of the sequence of SEQ ID NO: 59; and
(U-4) a PPR motif consisting of the sequence of SEQ ID NO: 60.

The sequences of SEQ ID NOS: 58 to 60 are shown below and in the

### SEQUENCE LISTING

Sequence of SEQ ID NO: 58:
VTYTTNIDQLCKAGKVDEALELFKEMRSKGVKPNV
Sequence of SEQ ID NO: 59:
VTYTTNIDQLCKAGKVDEALELFDEMKERGIKPDV
Sequence of SEQ ID NO: 60:
VTYNTNIDQLCKAGRLDEAEELLEEMEEKGIKPDV

(PPR Protein with Improved Aggregation Property)

The present invention also provides PPR proteins with improved aggregation properties found as described above.

In one of the preferred embodiments, the A₉ amino acid of M₁ is a non-hydrophobic amino acid or glycine, whatever the other amino acids of M₁ are, and whatever the amino acid sequences of the motifs other than M₁ are. The non-hydrophobic amino acid is a hydrophilic amino acid, or cysteine or histidine; preferably a hydrophilic amino acid, i.e., arginine, asparagine, aspartic acid, glutamic acid, glutamine, lysine, serine, or threonine; more preferably glutamine, glutamic acid, or lysine.

In one of the preferred embodiments, the A₉ amino acid of M₁ is glutamine, glutamic acid, lysine, or glycine, whatever the other amino acids of M₁ are, and whatever the amino acid sequences of the motifs other than M₁ are.

In one of the preferred embodiments, the A₆ amino acid of M₁ is a non-hydrophobic amino acid, whatever the other amino acids of M₁ are, and whatever the amino acid sequences of the motifs other than M₁ are. The non-hydrophobic amino acid is, for example, a hydrophilic amino acid, or cysteine or histidine; preferably a hydrophilic amino acid, i.e., arginine, asparagine, aspartic acid, glutamic acid, glutamine, lysine, serine, or threonine; more preferably asparagine, or aspartic acid.

In one of the particularly preferred embodiments, the A₆ and A₉ amino acids of M₁ consist of any of the following combination, whatever the other amino acids of M₁ are, and whatever the amino acid sequences of the motifs other than M₁ are:
combination of asparagine as the A₆ amino acid and glutamic acid as the A₉ amino acid,
combination of asparagine as the A₆ amino acid and glutamine as the A₉ amino acid,
combination of asparagine as the A₆ amino acid and lysine as the A₉ amino acid, and
combination of aspartic acid as the A₆ amino acid and glycine as the A₉ amino acid.

In one of the preferred embodiments of the RNA-binding protein, the A₆ and A₉ amino acids of M₁ satisfy the above conditions, and at least one, preferably half or more, more preferably all, of the included PPR motifs satisfy any of the following conditions:
when the base to be bound is cytosine, A₁ is valine, A₄ is asparagine, and Aᵢᵢ is serine;
when the base to be bound is adenine, A₁ is valine, A₄ is threonine, and Aᵢᵢ is asparagine;
when the base to be bonded is guanine, A₁ is valine, A₄ is threonine, and Aᵢᵢ is aspartic acid; and
when the base to be bound is uracil or thymine, A₁ is valine, A₄ is asparagine, and Aᵢᵢ is aspartic acid.

In one of the preferred embodiments of the RNA-binding protein, M₁ is the novel PPR motif described above.

In one of the particularly preferred embodiments, M₁ is a PPR motif consisting of any of the following polypeptides:
a polypeptide consisting of any one of the sequences of SEQ ID NOS: 4 to 7 for cytosine as the base to be bound;
a polypeptide consisting of the sequence of SEQ ID NO: 8 having such substitutions of the amino acids at positions 6 and 9 that any one of the combinations defined in the following paragraph is satisfied for adenine as the base to be bound;
a polypeptide consisting of the sequence of SEQ ID NO: 9 having such substitutions of the amino acids at positions 6 and 9 that any one of the combinations defined in the following paragraph is satisfied for guanine as the base to be bound; and
a polypeptide consisting of the sequence of SEQ ID NO: 10 having such substitutions of the amino acids at positions 6 and 9 that any one of the combinations defined in the following paragraph is satisfied for uracil as the base to be bound;

At least one of the PPR motifs other than M₁ is a PPR motif consisting of any one of the following polypeptides:
a polypeptide consisting of the sequence of SEQ ID NO: 2 for cytosine as the base to be bound;
a polypeptide consisting of the sequence of SEQ ID NO: 8 for adenine as the base to be bound;
a polypeptide consisting of the sequence of SEQ ID NO: 9 for guanine as the base to be bound; and
a polypeptide consisting of the sequence of SEQ ID NO: 10 for uracil as the base to be bound.

The combinations referred to in the above paragraph are any of the followings:
a combination of asparagine as the A₆ amino acid and glutamic acid as the A₉ amino acid at position 9,
a combination of asparagine as the A₆ amino acid and glutamine as the A₉ amino acid,
a combination of asparagine as the A₆ amino acid and lysine as the A₉ amino acid, and
a combination of aspartic acid as the A₆ amino acid and glycine as the A₉ amino acid.

In one of the particularly preferred embodiments, M₁ is a PPR motif consisting of any one of the following polypeptides:
a polypeptide consisting of the sequence of SEQ ID NO: 4 for cytosine as the base to be bound;
a polypeptide consisting of the sequence of SEQ ID NO: 58 for adenine as the base to be bound;
a polypeptide consisting of the sequence of SEQ ID NO: 59 for guanine as the base to be bound; and
a polypeptide consisting of the sequence of SEQ ID NO: 60 for uracil as the base to be bound.

At least one of the PPR motifs other than M₁ is a PPR motif consisting of any one of the following polypeptides:
a polypeptide consisting of the sequence of SEQ ID NO: 2 for cytosine as the base to be bound;
a polypeptide consisting of the sequence of SEQ ID NO: 8 having a substitution of the amino acid at position 15 with lysine for adenine as the base to be bound;
a polypeptide consisting of the sequence of SEQ ID NO: 9 for guanine as the base to be bound; and
a polypeptide consisting of the sequence of SEQ ID NO: 10 for uracil as the base to be bound.

### (Use of Skeleton of High Performance PPR Motif)

In one of the preferred embodiments of the present invention, the amino acids in the PPR motifs for cytosine, adenine, guanine, and uracil (or thymine) other than the amino acids at positions 1, 4, 6, 9 and ii can be particular amino acids. Precisely, from the Arabidopsis thaliana PPR motif sequences, there were collected the PPR motifs in which the combination of amino acids at positions 1, 4, and ii is VTN as adenine-recognizing PPR motifs, those in which the same is VSN as the cytosine-recognizing PPR motifs, those in which the same is VTD as the guanine-recognizing PPR motifs, and those in which the same is VND as the uracil-recognizing PPR motifs, and types and occurring numbers of the amino acids at every position are summarized. Then, by selecting amino acid highly frequently occurring at each position, the performance of the PPR motif can be enhanced.

For the purpose of using highly frequently occurring amino acids as the amino acids other than the 1st, 4th, 6th, 9th and ii-th amino acids as described above, for obtaining an RNA-binding PPR protein, the amino acid sequences of the following PPR motifs can be referred to:
a PPR motif consisting of any one of the sequences of SEQ ID NOS: 4 to 7 as a PPR motif for cytosine;
a PPR motif consisting of the sequence of SEQ ID NO: 8 as the PPR motif for adenine;
a PPR motif consisting of the sequence of SEQ ID NO: 9 as the PPR motif for guanine; and
a PPR motif consisting of the sequence of SEQ ID NO: 10 as the PPR motif for guanine;

### Explanation of Technical Terms, Etc

The term “identity” used in the present invention for base sequence (also referred to as nucleotide sequence) or amino acid sequence means percentage of number of matched bases or amino acids shared between two sequences aligned in an optimal manner, unless especially stated. In other words, the identity can be calculated in accordance with the equation: Identity=(Number of matched positions/Total number of positions)×100, and it can be calculated by using commercially available algorithms. Such algorithms are also incorporated in the NBLAST and XBLAST programs described in Altschul et al., J. Mol. Biol., 215 (1990) 403-410. In more detail, the search and analysis for the identity of nucleotide or amino acid sequences can be performed with algorithms or programs well known to those skilled in the art (e.g., BLASTN, BLASTP, BLASTX, and ClustalW). In the case of using a program, parameters can be appropriately set by those skilled in the art, or the default parameters of each program can also be used. The specific procedures of these analysis methods are also well known to those skilled in the art.

In this description, when the expression of having an identity (or identity is high) is used for a nucleotide sequence or amino acid sequence, it means for both cases to have an identity of, at least 70%, preferably 80% or higher, more preferably 85% or higher, still more preferably 90% or higher, further preferably 95% or higher, still further preferably 97.5% or higher, even more preferably 99% or higher, unless especially stated.

As for the term “sequence having a substitution, deletion, or addition” used in the present invention concerning PPR motif or protein, the number of amino acids substituted or the like is not particularly limited in any motif or protein, so long as the motif or protein consisting of the amino acid sequence has the desired function, unless especially stated. The number of amino acids to be substituted, or the like may be about 1 to 9 or 1 to 4, or even larger number of amino acids may be substituted or the like if they are substituted with amino acids having similar properties. The means for preparing polynucleotides or proteins for such amino acid sequences are well known to those skilled in the art.

Amino acids having similar properties refer to amino acids with similar physical properties such as hydropathy, charge, pKa, and solubility, and refer to such amino acid as mentioned below, for example.
Hydrophobic amino acids; alanine, valine, glycine, isoleucine, leucine, phenylalanine, proline, tryptophan, tyrosine.
Non-hydrophobic amino acids; arginine, asparagine, aspartic acid, glutamic acid, glutamine, lysine, serine, threonine, cysteine, histidine, methionine.
Hydrophilic amino acids; arginine, asparagine, aspartic acid, glutamic acid, glutamine, lysine, serine, threonine.
Acidic amino acids: aspartic acid, glutamic acid.
Basic amino acids: lysine, arginine, histidine.
Neutral amino acids: alanine, asparagine, cysteine, glutamine, glycine, isoleucine, leucine, methionine, phenylalanine, proline, serine, threonine, tryptophan, tyrosine, valine.
Sulfur-containing amino acids: methionine, cysteine.
Aromatic ring-containing amino acids: tyrosine, tryptophan, phenylalanine.

The term “preparation” used for genes, nucleic acids, polynucleotides, proteins, motifs, etc. can be rephrased as “production” or “manufacturing”. In addition, the term “construction” is sometimes used to refer to preparation of genes or the like by combining parts, and “construction” can also be rephrased as “production” or “manufacturing”.

The PPR motif, protein containing the same, or nucleic acids encoding the same of the present invention can be prepared by those skilled in the art using conventional techniques, and the explanations in the section of Examples of this description.

### [Characteristics and Use of PPR Proteins]

### (Improvement of Aggregation Property of PPR Proteins)

The PPR proteins produced by using the novel PPR motifs of the present invention show reduced aggregation in cells. Aggregation of a PPR protein can be evaluated by those skilled in the art by expressing the PPR protein in cells and confirming presence or absence of aggregation. The confirmation is more easily performed by fusing the PPR protein to a fluorescent protein and expressing them. According to the examination of the inventors of the present invention, by appropriately modifying the amino acids in the 1st motif of the PPR protein, the aggregation property of the PPR protein in the cells is improved, and localization of the same to the nuclei is improved.

### (Binding Power)

PPR proteins prepared by using the novel PPR motifs of the present invention may have not only reduced intracellular aggregation property, but also RNA binding performance equivalent to or higher than those of PPR proteins for the same target RNA prepared by using existing PPR motifs. Equivalent means to be 55% or higher, preferably about 75%.

The binding power to a target sequence can be evaluated by EMSA (Electrophoretic Mobility Shift Assay) or a method using Biacore. EMSA is a method utilizing a property of nucleic acid that when a sample consisting of a nucleic acid bound with a protein is electrophoresed, the mobility of the nucleic acid molecule changes from that of the nucleic acid not bound. Molecular interaction analyzers, such as Biacore as a typical example, enable kinetic analysis, and therefore allow detailed protein-nucleic acid binding analysis.

The binding power to a target sequence can also be evaluated by adding a solution containing a candidate protein to a solid-phased target nucleic acid, and detecting or quantifying the protein that bound to the target nucleic acid. This method is sometimes referred to as the RPB-ELISA (RNA-protein binding ELISA) method, since it is utilizes ELISA (Enzyme-Linked Immuno Sorbent Assay). The step of adding a solution containing a candidate protein to a solid-phased target nucleic acid can be specifically carried out by flowing a solution containing the objective binding protein on the target nucleic acid molecule immobilized on a plate. Immobilization of the target nucleic acid molecule can be achieved by using various existing immobilization methods, such as by providing a nucleic acid probe containing a biotin-modified target nucleic acid molecule to a streptavidin-coated well plate. For detailed conditions of the experiments, the experiment methods descried in detail in the section of Examples in this description can be referred to. In RPB-ELISA, a value obtained by subtracting background signal (luminescence signal value obtained with an objective PPR protein without adding the target RNA) from luminescence obtained with a sample containing the objective PPR protein and the target RNA thereof can be used as the binding power of the objective PPR protein and the target RNA thereof.

### [Use of PPR Protein]

### (Complex and Fusion Protein)

The PPR motif or PPR protein provided by the present invention can be made into a complex by binding a functional region. The PPR motif or PPR protein can also be linked with a proteinaceous functional region to form a fusion protein. The functional region refers to a part having such a function as a specific biological function exerted in a living body or cell, for example, enzymatic function, catalytic function, inhibitory function, promotion function, etc, or a function as a marker. Such a region consists of, for example, a protein, peptide, nucleic acid, physiologically active substance, or drug. In the following explanations, the complex of the present invention may be explained with reference to a fusion protein as an example, but those skilled in the art may also understand complexes other than fusion protein according to the explanations.

In one of the preferred embodiments, the functional region is a ribonuclease (RNase). Examples of RNase are RNase A (e.g., bovine pancreatic ribonuclease A, PDB 2AAS), and RNase H.

In one of the preferred embodiments, the functional region is a fluorescent protein. Examples of fluorescent protein are mCherry, EGFP, GFP, Sirius, EBFP, ECFP, mTurquoise, TagCFP, AmCyan, mTFP1, MidoriishiCyan, CFP, TurboGFP, AcGFP, TagGFP, Azami-Green, ZsGreen, EmGFP, HyPer, TagYFP, EYFP, Venus, YFP, PhiYFP, PhiYFP-m, TurboYFP, ZsYellow, mBanana, KusabiraOrange, mOrange, TurboRFP, DsRed-Express, DsRed2, TagRFP, DsRed-Monomer, AsRed2, mStrawberry, TurboFP602, mRFP1, JRed, KillerRed, HcRed, KeimaRed, mRasberry, mPlum, PS-CFP, Dendra2, Kaede, EosFP, and KikumeGR. A preferred example is mClover3 in view of improvement of aggregation and/or efficient localization to the nuclei as a fusion protein.

In one preferred embodiment, when the target is mRNA, the functional region is a functional domain that enhances expression amount of a protein from the target mRNA (WO2017/209122). The functional domain that enhances expression amount of a protein from mRNA may be, for example, all or a functional part of a functional domain of a protein known to directly or indirectly promote translation of mRNA. More specifically, it may be a domain that directs ribosomes to mRNA, domain associated with initiating or promoting translation of mRNA, domain associated with transporting mRNA out of the nucleus, domain associated with binding to the endoplasmic reticulum membrane, domain containing an endoplasmic reticulum (ER) retention signal sequence, or domain containing an endoplasmic reticulum signal sequence. More specifically, the domain that directs ribosomes to mRNA mentioned above may be a domain comprising all or a functional part of a polypeptide selected from the group consisting of density-regulated protein (DENR), malignant T-cell amplified sequence 1 (MCT-1), transcriptionally-controlled tumor protein (TPT1), and Lerepo4 (zinc finger CCCH-domain). The domain associated with translation initiation or translation promotion of mRNA mentioned above may be a domain comprising all or a functional part of a polypeptide selected from the group consisting of eIF4E and eIF4G. The domain associated with transporting mRNA out of the nucleus mentioned above may be a domain containing all or a functional part of stem-loop binding protein (SLBP). The domain associated with binding to the endoplasmic reticulum membrane mentioned above may be a domain comprising all or a functional part of a polypeptide selected from the group consisting of SEC61B, translocation associated protein alpha (TRAP-alpha), SR-alpha, Dial (cytochrome b5 reductase 3), and p180. The endoplasmic reticulum retention signal (ER retention signal) sequence mentioned above may be a signal sequence comprising the KDEL (SEQ ID NO: 55) or KEEL (SEQ ID NO: 56) sequence. The endoplasmic reticulum signal sequence mentioned above may be a signal sequence including MGWSCIILFLVATATGAHS (SEQ ID NO: 57).

In the present invention, the functional region may be fused to the PPR protein on the N-terminal side or the C-terminal side, or on both the N-terminal side and the C-terminal side. The complex or fusion protein may include a plurality of functional regions (e.g., 2 to 5). Further, the complex or fusion protein according to the present invention may consist of the functional region and PPR protein indirectly fused via a linker or the like.

### (Nucleic Acid Encoding PPR Protein Etc., Vector, and Cell)

The present invention also provides a nucleic acid encoding the PPR motif, PPR protein or fusion protein mentioned above, and a vector containing such a nucleic acid (e.g., vector for amplification, and expression vector). As the host of the vector for amplification, E. coli or yeast may be used. In this description, expression vector means a vector containing, for example, a DNA having a promoter sequence, DNA encoding a desired protein, and DNA having a terminator sequence from the upstream side, but they need not necessarily be arranged in this order, so long as the desired function is exerted. In the present invention, recombinant vectors prepared by using various vectors that may be normally used by those skilled in the art may be used.

The PPR protein or fusion protein of the present invention can function in eukaryotic (e.g., animal, plant, microbe (yeast, etc.), and protozoan) cells. The fusion protein of the present invention can function, in particular, in animal cells (in vitro or in vivo). Examples of animal cells into which the PPR protein or fusion protein of the present invention, or a vector expressing it can be introduced include, for example, cells derived from humans, monkeys, pigs, cows, horses, dogs, cats, mice, and rats. Examples of cultured cells into which the PPR protein or fusion protein of the present invention or a vector expressing it can be introduced include, for example, Chinese hamster ovary (CHO) cells, COS-1 cells, COS-7 cells, VERO (ATCC CCL-81) cells, BHK cells, canine kidney-derived MDCK cells, hamster AV-12-664 cells, HeLa cells, W138 cells, 293 cells, 293T cells, and PER.C6 cells, but not limited to these.

### (Use)

With the PPR protein or fusion protein of the present invention, a functional region may be delivered to the inside of a living body or cells and made to function in a nucleic acid sequence-specific manner. A complex linked with a marker such as GFP may be used to visualize a desired RNA in a living body.

With the PPR protein or fusion protein of the present invention, a nucleic acid can be modified or disrupted in a nucleic acid sequence-specific manner in the inside of cells or living bodies, and a new function may be conferred. In particular, RNA-binding PPR proteins are involved in all the RNA processing steps found in the organelles, such as cleavage, RNA edition, translation, splicing, and RNA stabilization. Accordingly, such uses of the method concerning modification of PPR proteins provided by the present invention, as well as the PPR motif and PPR protein provided by the present invention as mentioned below can be expected in a variety of fields.

### (1) Medical Care

Creation of a PPR protein that recognizes and binds to a specific RNA associated with a specific disease. Analysis of a target sequence and associated proteins for a specific RNA. The results of the analysis can be used to identify compounds for the treatment of the disease.

For example, it is known that, in animals, abnormalities in the PPR protein identified as LRPPRC cause Leigh syndrome, French Canadian type (LSFC, Leigh syndrome, subacute necrotizing encephalomyelopathy). The present invention may contribute to the treatment (prevention, therapeutic treatment, or inhibition of progression) of LSFC. Many of the existing PPR proteins work to specify edition sites for RNA manipulation (conversion of genetic information on RNA, often C to U). The PPR proteins of this type have an additional motif that is suggested to interact with RNA editing enzymes on the C-terminal side. PPR proteins having this structure are expected to enable introduction of base polymorphism or treatment of a disease or condition caused by base polymorphism.
Creation of cells with controlled RNA repression/expression. Such cells include stem cells of which differentiation or undifferentiation state is monitored (e.g., iPS cells), model cells for evaluation of cosmetics, and cells in which the expression of functional RNA can be turned on or off for the purpose of elucidating action mechanism and pharmacological testing for drug discovery.
Preparation of a PPR protein that specifically binds to a specific RNA associated with a particular disease. Such a PPR protein is introduced into a cell using a plasmid, virus vector, mRNA, or purified protein, and an RNA function that causes a disease can be changed (improved) by binding of the PPR protein to the target RNA in the cell. Examples of the mechanism of changing the function include, for example, change of the RNA structure by binding, knockdown by decomposition, change of the splicing reaction by splicing, base substitution, and so forth.

### (2) Agriculture, Forestry and Fishery

Improvement of yield and quality of crops, forest products and marine products.
Breeding of organisms with improved disease resistance, improved environmental tolerance, or improved or new function.

For example, concerning hybrid firstgeneration (F1) plant crops, an F1 plant may be artificially created by using stabilization of mitochondrial RNA and translation control by PPR proteins so that yield and quality of the crops may be improved. RNA manipulation and genome edition using PPR proteins more accurately and quickly enable variety improvement and breeding (genetic improvement of organisms) of organisms compared with conventional techniques. In addition, it can be said that RNA manipulation and genome editing using PPR proteins are similar to the classical breeding methods such as selection of mutants and backcrossing, since they do not transform traits with a foreign gene as in genetic recombination, but they are techniques using RNA and genomes originally possessed by plants and animals. Therefore, they can also surely and quickly cope with global-scale food and environmental problems.

### (3) Chemistry

Control of protein expression amount by manipulating DNA and RNA in the production of useful substances using microorganisms, cultured cells, plant bodies, and animal bodies (e.g., insect bodies). Productivity of useful substances can be thereby improved. Examples of the useful substances are proteinaceous substances such as antibodies, vaccines, and enzymes, as well as relatively low-molecular weight compounds such as pharmaceutical intermediates, fragrances, and dyes.
Improvement of production efficiency of biofuel by modification of metabolic pathways of algae and microorganisms.

### EXAMPLES

### Example 1: Intracellular Analysis of Fluorescent Protein-Fused PPR Proteins

### (Design of Motifs)

The target sequence was CAGCAGCAGCAGCAGCAG (SEQ ID NO: 1) including six repeats of CAG sequence. The base recognized by a PPR motif is determined by the 1st, 4th and ii-th amino acids in the sequence. The PPR motif that recognizes cytosine contained 1st is valine, 4th asparagine, and ii-th serine, the PPR motif that recognizes adenine contained 1st is valine, 4th threonine, and ii-th asparagine, and the PPR motif that recognizes guanine contained 1st is valine, 4th threonine, and ii-th aspartic acid, respectively. For the PPR motif that recognizes uracil, 1st valine, 4th asparagine, and ii-th aspartic acid can be used.

Further, as the 6th and 9th amino acids of the 1st motif that recognizes cytosine (Mutated motif in FIG. 1A), there were selected leucine and glycine (C_6L9G, PPRcag_1, SEQ ID NO: 2 mentioned above) as a typical combination, and leucine and glutamic acid (C_6L9E, PPRcag_2, SEQ ID NO: 3), asparagine and glutamine (C_6N9Q, PPRcag_3, SEQ ID NO: 4), asparagine and glutamic acid (C_6N9E, PPRcag_4, SEQ ID NO: 5), asparagine and lysine (C_6N9K, PPRcag_5, SEQ ID NO: 6), and aspartic acid and glycine (C_6D9G, PPRcag_6, SEQ ID NO: 7) were selected as mutant types (FIG. 1i). These PPR motif sequences were arranged so that the encoded proteins bind to the CAGCAGCAGCAGCAGCAG sequence (SEQ ID NO: 1 mentioned above), and PPR genes (SEQ ID NOS: 11 to 16) were prepared. In order to efficiently and accurately ligate the 18 of DNAs encoding the respective PPR motifs, the highly frequently found amino acids were selected as the amino acids other than the 1st, 4th, 6th, 9th, and ii-th amino acids in the PPR motifs for cytosine, adenine, and guanine, respectively, as described above (SEQ ID NOS: 8 and 9, see Patent document 1 mentioned above).

### (Preparation of Plasmids)

Plasmids containing each of the PPR genes were constructed by using the Golden Gate method. In more detail, 10 kinds of intermediate vectors Dest-a, b, c, d, e, f, g, h, i, and j, designed to be seamlessly ligated in sequence, were prepared, and 20 kinds of motifs consisting of one motif or two motifs (single PPR motifs corresponding to A, C, G, and U, and double PPR motifs corresponding to AA, AC, AG, AU, CA, CC, CG, CU, GA, GC, GG, GU, UA, UC, UG, and UU) were inserted into 10 kinds of the vectors to produce 200 kinds of parts.

There were prepared gaagacataaactccgtggtcacATACagagaccaaggtctcaGTGGtcacatacatgtcttc (SEQ ID NO: 43) as Dest-a, gaagacatATACagagaccaaggtctcaGTGGtgacataatgtcttc (SEQ ID NO: 44) as Dest-b, gaagacatcATACagagaccaaggtctcaGTGGttacatatgtcttc (SEQ ID NO: 45) as Dest-c, gaagacatacATACagagaccaaggtctcaGTGGttacaatgtcttc (SEQ ID NO: 46) as Dest-d, gaagacattacATACagagaccaaggtctcaGTGGtgacatgtcttc (SEQ ID NO: 47) as Dest-e, gaagacattgacATACagagaccaaggtctcaGTGGttaatgtcttc (SEQ ID NO: 48) as Dest-f, gaagacatgttacATACagagaccaaggtctcaGTGGtcatgtcttc (SEQ ID NO: 49) as Dest-g, gaagacatggtcacATACagagaccaaggtctcaGTGGtatgtcttc (SEQ ID NO: 50) as Dest-h, gaagacattggttacATACagagaccaaggtctcaGTGGatgtcttc (SEQ ID NO: 51) as Dest-i, and gaagacatgtggtgacATACagagaccaaggtctcaGTGGtcttc (SEQ ID NO: 52) as Dest-j by a gene synthesis technique, and cloned into pUC57-kan.

Dest-a to Dest-j were selected according to the target sequence, and cloned into the vector by the Golden Gate reaction. The vector used here was designed so that the amino acid sequence of MGNSV (SEQ ID NO: 53) was added to the N-terminus of the 18 PPR sequences linked together, and ELTYNTLISGLGKAGRARDPPV (SEQ ID NO: 54) to the C-terminus of the same. It was confirmed that the correct size genes were cloned, and the sequences of the cloned genes were confirmed by sequencing.

### (Detection of Expression in Cells)

The expression plasmid pcDNA3.1 for expression in cultured animal cells contains the CMV promoter and SV40 poly-A signal sequence, and a gene to be expressed can be inserted between them. To detect the expression of PPR proteins in cells, PPR proteins fused with a fluorescent protein were expressed, and aggregation and nuclear localization thereof in the cells were analyzed on the basis of fluorescent images thereof. Fused protein genes comprising those for EGFP, nuclear localization signal sequence, PPR protein, and FLAG epitope tag fused together in this order from the N-terminus side were inserted into pcDNA3.1 (SEQ ID NOS: 17 to 22). Protein genes comprising those for mClover3, PPR protein, nuclear localization signal sequence, and FLAG epitope tag fused together in this order from the N-terminus side were also inserted into pcDNA3.1 (SEQ ID NOS: 23 to 28). Plasmids not containing PPR were also prepared as control (SEQ ID NOS: 35 and 36).

The HEK293T cells were inoculated at a density of 1×10⁶ cells in 10 cm dish containing 9 mL of DMEM, and 1 mL of FBS, and cultured in an environment of 37° C. and 5% CO₂ for 2 days, and then the cells were collected. The collected cells were inoculated on a PLL-coated 96-well plate at a density of 4×10⁴ cells/well, and cultured in an environment of 37° C. and 5% CO₂ for 1 day. A mixture of 200 ng of the plasmid DNA, 0.6 μL of Fugene (registered trademark)-HD (Promega, E2311), and 200 μL of Opti-MEM was prepared, the whole volume thereof was added to each well, and culture was performed in an environment of 37° C. and 5% CO₂ for 1 day. After the culture, the medium was removed, each well was washed once with 50 μL of PBS, then 1 μL of Hoechst (1 mg/mL, Dojin Chemical, 346-07951) and 50 μL of PBS were added to each well, the plate was left under an environment of 37° C. and 5% CO₂ for 10 minutes, and then each well was washed with 50 μL of PBS. After the washing, 50 μL of PBS was added, and GFP fluorescence and Hoechst fluorescence images of each well were obtained by using a DMi8 fluorescence microscope (Leica).

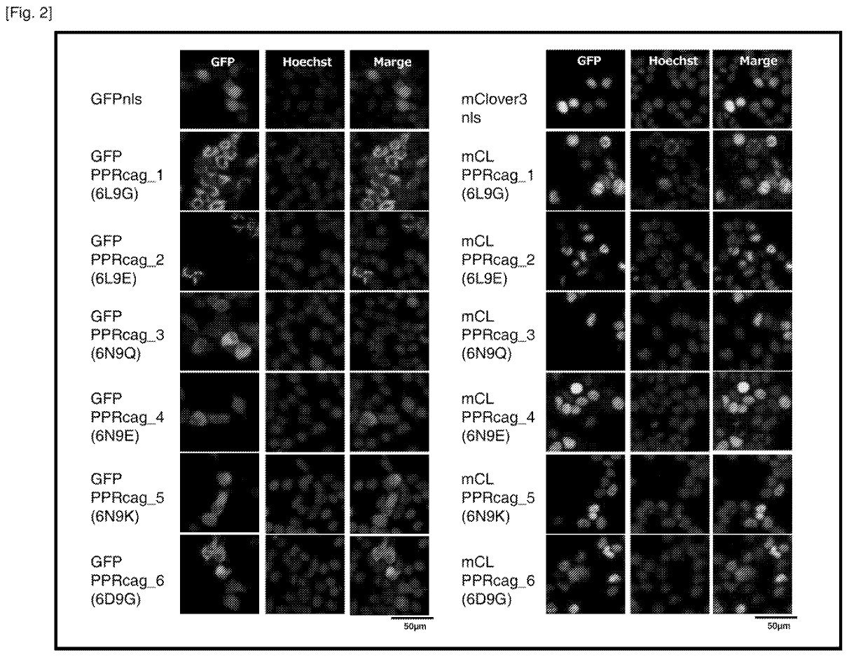

The results are shown in FIG. 2. Expression in the cells of the PPR proteins fused with EGFP and nuclear localization signal sequence was confirmed, and as a result, it was confirmed that PPRcag_1 (6L9G) and PPRcag_2 (6L9E) did not localize to the nuclei, but strongly aggregated around the nuclei. On the other hand, PPRcag_3 (6N9Q), PPRcag_4 (6N9E), PPRcag_5 (6N9K), and PPRcag_6 (6D9G) did not localize to the nuclei, although their aggregation was week. When mClover3 was fused, it was confirmed that PPRcag_1 (6L9G) and PPRcag_2 (6L9E) localized to the nuclei, but aggregated in the nuclei. In contrast, PPRcag_3 (6N9Q), PPRcag_4 (6N9E), PPRcag_5 (6N9K), and PPRcag_6 (6D9G) localized to the nuclei, and did not aggregate. Therefore, it was found that 6N9E, 6N9Q, 6N9K, and 6D9G mutations are favorable for improving aggregation, and mClover3 is better than EGFP for efficient localization to the nuclei.

### Example 2: RNA Binding Analysis of CAG-Binding PPR Proteins

To confirm binding of PPRcag_1, PPRcag_2, PPRcag_3, PPRcag_4, PPRcag_5, and PPRcag_6 to their target RNAs, recombinant proteins were prepared, and binding experiments were performed.

Protein genes were designed for the respective PPR proteins fused with luciferase on the N-terminus side and 6× histidine tag sequence on the C-terminus side, and cloned into an E. coli expression plasmid (SEQ ID NOS: 29 to 34). Nluc-His×6 protein gene not containing PPR protein was also prepared as a control (SEQ ID NOS: 37).

The E. coli Rosetta (DE3) strain was transformed with the completed plasmids. The E. coli was cultured in 2 mL of the LB medium containing 100 μg/mL ampicillin at 37° C. for 12 hours. When OD₆₀₀ reached 0.5 to 0.8, the culture medium was transferred to an incubator at 15° C., and left standing for 30 minutes. Then, 100 μL of an IPTG solution was added (IPTG final concentration, 0.1 mM), and the culture was further continued at 15° C. for 16 hours. An E. coli pellet was collected by centrifugation at 5,000×g and 4° C. for 10 minutes, 1.5 mL of a lysis buffer (20 mM Tris-HCl, pH 8.0, 150 mM NaCl, 0.5% NP-40, 1 mM MgCl₂, 2 mg/mL lysozyme, 1 mM PMSF, 2 μL of DNase) was added to the pellet, and the mixture was frozen at −80° C. for 20 minutes. The cells were cryodisrupted with permeabilization at 25° C. for 30 minutes. The disrupted cell mixture was then centrifuged at 3,700 rpm and 4° C. for 15 minutes, and the supernatant containing soluble PPR protein (E. coli lysate) was collected.

The binding experiment for PPR protein and RNA was performed by an experimental method for binding of PPR protein and biotinylated RNA on a streptavidin plate.

There were synthesized RNA probes of 30-base RNAs containing the target CAG×6 sequence, untargeted CGG×6, CUG×6, and CCG×6, and the D1b (UGGUGUAUCUUGUCUUUA) sequence (positions 8 to 25 of SEQ ID NO: 42) modified with biotin at the 5′-end (in that order, SEQ ID NOS: 38 to 42, respectively) (Grainer). To a streptavidin-coated plate (Thermo Fisher, Cat. No. 15502), the 5′-end biotinylated RNA probes were added, reaction was allowed at room temperature for 30 minutes, and the plate was washed with a probe washing buffer (20 mM Tris-HCl, pH 7.6, 150 mM NaCl, 5 mM MgCl₂, 0.5% NP −40, 1 mM DTT, 0.1% BSA). For background measurement, wells to which biotinylated RNA was not added, but the lysis buffer was added were also prepared. Then, a blocking buffer (20 mM Tris-HCl, pH 7.6, 150 mM NaCl, 5 mM MgCl₂, 0.5% NP-40, 1 mM DTT, 1% BSA) was added, and the plate surface was blocked at room temperature for 30 minutes. Then, 100 μL of the E. coli lysate containing luciferase-fused PPR protein having a luminescence of 1.5×10⁸ LU/μL was added, and the binding reaction was allowed at room temperature for 30 minutes. Then, the plate was washed 5 times with 200 μL of a washing buffer (20 mM Tris-HCl, pH 7.6, 150 mM NaCl, 5 mM MgCl₂, 0.5% NP-40, 1 mM DTT). To each well, 40 μL of luciferase substrate (Promega, E151A) diluted 2,500-fold with the washing buffer was added, reaction was allowed for 5 minutes, and then luminescence was measured with a plate reader (PerkinElmer, Cat. No. 5103-35).

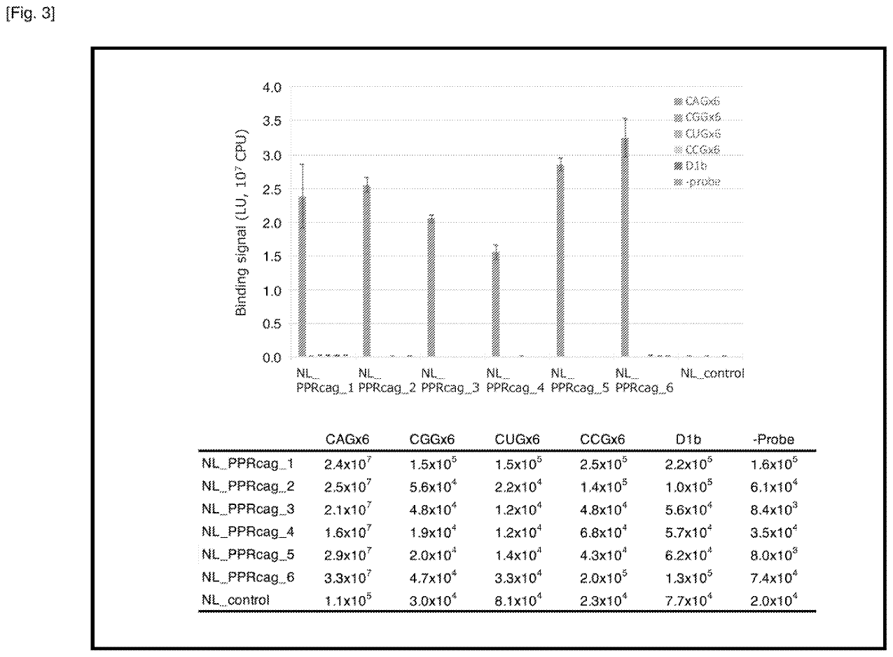

The results are shown in FIG. 3. It was found that all PPRs specifically bind to the target, CAG×6. The binding powers of PPRcag_2, PPRcag_3, PPRcag_4, PPRcag_5, and PPRcag_6 to the target sequence were substantially equivalent, about 80%, about 60%, about 120%, and about 130%, respectively, compared with that of PPRcag_1. These results indicate that the binding performance was not substantially changed by the mutations except for PPRcag_4.

### Example 3: Control of Aggregation of PPR Protein

A PPR protein using V2 motif (SEQ ID NO: 61 for nucleotide sequence, and SEQ ID NO: 62 for amino acid sequence) and a PPR protein using v3.2 motif (SEQ ID NO: 63 for nucleotide sequence, and SEQ ID NO: 64 for amino acid sequence) were prepared in an E. coli expression system, respectively, purified, and separated by gel filtration chromatography. The v2 motif refers to the PPR motifs having the sequence of SEQ ID NO: 2, and SEQ ID NOS: 8 to 10, and the v3.2 motif refers to the PPR motifs having the sequence of SEQ ID NO: 4 and SEQ ID NOS: 58 to 60 in the case of the first motif from the N-terminus side, or in the other cases, the PPR motif for adenine comprising the sequence of SEQ ID NO: 8 having a substitution of 15th aspartic acid with lysine, or the PPR motifs for bases other than adenine comprising a sequence selected from SEQ ID NOS: 2, 9, and 10.

### (Expression and Purification of Proteins)

The E. coli Rosetta strain was transformed with pE-SUMOpro Kan plasmid containing a DNA sequence encoding the objective PPR protein, and cultured at 37° C., then the temperature was lowered to 20° C. when OD₆₀₀ reached 0.6, and IPTG was added at a final concentration of 0.5 mM so that the objective PPR was expressed in the E. coli cells as SUMO-fused protein. The cells were cultured overnight, then collected by centrifugation, and resuspended in a lysis buffer (50 mM Tris-HCl, pH 8.0, 500 mM NaCl). The E. coli cells were disrupted by sonication, and centrifuged at 17,000 g for 30 minutes, then the supernatant fraction was applied to an Ni-Agarose column, the column was washed with the lysis buffer containing 20 mM imidazole, and then the SUMO-fused objective PPR protein was eluted with the lysis buffer containing 400 mM imidazole. After the elution, the SUMO protein was cleaved from the objective PPR protein with Ulp1, and at the same time, the protein solution was substituted with an ion-exchange buffer (50 mM Tris-HCl, pH 8.0, 200 mM NaCl) by dialysis. Subsequently, cation exchange chromatography was performed by using SP column. After application to the column, proteins were eluted with gradually increasing NaCl concentration of from 200 mM to 1 M. The fraction containing the objective PPR protein was subjected to final purification by gel filtration chromatography using Superdex 200 column. The objective PPR protein eluted from the ion exchange column was applied to the gel filtration column equilibrated with a gel filtration buffer (25 mM HEPES, pH 7.5, 200 mM NaCl, 0.5 mM tris(2-carboxyethyl)phosphine (TCEP)). Finally, the fraction containing the objective PPR protein was concentrated, frozen in liquid nitrogen, and stored at −80° C. until used for the next analysis.

### (Gel Filtration Chromatography)

The purified recombinant PPR protein was prepared at a concentration of 1 mg/ml. For gel filtration chromatography, Superdex 200 increase 10/300 GL (GE Healthcare) was used. To the gel filtration column equilibrated with 25 mM HEPES pH7.5, 200 mM NaCl, 0.5 mM tris(2-carboxyethyl)phosphine (TCEP), the prepared protein was applied, and the absorbance of the solution eluted from the gel filtration column was measured at 280 nm to analyze the properties of the protein.

### (Results)

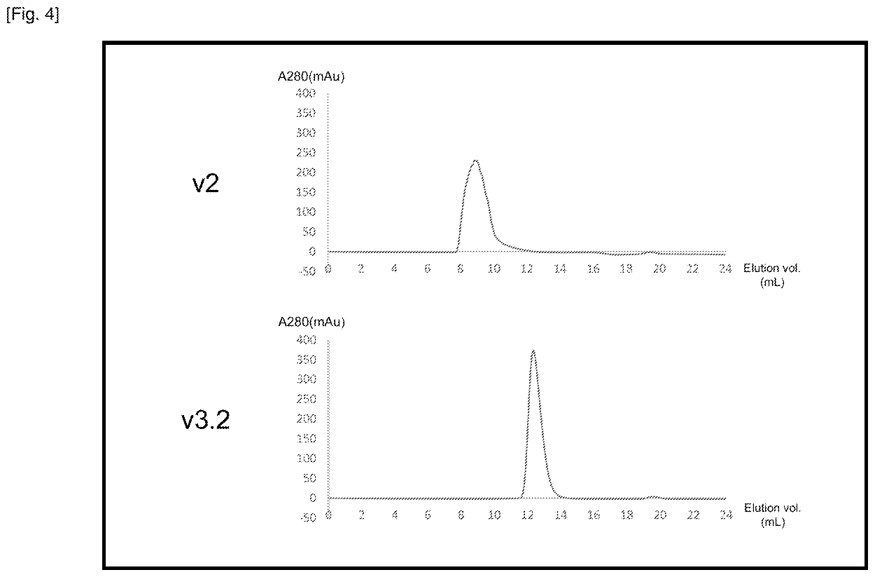

The results are shown in FIG. 4. The smaller volume of the elution fraction (Elution vol.) means a larger molecular size. The proteins using v2 were eluted in 8 to 10 mL of elution fractions, whereas the peaks of the proteins using v3.2 were observed in elution fractions of 12 to 14 mL. This result suggested possibility that the proteins using v2 aggregated due to the larger protein size thereof, and the aggregation was improved in the proteins using v3.2.

1. A nucleic acid encoding the PPR motif according to any one selected from the following group, a PPR protein containing the PPR motif as the first PPR motif from the N-terminus, or the protein:
(C-1) a PPR motif consisting of any one of the sequences of SEQ ID NOS: 4 to 7;
(C-2) a PPR motif consisting of any one of the sequences of SEQ ID NOS: 4 to 7 having a substitution, deletion, or addition of 1 to 9 amino acids other than the amino acids at positions 1, 4, 6, and 34;
(C-3) a PPR motif having a sequence identity of at least 80% to any one of the sequences of SEQ ID NOS: 4 to 7, provided that the amino acids at positions 1, 4, 6, and 34 are identical;
(A-1) a PPR motif consisting of the sequence of SEQ ID NO: 8 having a substitution of the amino acid at position 6 with asparagine or aspartic acid;
(A-2) a PPR motif consisting of the sequence of (A-1) having a substitution, deletion, or addition of 1 to 9 amino acids other than the amino acids at positions 1, 4, 6, and 34;
(A-3) a PPR motif having a sequence identity of at least 80% to the sequence of (A-1), provided that the amino acids at positions 1, 4, 6, and 34 are identical;
(G-1) a PPR motif consisting of the sequence of SEQ ID NO: 9 having a substitution of the amino acid at position 6 with asparagine or aspartic acid;
(G-2) a PPR motif consisting of the sequence of (G-1) having a substitution, deletion, or addition of 1 to 9 amino acids other than the amino acids at positions 1, 4, 6, and 34;
(G-3) a PPR motif having a sequence identity of at least 80% to the sequence of (G-1), provided that the amino acids at positions 1, 4, 6, and 34 are identical;
(U-1) a PPR motif consisting of the sequence of SEQ ID NO: 10 having a substitution of the amino acid at position 6 with asparagine or aspartic acid;
(U-2) a PPR motif consisting of the sequence of (U-1) having a substitution, deletion, or addition of 1 to 9 amino acids other than the amino acids at positions 1, 4, 6, and 34; and
(U-3) a PPR motif having a sequence identity of at least 80% to the sequence of (U-1), provided that the amino acids at positions 1, 4, 6, and 34 are identical;
(A-4) a PPR motif consisting of the sequence of SEQ ID NO: 8 having such substitutions of the amino acids at positions 6 and 9 that any one of the combinations defined below is satisfied;
(G-4) a PPR motif consisting of the sequence of SEQ ID NO: 9 having such substitutions of the amino acids at positions 6 and 9 that any one of the combinations defined below is satisfied;
(U-4) a PPR motif consisting of the sequence of SEQ ID NO: 10 having such substitutions of the amino acids at positions 6 and 9 that any one of the combinations defined below is satisfied;
wherein the conditions for SEQ ID NOs:8-10 of (A-4), (G-4), or (U-4) are selected from
a combination of asparagine as the amino acid at position 6 and glutamic acid as the amino acid at position 9, —a combination of asparagine as the amino acid at position 6 and glutamine as the amino acid at position 9, —a combination of asparagine as the amino acid at position 6 and lysine as the amino acid at position 9, and —a combination of aspartic acid as the amino acid at position 6 and glycine as the amino acid at position 9.

2. A vector comprising the nucleic acid according to claim 1.

3. A cell (except for human individual) containing the vector according to claim 2.

4. A pharmaceutical composition comprising the vector according to claim 2, and an additive.
